(19) United States
(12) Patent Application Publication
(10) Pub. No.: US 2003/0149998 A1
(43) Pub. Date: Aug. 7, 2003

(54) Genes encoding G-protein coupled receptors and methods of use therefor

(75) Inventors: Maria Blatcher, Moorestown, NJ (US); Janet E. Paulsen, Londonderry, NH (US); Brian G. Bates, Chelmsford, MA (US)

Correspondence Address: Bill T. Brazil, Five Giralda Farms, Madison, NJ, 07940-0874, US

(73) Assignee: Wyeth, Madison, NJ

(21) Appl. No.: 10/293,983

(22) Filed: Nov. 13, 2002

Related U.S. Application Data
(60) Provisional application No. 60/332,110, filed on Nov. 16, 2001.

(51) Int. Cl. C12Q 1/68; A01K 67/00; C07H 21/04; C12N 9/64; C12P 21/02; C12N 5/06

(52) U.S. Cl. 800/8; 435/6; 435/69.1; 435/325; 435/320.1; 435/226; 536/23.2

(57) ABSTRACT

The present invention relates generally to the fields of neuroscience, bioinformatics and molecular biology. More particularly, the invention relates to newly identified polynucleotides that encode G-protein coupled receptors (GPCRs), the use of such polynucleotides and polypeptides, as well as the production of such polynucleotides and polypeptides. The invention relates also to identifying compounds which may be agonists, antagonists and/or inhibitors of GPCRs, and therefore potentially useful in therapy.

This application claims priority from copending provisional application serial No. 60/332,110 filed on Nov. 16, 2001.

### FIELD OF THE INVENTION

The present invention relates generally to the fields of neuroscience, bioinformatics and molecular biology. More particularly, the invention relates to newly identified polynucleotides that encode G-protein coupled receptors (GPCRs), the use of such polynucleotides and polypeptides, as well as the production of such polynucleotides and polypeptides. The invention relates also to identifying compounds which may be agonists, antagonists and/or inhibitors of GPCRs, and therefore potentially useful in therapy.

### BACKGROUND OF THE INVENTION

It is well established that many medically significant biological processes are mediated by polypeptides participating in cellular signal transduction pathways that involve G-proteins and second messengers, e.g., cAMP, IP₃ and diacylglycerol (Lefkowitz, 1991). Some examples of these polypeptides include G-proteins themselves (e.g., G-protein families I, II and II), G-protein coupled receptors (GPCRs), such as those for biogenic amine transmitters (e.g., epinephrine, norepinephrine and dopamine) (Kobilka et al., 1987(a); Kobilka et al., 1987(b); Bunzow et al., 1988), effector polypeptides (e.g., phospholipase C, adenyl cyclase and phosphodiesterase) and actuator polypeptides (e.g., polypeptide kinase A and polypeptide kinase C) (Simon et al., 1991).

One particular pathway of cellular signal transduction is the inositol phospholipid pathway. In this pathway, an extracellular signal molecule (e.g., epinephrine) binds to a G-protein coupled receptor (GPCR), which activates the GPCR. The GPCR subsequently associates with a specific trimeric G-protein, wherein the trimer is comprised of α, β and γ polypeptide subunits. In the GPCR/G-protein associated state, there is an exchange of GDP for GTP at the G-protein α-subunit, resulting in the dissociation of the α-subunit from the β/γ subunits. The GTP bound α-subunit is the active state of the polypeptide. The active α-subunit further activates phospholipase C, which catalyzes the cleavage of PIP₂ to IP₃ and diacylglycerol (DAG). The IP₃ and DAG serve as second messengers in further signal amplification (e.g., Ca²⁺ release and phosphorylation). Hydrolysis of GTP to GDP, catalyzed by the G-protein itself, returns the G-protein to its basal, inactive form. Thus, following GPCR binding a signal molecule, the GPCR activates a G-protein. The G-protein serves a dual role, as an intermediate that relays the signal from receptor to effector, and as a clock that controls the duration of the signal.

GPCRs are membrane bound polypeptides, comprising a gene superfamily characterized as having seven putative transmembrane domains. GPCRs can be intracellularly coupled by heterotrimeric G-proteins to various intracellular enzymes, ion channels and transporters (see, Johnson et al., 1989). Different G-protein α-subunits preferentially stimulate particular effectors to modulate various biological functions in a cell.

The G-protein family of coupled receptors include a wide range of biologically active receptors, such as hormone receptors, viral receptors, growth factor receptors and neuroreceptors. Examples of members of this family include, but are not limited to, dopamine, calcitonin, adrenergic, endothelin, cAMP, adenosine, muscarinic acetylcholine, serotonin, histamine, thrombin, kinin, follicle stimulating hormone, opsins, endothelial differentiation gene-1, rhodopsins, odorant, and cytomegalovirus receptors.

The seven transmembrane GPCR domains are believed to represent transmembrane α-helices connected by extracellular or cytoplasmic loops. GPCRs have been characterized as including these seven conserved hydrophobic stretches of about 20 to 30 amino acids, connecting at least eight divergent hydrophilic loops. Most GPCRs (also known as 7TM receptors) have single conserved cysteine residues in each of the first two extracellular loops which form disulfide bonds that are believed to stabilize functional polypeptide structure. The 7 transmembrane regions are designated as TM1, TM2, TM3, TM4, TM5, TM6, and TM7. TM3 has been implicated in several GPCRs as having a ligand binding site, such as the TM3 aspartate residue. TM5 serines, a TM6 asparagine and TM6 or TM7 phenylalanines or tyrosines are also implicated in ligand binding in certain receptor families.

Phosphorylation and lipidation (palmitylation or farnesylation) of cysteine residues can influence signal transduction of some GPCRs. Most GPCRs contain potential phosphorylation sites within the third cytoplasmic loop and/or the carboxy terminus. For several GPCRs, such as the β-adrenoreceptor, phosphorylation by polypeptide kinase A and/or specific receptor kinases mediates receptor desensitization.

Presently, more than 800 GPCRs from various eukaryotic species have been cloned, 140 of which are human GPCRs for which endogenous ligands are known (Stadel et al., 1997). In addition, several hundred therapeutic agents targeting GPCRs such as angiotensin receptors, calcitonin receptors, adrenoceptor receptors, serotonin receptors, leukotriene receptors, oxytocin receptors, prostaglandin receptors, dopamine receptors, histamine receptors, muscarinic acetylcholine receptors, opioid receptors, somatostatin receptors and vasopressin receptors have been successfully introduced onto the market for various indications (see Stadel et al., 1997). This indicates that these receptors have an established, proven history as therapeutic targets. The search for GPCR genes has also identified numerous genes whose products are members of the GPCR family, but for which their natural ligands are not known, commonly refered to as orphan receptors. In fact, more than 100 of the 240 human GPCRs identified (i.e., about 45%) are orphan receptors, and it is estimated that there are at least 400-1000 more GPCR genes that have yet to be identified (Stadel et al., 1997).

Thus, there is clearly a need for the identification and characterization of further orphan GPCRs, their genes and their ligands, which can play a role in preventing, ameliorating or correcting dysfunctions or diseases, including, but not limited to, infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome.

### SUMMARY OF THE INVENTION

The invention relates to newly identified polynucleotides that encode G-protein coupled receptors, herein GPCRs, the use of such polynucleotides and polypeptides, as well as the production of such polynucleotides and polypeptides. The invention relates also to identifying compounds which may be agonists, antagonists and/or inhibitors of GPCRs, and therefore potentially useful in therapy.

In particular embodiments, the invention is directed to an isolated polynucleotide comprising a nucleic acid sequence which encodes a polypeptide comprising the amino acid sequence of SEQ ID NO:4. In another embodiment, the polynucleotide further comprises nucleic acid sequences encoding a heterologous protein.

In another embodiment, the invention is directed to a recombinant expression vector comprising a polynucleotide having a nucleic acid sequence which encodes a polypeptide comprising the amino acid sequence of SEQ ID NO:4. In certain embodiments, the polynucleotide comprises the nucleic acid sequence of SEQ ID NO:1, SEQ ID NO:2 or SEQ ID NO:3. In certain other embodiments, the polynucleotide is selected from the group consisting of DNA, cDNA, genomic DNA, RNA, pre-mRNA and antisense RNA. In other embodiments, the vector DNA is selected from the group consisting of plasmid, episomal, YAC and viral. In yet another embodiment, the polynucleotide is operatively linked to one or more regulatory elements selected from the group consisting of a promoter, an enhancer, a splicing signal, a termination signal, a ribosomal binding signal and a polyadenylation signal.

In one embodiment, the invention is directed to a genetically engineered host cell, transfected, transformed or infected with a recombinant expression vector comprising a polynucleotide having a nucleic acid sequence which encodes a polypeptide comprising the amino acid sequence of SEQ ID NO:4. In a preferred embodiment, the host cell is a mammalian host cell.

In another embodiment, the invention is directed to an isolated polynucleotide comprising a nucleic acid sequence which encodes a polypeptide comprising the amino acid sequence of SEQ ID NO:7. In a particular embodiment, the polynucleotide further comprises nucleic acid sequences encoding a heterologous protein.

In other embodiments, the invention relates to a recombinant expression vector comprising a nucleic acid sequence which encodes a polypeptide comprising the amino acid sequence of SEQ ID NO:7. In particular embodiments, the polynucleotide comprises the nucleic acid sequence of SEQ ID NO:5 or SEQ ID NO:6. In another embodiment, the polynucleotide is selected from the group consisting of DNA, cDNA, genomic DNA, RNA, pre-mRNA and antisense RNA. In still another embodiment, the polynucleotide is operatively linked to one or more regulatory elements selected from the group consisting of a promoter, an enhancer, a splicing signal, a termination signal, a ribosomal binding signal and a polyadenylation signal. In further embodiments, the vector DNA is selected from the group consisting of plasmid, episomal, YAC and viral.

In certain embodiments, the invention is directed to a genetically engineered host cell, transfected, transformed or infected with a recombinant expression vector comprising a nucleic acid sequence which encodes a polypeptide comprising the amino acid sequence of SEQ ID NO:7. In one preferred embodiment, the host cell is a mammalian host cell.

In certain other embodiments, the invention is directed to an isolated polynucleotide comprising a nucleic acid sequence which encodes a polypeptide comprising the amino acid sequence of SEQ ID NO:9. In particular embodiments, the polynucleotide further comprises nucleic acid sequences encoding a heterologous protein.

In another embodiment, the invention is directed to a recombinant expression vector comprising a polynucleotide comprising a nucleic acid sequence which encodes a polypeptide having the amino acid sequence of SEQ ID NO:9. In particular embodiments, the polynucleotide comprises the nucleic acid sequence of SEQ ID NO:8. In further embodiments, the polynucleotide is selected from the group consisting of DNA, cDNA, genomic DNA, RNA, pre-mRNA and antisense RNA. In yet further embodiments, the polynucleotide is operatively linked to one or more regulatory elements selected from the group consisting of a promoter, an enhancer, a splicing signal, a termination signal, a ribosomal binding signal and a polyadenylation signal. In still another embodiment, the vector DNA is selected from the group consisting of plasmid, episomal, YAC and viral.

In one particular embodiment, the invention is directed to a genetically engineered host cell, transfected, transformed or infected with a recombinant expression vector comprising a polynucleotide comprising a nucleic acid sequence which encodes a polypeptide having the amino acid sequence of SEQ ID NO:9. In one preferred embodiment, the host cell is a mammalian host cell.

In still another embodiment, the invention is directed to an isolated polynucleotide comprising a nucleic acid sequence which encodes a polypeptide comprising the amino acid sequence of SEQ ID NO:11. In particular embodiments, the polynucleotide further comprises nucleic acid sequences encoding a heterologous protein.

In another embodiment, the invention is directed to a recombinant expression vector comprising a polynucleotide comprising a nucleic acid sequence which encodes a polypeptide comprising the amino acid sequence of SEQ ID NO:11. In a particular embodiment, the polynucleotide comprises the nucleic acid sequence of SEQ ID NO:10. In a further embodiment, the polynucleotide is selected from the group consisting of DNA, cDNA, genomic DNA, RNA, pre-mRNA and antisense RNA. In still another embodiment, the polynucleotide is operatively linked to one or more regulatory elements selected from the group consisting of a promoter, an enhancer, a splicing signal, a termination signal, a ribosomal binding signal and a polyadenylation signal. In other embodiments, the vector DNA is selected from the group consisting of plasmid, episomal, YAC and viral.

In another embodiment, the invention is directed to a genetically engineered host cell, transfected, transformed or infected with a recombinant expression vector comprising a polynucleotide comprising a nucleic acid sequence which encodes a polypeptide comprising the amino acid sequence of SEQ ID NO:11. In one preferred embodiment, the host cell is a mammalian host cell.

In certain embodiments, the invention provides an isolated polypeptide comprising the amino acid sequence of SEQ ID NO:4, an isolated polypeptide comprising the amino acid sequence of SEQ ID NO:7, an isolated polypeptide comprising the amino acid sequence of SEQ ID NO:9, and an isolated polypeptide comprising the amino acid sequence of SEQ ID NO:11.

In certain other embodiments, the invention provides an isolated polynucleotide comprising the nucleic acid sequence of SEQ ID NO:1 or a degenerate variant thereof. In particular embodiments, the polynucleotide coding region of SEQ ID NO:1 comprises nucleotides 298 through 1,653.

In one preferred embodiment, the invention is directed to an RNA molecule which is antisense to a polynucleotide comprising the nucleic acid sequence of SEQ ID NO:1 or a degenerate variant thereof. In a particular embodiment, the RNA is antisense to the polynucleotide of SEQ ID NO:1 from about nucleotide 1 to about nucleotide 297 or from about nucleotide 1,654 to about nucleotide 3,824.

In another preferred embodiment, the invention is directed to an isolated polynucleotide comprising the nucleic acid sequence of SEQ ID NO:2 or a degenerate variant thereof. In a particular embodiment, the polynucleotide coding region of SEQ ID NO:2 comprises nucleotides 1 through 1,313.

In another preferred embodiment, the invention is directed to an RNA molecule which is antisense to a polynucleotide comprising the nucleic acid sequence of SEQ ID NO:2 or a degenerate variant thereof. In a particular embodiment, the RNA is antisense to the polynucleotide of SEQ ID NO:2 from about nucleotide 1,314 to about nucleotide 3,405.

In another preferred embodiment, the invention is directed an isolated polynucleotide comprising the nucleic acid sequence of SEQ ID NO:3 or a degenerate variant thereof. In a particular embodiment, the polynucleotide coding region of SEQ ID NO:3 comprises nucleotides 671 through 2,026.

In another preferred embodiment, the invention is directed to an RNA molecule which is antisense to a polynucleotide comprising the nucleic acid sequence of SEQ ID NO:3 or a degenerate variant thereof. In a particular embodiment, the RNA is antisense to the polynucleotide of SEQ ID NO:3 from about nucleotide 1 to about nucleotide 670 or from about nucleotide 2,027 to about nucleotide 3,779.

In still another preferred embodiment, the invention is directed to an isolated polynucleotide comprising the nucleic acid sequence of SEQ ID NO:5 or a degenerate variant thereof. In a particular embodiment, the polynucleotide coding region of SEQ ID NO:5 comprises nucleotides 684 through 2,033.

In another preferred embodiment, the invention is directed to an RNA molecule which is antisense to a polynucleotide comprising the nucleic acid sequence of SEQ ID NO:5 or a degenerate variant thereof. In particular embodiments, the RNA is antisense to the polynucleotide of SEQ ID NO:5 from about nucleotide 1 to about nucleotide 683 or from about nucleotide 2,034 to about nucleotide 3,384.

In yet another preferred embodiment, the invention is directed to an isolated polynucleotide comprising the nucleic acid sequence of SEQ ID NO:6 or a degenerate variant thereof. In particular embodiments, the polynucleotide coding region of SEQ ID NO:6 comprises nucleotides 685 through 2,034.

In other preferred embodiments, the invention is directed to an RNA molecule which is antisense to a polynucleotide comprising the nucleic acid sequence of SEQ ID NO:6 or a degenerate variant thereof. In particular embodiments, the RNA is antisense to the polynucleotide of SEQ ID NO:6 from about nucleotide 1 to about nucleotide 684 or from about nucleotide 2,034 to about nucleotide 3,384.

In yet another preferred embodiment, the invention is directed to an isolated polynucleotide comprising the nucleic acid sequence of SEQ ID NO:8 or a degenerate variant thereof. In a particular embodiment, the polynucleotide coding region of SEQ ID NO:8 comprises nucleotides 332 through 1,858.

In certain preferred embodiments, the invention is directed to an RNA molecule which is antisense to a polynucleotide comprising the nucleic acid sequence of SEQ ID NO:8 or a degenerate variant thereof. In a particular embodiment, the RNA is antisense to the polynucleotide of SEQ ID NO:8 from about nucleotide 1 to about nucleotide 331 or from about nucleotide 1,859 to about nucleotide 4,718.

In further preferred embodiments, the invention is directed to an isolated polynucleotide comprising the nucleic acid sequence of SEQ ID NO:10 or a degenerate variant thereof. In a particular embodiment, the polynucleotide coding region of SEQ ID NO:10 comprises nucleotides 250 through 1,785.

In yet other preferred embodiments, the invention is directed to an RNA molecule which is antisense to a polynucleotide comprising the nucleic acid sequence of SEQ ID NO:10 or a degenerate variant thereof. In certain embodiments, the RNA is antisense to the polynucleotide of SEQ ID NO:10 from about nucleotide 1 to about nucleotide 249 or from about nucleotide 1,786 to about nucleotide 5,386.

In particularly preferred embodiments, the invention is directed to a polynucleotide comprising a nucleic acid sequence which would hybridize to SEQ ID NO:1, or the complement of SEQ ID NO:1, under stringent conditions, a polynucleotide comprising a nucleic acid sequence which would hybridize to SEQ ID NO:2, or the complement of SEQ ID NO:2, under stringent conditions, a polynucleotide comprising a nucleic acid sequence which would hybridize to SEQ ID NO:3, or the complement of SEQ ID NO:3, under stringent conditions, a polynucleotide comprising a nucleic acid sequence which would hybridize to SEQ ID NO:5, or the complement of SEQ ID NO:5, under stringent conditions, a polynucleotide comprising a nucleic acid sequence which would hybridize to SEQ ID NO:6, or the complement of SEQ ID NO:6, under stringent conditions, a polynucleotide comprising a nucleic acid sequence which would hybridize to SEQ ID NO:8, or the complement of SEQ ID NO:8, under stringent conditions, or a polynucleotide comprising a nucleic acid sequence which would hybridize to SEQ ID NO:10, or the complement of SEQ. ID NO:10, under stringent conditions.

In other embodiments, the invention is directed to an antibody which selectively binds to a protein having an amino acid sequence of SEQ ID NO:4, SEQ ID NO:7, SEQ ID NO:9 or SEQ ID NO:11.

In yet other embodiments, the invention is related to transgenic animals comprising a polynucleotide encoding a GPCR polypeptide comprising the amino acid sequence selected from the group consisting of SEQ ID NO:4, SEQ ID NO:7, SEQ ID NO:9 and SEQ ID NO:11. In particular embodiments, the animal is selected from the group consisting of mouse, rat, rabbit and hamster. In other embodiments, the polynucleotide is under the control of a regulatable expression system. In a preferred embodiment, the polynucleotide comprises a mutation which modulates GPCR activity. In another preferred embodiment, the animal is heterozygous for the mutation. In still another preferred embodiment, the animal is homozygous for the mutation.

In other embodiments, the invention provides a method for inhibiting the expression of a GPCR polynucleotide in a cell, the polynucleotide selected from the group consisting of SEQ ID NO:1, SEQ ID NO:2, SEQ ID NO:3, SEQ ID NO:5, SEQ ID NO:6, SEQ ID NO:8 and SEQ ID NO:10, the method comprising provided the cell with a nucleic acid molecule antisense to the polynucleotide.

In another embodiment, the invention is directed to a method for assaying the effects of test compounds on the activity of a GPCR polypeptide comprising the steps of providing a transgenic animal comprising a polynucleotide encoding a GPCR polypeptide having an amino acid sequence selected from the group consisting of SEQ ID NO:4, SEQ ID NO:7, SEQ ID NO:9 and SEQ ID NO:11, administering a test compound to the animal and determining the effects of the test compound on the activity of the GPCR in the presence and absence of the test compound. In particular embodiments, the polynucleotide has at least one mutation selected from the group consisting of nucleotide deletion, nucleotide substitution and nucleotide insertion.

In another embodiment, the invention provides a method for assaying the effects of test compounds on the activity of a GPCR polypeptide comprising the steps of providing recombinant cells comprising a GPCR polypeptide having an amino acid sequence selected from the group consisting of SEQ ID NO:4, SEQ ID NO:7, SEQ ID NO:9 and SEQ ID NO:11, contacting the cells with a test compound and determining the effects of the test compound on the activity of the GPCR in the presence and absence of the test compound. In a preferred embodiment, the determining the effects of the test compound are selected from the group consisting of measuring GPCR kinase activity, measuring GPCR phosphorylation, measuring phosphatidyl inositol levels, measuring GTPase activity, measuring GTP levels, measuring cAMP levels, measuring GDP levels and measuring Ca²⁺ levels. In another embodiment, the polynucleotide has at least one mutation selected from the group consisting of nucleotide deletion, nucleotide substitution and nucleotide insertion.

In further embodiments, the invention is directed to a method for the treatment of a subject in need of enhanced GPCR activity comprising administering to the subject a therapeutically effective amount of an agonist to the GPCR receptor and/or administering to the subject a polynucleotide encoding a GPCR polypeptide comprising an amino acid sequence selected from the group consisting of SEQ ID NO:4, SEQ ID NO:7, SEQ ID NO:9 and SEQ ID NO:11, in a form so as to effect the production of the GPCR activity in vivo.

In another embodiment, the invention is directed to a method for the treatment of a subject in need of inhibiting GPCR activity comprising administering to the subject a therapeutically effective amount of an antagonist to the GPCR receptor and/or administering to the subject a polynucleotide that inhibits the expression of a polynucleotide encoding a GPCR polypeptide comprising an amino acid sequence selected from the group consisting of SEQ ID NO:4, SEQ ID NO:7, SEQ ID NO:9 and SEQ ID NO:11 and/or administering to the subject a therapeutically effective amount of a polypeptide that competes with a GPCR for its ligand.

In yet another embodiment, the invention provides a method for the diagnosis of a disease or the susceptibility to a disease in a subject related to the expression or activity of a GPCR in the subject comprising determining the presence or absence of a mutation in a polynucleotide encoding a GPCR polypeptide comprising an amino acid sequence selected from the group consisting of SEQ ID NO:4, SEQ ID NO:7, SEQ ID NO:9, and SEQ ID NO:11 and/or assaying for the presence of GPCR expression in a sampled derived from the subject, wherein the GPCR expressed is a polynucleotide encoding a GPCR polypeptide comprising an amino acid sequence selected from the group consisting of SEQ ID NO:4, SEQ ID NO:7, SEQ ID NO:9, and SEQ ID NO:11.

In yet another embodiment, the invention provides a method for the treatment of a subject having in need of the inhibition of GPCR activity, such treatment comprising administering to the patient a therapeutically effective amount of an antibody which binds to an extracellular portion of a GPCR polypeptide comprising an amino acid sequence selected from the group consisting of SEQ ID NO:4, SEQ ID NO:7, SEQ ID NO:9, and SEQ ID NO:11.

Other features and advantages of the invention will be apparent from the following detailed description, from the preferred embodiments thereof, and from the claims.

### BRIEF DESCRIPTION OF THE DRAWINGS

FIG. 1 shows an amino acid sequence alignment of human and mouse UP_{—}11 predicted protein sequences.

FIG. 2 shows hydropathy profiles for UP_{—}11 and OM_{—}10. The plots were generated using Toppred and the GES scoring system (Engelman et al., 1986). A significance cut-off score of 1.0 is shown as a solid line.

FIG. 3 shows UP_{—}11 GPCR from genomic prediction and expression patterns.

FIG. 4 shows OM_{—}10 GPCR from genomic prediction and expression patterns.

FIG. 5 shows a human OM_{—}10 cDNA and gene map.

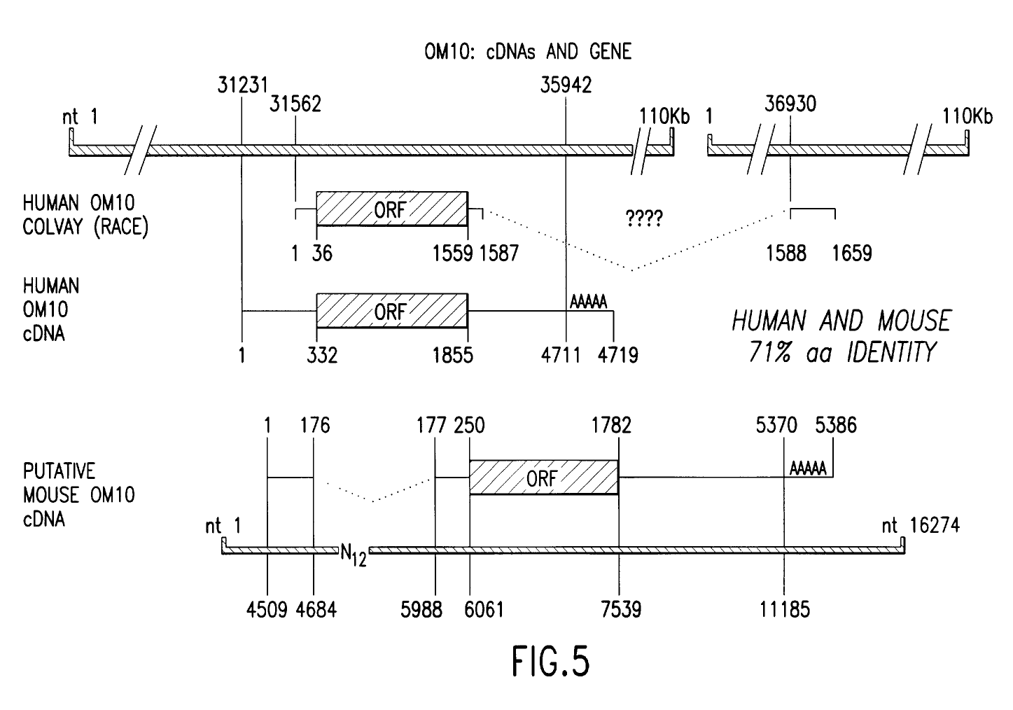

### DETAILED DESCRIPTION OF THE INVENTION

The present invention identifies genes encoding two novel G-protein coupled receptors, hereinafter GPCRs. More particularly, in certain embodiments, the invention is directed to newly identified human genomic polynucleotides, which encode orphan GPCRs designated UP_{—}11 and OM_{—}10. In other embodiments, the invention is directed to the murine orthologs of the above identified human polynucleotides, which encode orphan GPCRs designated mUP_{—}11 and mOM_{—}10. An orphan receptor as defined herein, is a GPCR polypeptide whose naturally occurring ligands have not been identified.

The orphan GPCRs of the invention were identified by a TBLASTN (Altschul et al., 1997) search against the High Throughput Genomic Sequences (HTGS) section of Genbank, and against the Celera Human Genome Database. The search was performed using the human 5-HT₆ receptor sequence (Accession Number L41147). The results of the above TBLASTN search were parsed using a perl script to identify high scoring segment pair protein (HSP) sequences, and these were then searched against a comprehensive database of protein sequences using the BLASTP algorithm. The hits from this secondary BLAST search were then ordered according to E (Expect) value, and each hit was assessed manually for potential novelty based on the degree of similarity to the top database hit. This lead to the identification of several regions of human genomic DNA potentially containing novel GPCRs. These regions of genomic DNA were extracted from the database, and the algorithm Genscan (Burge and Karlin, 1997) was used to predict full-length genes for each of the potential novels. These full length gene predictions were used to design primers and probes for the isolation of full-length cDNA sequences.

The human GPCR polypeptide sequence designated OM_{—}10 (SEQ ID NO:9) is predicted to be encoded by a single exon, starting at nucleotide 332 and ending at nucleotide 1,858 of SEQ ID NO:8. The closest database homologue of OM_{—}10 is “RE2”, also known as the “human H2 histamine receptor” (International Application Nos. WO 00/06597; WO 00/040724; WO 98/20040), which contains two amino acid stretches of similarity that are 32% identical over 192 amino acids within amino acid 31 to amino acid 220 of SEQ ID NO:9 and 32% identical over 76 amino acids within amino acid 394 to amino acid 468 of SEQ ID NO:9. The intervening amino acid stretch of amino acid residue 220 to amino acid residue 394 of SEQ ID NO:9 shares homology with IGS1 (International Application No. WO 01/09184). This region is predicted to encode the third intracellular loop of the OM_{—}10 polypeptide, which is the most variable region in members of the GPCR superfamily. The murine GPCR orthologue (SEQ ID NO:10) of the human OM_{—}10 polypeptide encodes the mOM_{—}10 polypeptide shown in SEQ ID NO:11. The coding sequence of the mOM_{—}10 polynucleotide comprises nucleotides 250 to 1,785 of SEQ ID NO:10.

The human GPCR polypeptide sequence designated UP_{—}11 (SEQ ID NO:4) comprises 451 amino acid residues and is predicted to be encoded by a total of 3 exons. The human UP_{—}11 cDNA polynucleotide sequence of SEQ ID NO:1 (also designated as clone 179), has 82% sequence identity to the human receptor GPR61 (Lee et al., 2000), with a coding sequence from nucleotides 298 to 1,653 of SEQ ID NO:1, an intron at nucleotide position 1,920 and a deletion at nucleotide position 2,879. The amino acid sequence encoded by exon 2 of UP_{—}11 is identical to 232 amino acid residues of the rabbit “G-protein conjugate receptor protein”, described in Japanese Application No. JP08245697 and International Application No. WO 96/05302. The human UP_{—}11 partial cDNA sequence of SEQ ID NO:2 (also designated as clone 200) has a coding sequence from nucleotides 1 to 1,313 and an intron at nucleotide position 1,593. The human UP_{—}11 cDNA sequence of SEQ ID NO:3 (also designated as clone 30) has a coding sequence from nucleotides 671 to 2,026 and an intron at nucleotide position 70 and 2,288.

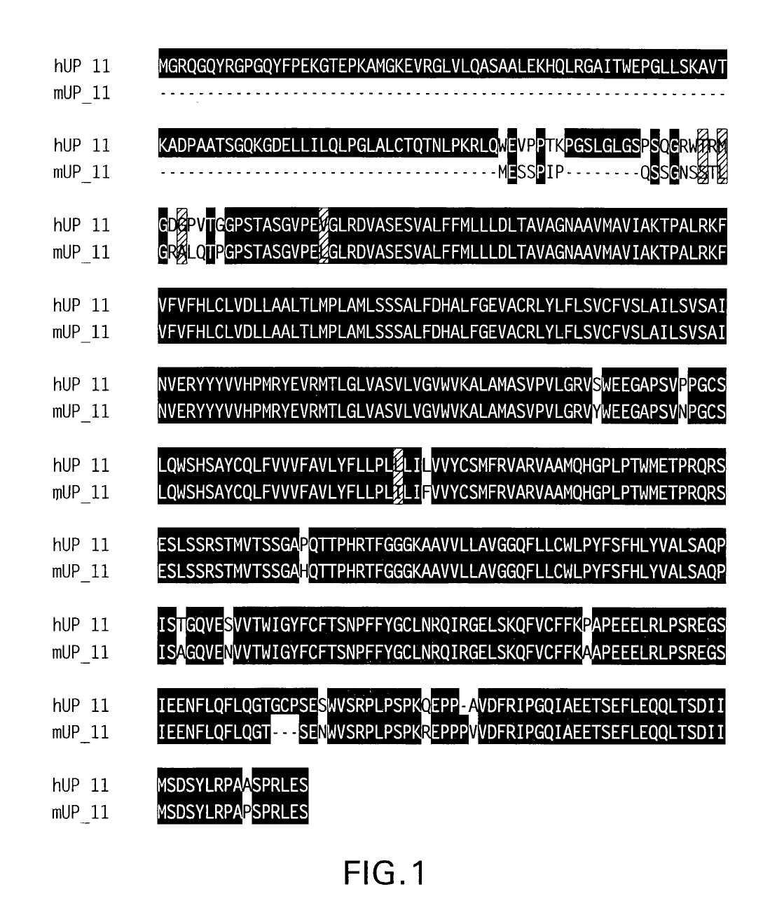

In addition, a 3,384 nucleotide cDNA sequence of SEQ ID NO:5 (clone 67.1) and a 3,397 nucleotide cDNA of SEQ ID NO:6 (clone 52.1) containing the mouse mUP-11 sequence were isolated. The nucleotide coding sequence of SEQ ID NO:5 comprises nucleotides 684 to 2,033 and the nucleotide coding sequence of SEQ ID NO:6 comprises nucleotides 685 to 2,034. Analysis of the mUP_{—}11 sequence demonstrated that mUP-11 contains a single coding exon with high amino acid sequence similarity (i.e., 94% identity) to human UP_{—}11 (FIG. 1).

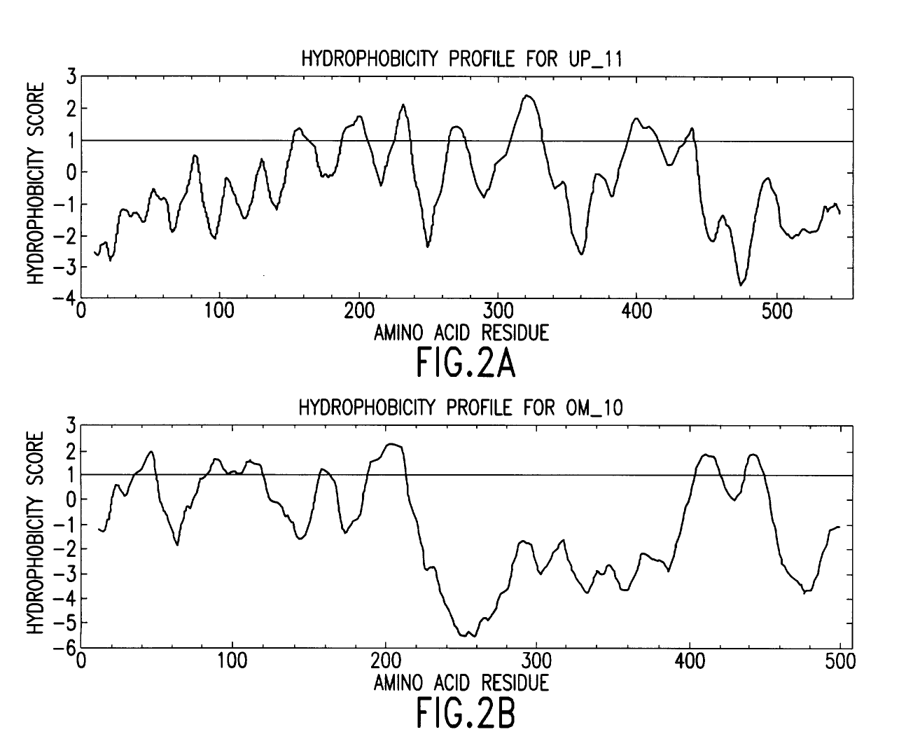

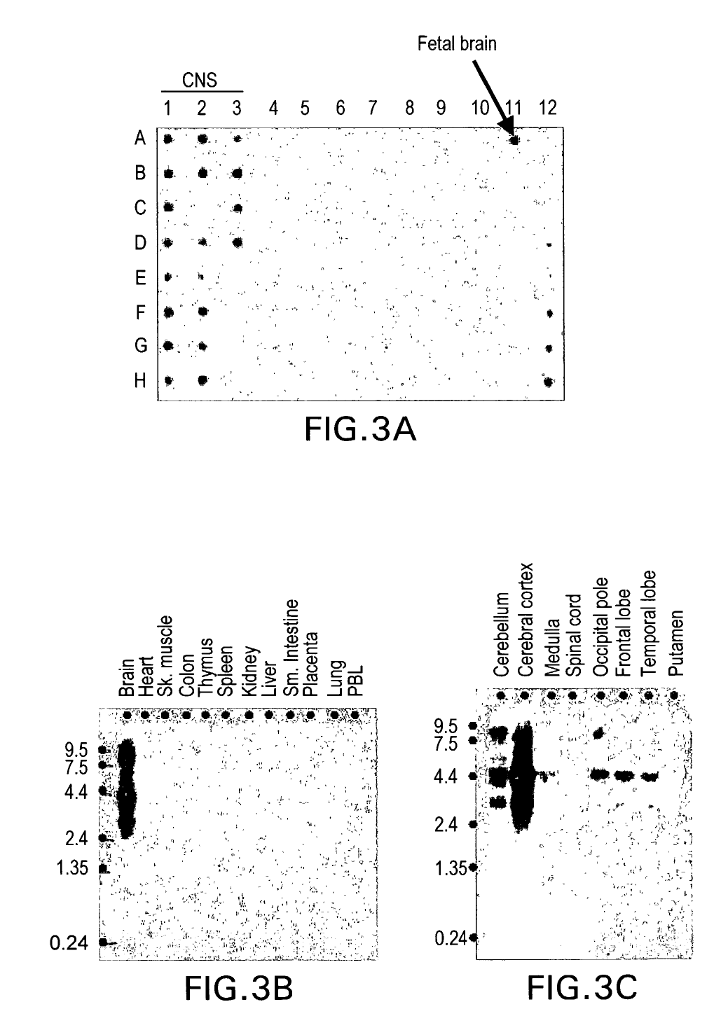

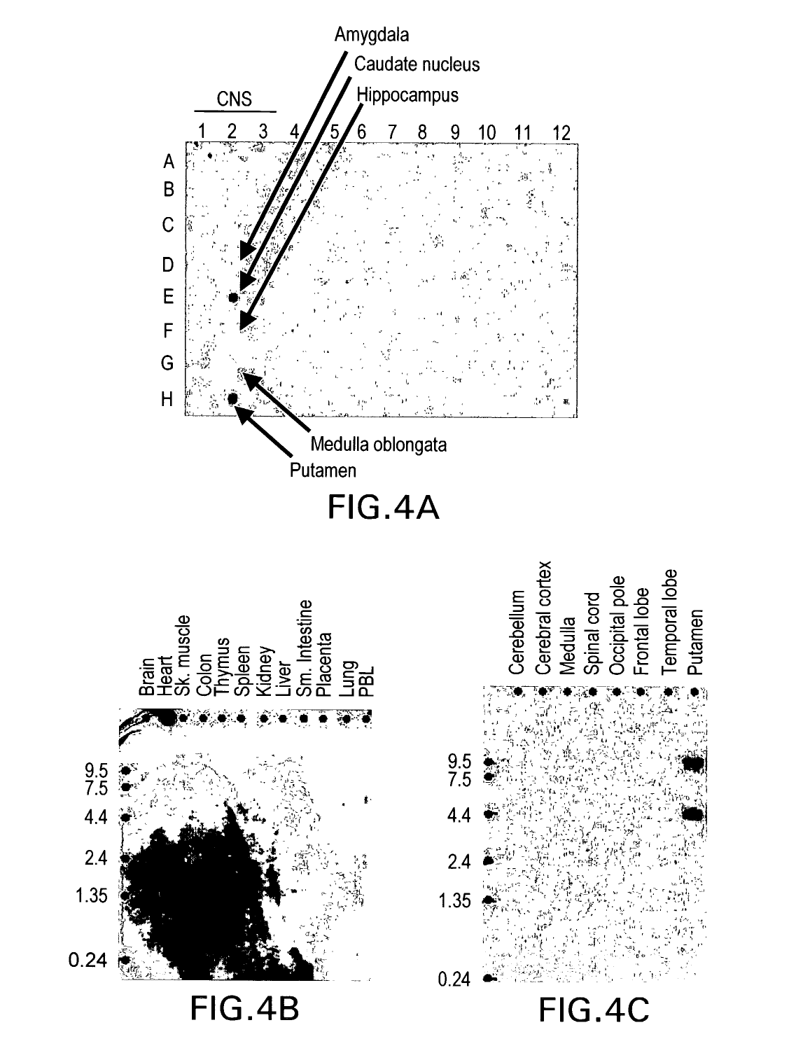

Hydropathy plots of UP_{—}11 and OM_{—}10 (FIG. 2) suggest the presence of 7 transmembrane (TM) domains. In addition to the 7 TM domains, the UP_{—}11 and OM_{—}10 polypeptides contain a number of characteristic motifs which further suggest they belong to the GPCR super-family. For example, both UP_{—}11 and OM_{—}10 contain a conserved aspartate in transmembrane region 2, conserved cysteine residues in the first 2 extracellular loops, a conserved DRY triplet adjacent to transmembrane region 3 (D is conservatively substituted by E in UP_{—}11), as well as numerous other residues known to be important for GPCR structure and function. Expression analysis of UP_{—}11 (FIG. 3) and OM_{—}10 (FIG. 4) indicates that both genes are expressed at high levels in the central nervous system. UP_{—}11 expression was observed in cerebral cortex, frontal lobe, parietal lobe, occipital lobe, temporal lobe, paracentral gyrus of cerebral cortex, pons, cerebellum, corpus callosum, amygdala, caudate nucleus, hippocampus, medulla oblongata, putamen, substantia nigra, accumbens nucleus, thalamus, pituitary gland and the spinal cord when assayed by a tissue expression array. UP_{—}11 transcripts were predominately detected in the brain, as well as detectable in skeletal muscle and heart by multiple tissue Northern analysis. Three UP_{—}11 transcripts were detected on the Northern blots, indicating that the transcripts may be derived from alternate use of exons. mUP_{—}11 transcript was detected in mouse whole brain, olfactory bulb, striatum, cortex, hippocampus, colliculus, midbrain and cerebellum. OM_{—}10 was found to be predominately expressed in the putamen and caudate nucleus. Weaker expression was also seen in amygdala, hippocampus and medulla. Two OM_{—}10 transcripts were detected in the putatem. The mOM_{—}10 transcript was detected in the striatum, midbrain, hypothalamus, brain stem and colliculus.

Thus, in certain embodiments the present invention relates to isolated polynucleotides comprising a nucleic acid sequence of SEQ ID NO:1, SEQ ID NO:2, SEQ ID NO:3, SEQ ID NO:5, SEQ ID NO:6, SEQ ID NO:8 or SEQ ID NO:10, encoding GPCR polypeptides or fragments thereof. In other embodiments the invention relates to GPCR polypeptides comprising an amino acid sequence of SEQ ID NO:4, SEQ ID NO:7, SEQ ID NO:9 or SEQ ID NO:11. In other embodiments the invention relates to polynucleotides encoding GPCR polypeptides comprising an amino acid sequence of SEQ ID NO:4, SEQ ID NO:7, SEQ ID NO:9 or SEQ ID NO:11. In yet other embodiments, the invention provides recombinant vectors comprising a polynucleotide encoding a GPCR polypeptide. In another embodiment, a vector comprising a polynucleotide encoding a GPCR polypeptide is comprised within a host cell, wherein the vector expresses the polynucleotide to produce the encoded polypeptide or fragment thereof. In further embodiments, methods for assaying test compounds for their ability to modulate the activity of GPCR polypeptides, methods for producing GPCR polypeptides, and methods for the diagnosis of a disease or the susceptibility to a disease in a subject related to the expression or activity of a GPCR are provided, as well as methods for treating a subject in need of inhibiting or activating GPCR activity.

A. Isolated Polynucleotides Encoding UP_{—}11 and OM_{—}10 GPCR Polypeptides

Isolated and purified GPCR polynucleotides of the present invention are contemplated for use in the production of GPCR polypeptides. Thus, in one aspect, the present invention provides isolated and purified polynucleotides that encode UP_{—}11 or OM_{—}10 polypeptides. An UP_{—}11 polypeptide is defined as a polypeptide comprising the amino acid sequence depicted in SEQ ID NO:4 (human UP_{—}11), allelic variants of human UP_{—}11, and orthologues of the human UP_{—}11 polypeptide such as the amino acid sequence depicted in SEQ ID NO:7 (mUP_{—}11). An OM_{—}10 polypeptide is defined as a polypeptide comprising the amino acid sequence depicted in SEQ ID NO:9 (human OM_{—}10), allelic variants of human OM_{—}10, and orthologues of the human OM_{—}10 polypeptide such as the amino acid sequence depicted in SEQ ID NO:11 (mOM_{—}10).

Thus, in particular embodiments, a polynucleotide of the present invention is a DNA molecule. In a preferred embodiment, a polynucleotide of the present invention encodes an UP_{—}11 polypeptide comprising the amino acid sequence of SEQ ID NO:4 or SEQ ID NO:7, a variant thereof, or a fragment thereof. In another preferred embodiment, a polynucleotide of the present invention encodes an OM_{—}10 polypeptide comprising the amino acid sequence of SEQ ID NO:9 or SEQ ID NO:11, a variant thereof, or a fragment thereof.

In another aspect of the invention, an isolated and purified polynucleotide comprises a nucleic acid sequence selected from the group consisting of SEQ ID NO:1, SEQ ID NO:2, SEQ ID NO:3, SEQ ID NO:5, SEQ ID NO:6, SEQ ID NO:8 and SEQ ID NO:10, a degenerate variant thereof, or a complement thereof.

A preferred UP_{—}11 polynucleotide comprises the nucleotide sequence shown in SEQ ID NO:1, SEQ ID NO:2, SEQ ID NO:3, SEQ ID NO:5 and SEQ ID NO:6. The sequences of SEQ ID NO:1, SEQ ID NO:2 and SEQ ID NO:3 correspond to human UP_{—}11 cDNAs. These cDNAs comprise sequences encoding the human UP_{—}11 polypeptide (e.g., “the coding region,” from nucleotides 298 to 2,879 of SEQ ID NO:1), as well as 5′ untranslated sequences (nucleotides 1 to 297 of SEQ ID NO:1) and 3′ untranslated sequences (nucleotides 1654 to 3,824 of SEQ ID NO:1). The sequences of SEQ ID NO:5 and SEQ ID NO:6 correspond to cDNAs encoding the murine orthologue of human UP_{—}11.

A preferred OM_{—}10 polynucleotide comprises the nucleotide sequence shown in SEQ ID NO:8 and SEQ ID NO:10. The sequence of SEQ ID NO:8 corresponds to the human OM_{—}10 cDNA. This cDNA comprises sequences encoding the human OM_{—}10 polypeptide (e.g., “the coding region,” from nucleotides 332 to 1858 of SEQ ID NO:8), as well as 5′ untranslated sequences (nucleotides 1 to 331 of SEQ ID NO:8) and 3′ untranslated sequences (nucleotides 1,859 to 4,718 of SEQ ID NO:8). The sequence of SEQ ID NO:10 corresponds to a cDNA encoding the murine orthologue of the human OM_{—}10.

Alternatively, the polynucleotides of the invention can comprise only the coding region of SEQ ID NO:1, SEQ ID NO:2, SEQ ID NO:3, SEQ ID NO:5, SEQ ID NO:6, SEQ ID NO:8 or SEQ ID NO:10.

As used herein, the term “polynucleotide” means a sequence of nucleotides connected by phosphodiester linkages. Polynucleotides are presented herein in the direction from the 5′ to the 3′ direction. A polynucleotide of the present invention can comprise from about 40 to about several hundred thousand base pairs. Preferably, a polynucleotide comprises from about 10 to about 3,000 base pairs. Preferred lengths of particular polynucleotide are set forth hereinafter.

A polynucleotide of the present invention can be a deoxyribonucleic acid (DNA) molecule, a ribonucleic acid (RNA) molecule, or analogs of the DNA or RNA generated using nucleotide analogs. The nucleic acid molecule can be single-stranded or double-stranded, but preferably is double-stranded DNA. Where a polynucleotide is a DNA molecule, that molecule can be a gene, a cDNA molecule or a genomic DNA molecule. Nucleotide bases are indicated herein by a single letter code: adenine (A), guanine (G), thymine (T), cytosine (C), inosine (I) and uracil (U).

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

Preferably, an “isolated” polynucleotide is free of sequences which naturally flank the nucleic acid (i.e., sequences located at the 5′ and 3′ ends of the nucleic acid) in the genomic DNA of the organism from which the nucleic acid is derived. For example, in various embodiments, the isolated GPCR nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., neuronal or placenta). However, the GPCR nucleic acid molecule can be fused to other protein encoding or regulatory sequences and still be considered isolated.

Polynucleotides of the present invention may be obtained, using standard cloning and screening techniques, from a cDNA library derived from mRNA from human cells or from genomic DNA. Polynucleotides of the invention can also synthesized using well known and commercially available techniques.

The invention further encompasses nucleic acid molecules that differ from the nucleotide sequence shown in SEQ ID NO:1, SEQ ID NO:2, SEQ ID NO:3, SEQ ID NO:5, SEQ ID NO:6, SEQ ID NO:8 or SEQ ID NO:10 (and fragments thereof) due to degeneracy of the genetic code and thus encode the same GPCR polypeptide as that encoded by the nucleotide sequence shown in SEQ ID NO:1, SEQ ID NO:2, SEQ ID NO:3, SEQ ID NO:5, SEQ ID NO:6, SEQ ID NO:8 or SEQ ID NO:10.

In another preferred embodiment, an isolated polynucleotide of the invention comprises a nucleic acid molecule which is a complement of the nucleotide sequence shown in SEQ ID NO:1, SEQ ID NO:2, SEQ ID NO:3, SEQ ID NO:5, SEQ ID NO:6, SEQ ID NO:8 or SEQ ID NO:10, or a fragment of these nucleotide sequences. A nucleic acid molecule which is complementary to the nucleotide sequence shown in SEQ ID NO:1, SEQ ID NO:2, SEQ ID NO:3, SEQ ID NO:5, SEQ ID NO:6, SEQ ID NO:8 or SEQ ID NO:10 is one which is sufficiently complementary to the nucleotide sequence of SEQ ID NO:1, SEQ ID NO:2, SEQ ID NO:3, SEQ ID NO:5, SEQ ID NO:6, SEQ ID NO:8 or SEQ ID NO:10, such that it can hybridize to the nucleotide sequence shown in SEQ ID NO:1, SEQ ID NO:2, SEQ ID NO:3, SEQ ID NO:5, SEQ ID NO:6, SEQ ID NO:8 or SEQ ID NO:10, thereby forming a stable duplex.

Orthologues and allelic variants of the human and murine UP_{—}11 and OM_{—}10 polynucleotides can readily be identified using methods well known in the art. Allelic variants and orthologues of these GPCRs will comprise a nucleotide sequence that is typically at least about 70-75%, more typically at least about 80-85%, and most typically at least about 90-95% or more homologous to the nucleotide sequence shown in SEQ ID NO:1, SEQ ID NO:2, SEQ ID NO:3, SEQ ID NO:5, SEQ ID NO:6, SEQ ID NO:8 or SEQ ID NO:10, or a fragment of these nucleotide sequences. Such nucleic acid molecules can readily be identified as being able to hybridize, preferably under stringent conditions, to the nucleotide sequence shown in SEQ ID NO:1, SEQ ID NO:2, SEQ ID NO:3, SEQ ID NO:5, SEQ ID NO:6, SEQ ID NO:8 or SEQ ID NO:10, or a fragment of these nucleotide sequences.

Moreover, the polynucleotide of the invention can comprise only a fragment of the coding region of an UP_{—}11 or OM_{—}10 polynucleotide or gene, such as a fragment of SEQ ID NO:1, SEQ ID NO:2, SEQ ID NO:3, SEQ ID NO:5, SEQ ID NO:6, SEQ ID NO:8 or SEQ ID NO:10.

When the polynucleotides of the invention are used for the recombinant production of UP_{—}11 and OM_{—}10 polypeptides, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-polypeptide sequence, or other fusion peptide portions. For example, a marker sequence which facilitates purification of the fused polypeptide can be encoded (see Gentz et al., 1989, incorporated herein by reference). The polynucleotide may also contain non-coding 5′ and 3′ sequences, such as transcribed, non-translated sequences, splicing and polyadenylation signals, ribosome binding sites and sequences that stabilize mRNA.

In addition to the GPCR nucleotide sequences shown in SEQ ID NO:1, SEQ ID NO:2, SEQ ID NO:3, SEQ ID NO:5, SEQ ID NO:6, SEQ ID NO:8 or SEQ ID NO:10, it will be appreciated by those skilled in the art that DNA sequence polymorphisms that lead to changes in the amino acid sequences of an UP_{—}11 or OM_{—}10 polypeptide may exist within a population (e.g., the human population). Such genetic polymorphism in the gene or polynucleotide may exist among individuals within a population due to natural allelic variation. As used herein, the terms “gene” and “recombinant gene” refer to polynucleotides comprising an open reading frame encoding a GPCR polypeptide, preferably a mammalian UP_{—}11 or OM_{—}10 polypeptide. Such natural allelic variations can typically result in 1-5% variance in the nucleotide sequence of the polynucleotide. Any and all such nucleotide variations and resulting amino acid polymorphisms in an UP_{—}11 or OM_{—}10 polynucleotide that are the result of natural allelic variation are intended to be within the scope of the invention. Such allelic variation includes both active allelic variants as well as non-active or reduced activity alelic variants, the later two types typically giving rise to a pathological disorder.

Moreover, nucleic acid molecules encoding UP_{—}11 or OM_{—}10 polypeptides from other species, and thus which have a nucleotide sequence which differs from the human or mouse sequence of SEQ ID NO:1, SEQ ID NO:2, SEQ ID NO:3, SEQ ID NO:5, SEQ ID NO:6, SEQ ID NO:8 or SEQ ID NO:10, are intended to be within the scope of the invention. Polynucleotides corresponding to natural allelic variants and non-human orthologues of the human UP_{—}11 or OM_{—}10 cDNA of the invention can be isolated based on their homology to the human UP_{—}11 or OM_{—}10 polynucleotides disclosed herein using the human cDNA, or a fragment thereof, as a hybridization probe according to standard hybridization techniques under stringent hybridization conditions.

Thus, a polynucleotide encoding a polypeptide of the present invention, including homologs and orthologs from species other than human, may be obtained by a process which comprises the steps of screening an appropriate library under stringent hybridization conditions with a labeled probe having the sequence of SEQ ID NO:1, SEQ ID NO:2, SEQ ID NO:3, SEQ ID NO:5, SEQ ID NO:6, SEQ ID NO:8, SEQ ID NO:10 or a fragment thereof; and isolating full-length cDNA and genomic clones containing the polynucleotide sequence. Such hybridization techniques are well known to the skilled artisan. The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low “processivity” (a measure of the ability of the enzyme to remain attached to the template during the polymerization reaction), failing to complete a DNA copy of the mRNA template during 1st strand cDNA synthesis.

Thus, in certain embodiments, the polynucleotide sequence information provided by the present invention allows for the preparation of relatively short DNA (or RNA) oligonucleotide sequences having the ability to specifically hybridize to gene sequences of the selected polynucleotides disclosed herein. The term “oligonucleotide” as used herein is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence, e.g., a sequence such as that shown in SEQ ID NO:1, SEQ ID NO:2, SEQ ID NO:3, SEQ ID NO:5, SEQ ID NO:6, SEQ ID NO:8 or SEQ ID NO:10. The ability of such nucleic acid probes to specifically hybridize to a polynucleotide encoding a GPCR lends them particular utility in a variety of embodiments. Most importantly, the probes can be used in a variety of assays for detecting the presence of complementary sequences in a given sample.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of a gene or polynucleotide that encodes a GPCR polypeptide from mammalian cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

Polynucleotides which are identical or sufficiently identical to a nucleotide sequence contained in of SEQ ID NO:1, SEQ ID NO:2, SEQ ID NO:3, SEQ ID NO:5, SEQ ID NO:6, SEQ ID NO:8, SEQ ID NO:10 or a fragment thereof, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to SEQ ID NO:1, SEQ ID NO:2, SEQ ID NO:3, SEQ ID NO:5, SEQ ID NO:6, SEQ ID NO:8, SEQ ID NO:10 or a fragment thereof. Typically these nucleotide sequences are from at least about 70% identical to at least about 95% identical to that of the reference polynucleotide sequence. The probes or primers will generally comprise at least 15 nucleotides, preferably, at least 30 nucleotides and may have at least 50 nucleotides. Particularly preferred probes will have between 30 and 50 nucleotides.

There are several methods available and well known to those skilled in the art to obtain full-length cDNAs, or extend short cDNAs, for example those based on the method of Rapid Amplification of cDNA ends (RACE) (see, Frohman et al., 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an “adaptor” sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

To provide certain of the advantages in accordance with the present invention, a preferred nucleic acid sequence employed for hybridization studies or assays includes probe molecules that are complementary to at least a 10 to 70 or so long nucleotide stretch of a polynucleotide that encodes an UP_{—}11 or OM_{—}10 polypeptide, such as that shown in SEQ ID NO:4, SEQ ID NO:7, SEQ ID NO:9 or SEQ ID NO:11. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments can be readily prepared by, for example, directly synthesizing the fragment by chemical means, by application of nucleic acid reproduction technology, such as the PCR technology of U.S. Pat. No. 4,683,202 (incorporated by reference herein in its entirety) or by excising selected DNA fragments from recombinant plasmids containing appropriate inserts and suitable restriction enzyme sites.

In another aspect, the present invention contemplates an isolated and purified polynucleotide comprising a base sequence that is identical or complementary to a segment of at least 10 contiguous bases of SEQ ID NO:1, SEQ ID NO:2, SEQ ID NO:3, SEQ ID NO:5, SEQ ID NO:6, SEQ ID NO:8 or SEQ ID NO:10, wherein the polynucleotide hybridizes to a polynucleotide that encodes an UP_{—}11 or OM_{—}10 polypeptide. Preferably, the isolated and purified polynucleotide comprises a base sequence that is identical or complementary to a segment of at least 25 to 70 contiguous bases SEQ ID NO:1, SEQ ID NO:2, SEQ ID NO:3, SEQ ID NO:5, SEQ ID NO:6, SEQ ID NO:8 or SEQ ID NO:10. For example, the polynucleotide of the invention can comprise a segment of bases identical or complementary to 40 or 55 contiguous bases of the disclosed nucleotide sequences.

Accordingly, a polynucleotide probe molecule of the invention can be used for its ability to selectively form duplex molecules with complementary stretches of the gene. Depending on the application envisioned, one will desire to employ varying conditions of hybridization to achieve varying degree of selectivity of the probe toward the target sequence. For applications requiring a high degree of selectivity, one will typically desire to employ relatively stringent conditions to form the hybrids (see Table 1).

Of course, for some applications, for example, where one desires to prepare mutants employing a mutant primer strand hybridized to an underlying template or where one seeks to isolate a GPCR polypeptide coding sequence from other cells, functional equivalents, or the like, less stringent hybridization conditions are typically needed to allow formation of the heteroduplex. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

The present invention also includes polynucleotides capable of hybridizing under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| | | Hybrid | Hybridization | Wash |
|---|---|---|---|---|
| Stringency | Polynucleotide | Length | Temperature and | Temperature |
| Condition | Hybrid | (bp)^{I} | Buffer^{H} | and BufferH |
| A | DNA:DNA | >50 | 65° C.; 1xSSC | 65° C.; |
| | | | -or- | 0.3xSSC |
| | | | 42° C.; 1xSSC, | |
| | | | 50% formamide | |
| B | DNA:DNA | <50 | T_{B}; 1xSSC | T_{B}; 1xSSC |
| C | DNA:RNA | >50 | 67° C.; 1xSSC | 67° C.; |
| | | | -or- | 0.3xSSC |
| | | | 45° C.; 1xSSC, | |
| | | | 50% formamide | |
| D | DNA:RNA | <50 | T_{D}; 1xSSC | T_{D}; 1xSSC |
| E | RNA:RNA | >50 | 70° C.; 1xSSC | 70° C.; |
| | | | -or- | 0.3xSSC |
| | | | 50° C.; 1xSSC, | |
| | | | 50% formamide | |
| F | RNA:RNA | <50 | T_{F}; 1xSSC | T_{f}; 1xSSC |
| G | DNA:DNA | >50 | 65° C.; 4xSSC | 65° C.; 1xSSC |
| | | | -or- | |
| | | | 42° C.; 4xSSC, | |
| | | | 50% formamide | |
| H | DNA:DNA | <50 | T_{H}; 4xSSC | T_{H}; 4xSSC |
| I | DNA:RNA | >50 | 67° C.; 4xSSC | 67° C.; 1xSSC |
| | | | -or- | |
| | | | 45° C.; 4xSSC, | |
| | | | 50% formamide | |
| J | DNA:RNA | <50 | T_{J}; 4xSSC | T_{J}; 4xSSC |
| K | RNA:RNA | >50 | 70° C.; 4xSSC | 67° C.; 1xSSC |
| | | | -or- | |
| | | | 50° C.; 4xSSC, | |
| | | | 50% formamide | |
| L | RNA:RNA | <50 | T_{L}; 2xSSC | T_{L}; 2xSSC |
| M | DNA:DNA | >50 | 50° C.; 4xSSC | 50° C.; 2xSSC |
| | | | -or- | |
| | | | 40° C.; 6xSSC, | |
| | | | 50% formamide | |
| N | DNA:DNA | <50 | T_{N}; 6xSSC | T_{N}; 6xSSC |
| O | DNA:RNA | >50 | 55° C.; 4xSSC | 55° C.; 2xSSC |
| | | | -or- | |
| | | | 42° C.; 6xSSC, | |
| | | | 50% formamide | |
| P | DNA:RNA | <50 | T_{P}; 6xSSC | T_{P}; 6xSSC |
| Q | RNA:RNA | >50 | 60° C.; 4xSSC | 60° C.; 2xSSC |
| | | | -or- | |
| | | | 45° C.; 6xSSC, | |
| | | | 50% formamide | |
| R | RNA:RNA | <50 | T_{R}; 4xSSC | T_{R}; 4xSSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| Buffer^{H}: SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165 M). | | | | |

Additional examples of stringency conditions for polynucleotide hybridization are provided in Sambrook et al., 1989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, eds., John Wiley & Sons, Inc., sections 2.10 and 6.3-6.4, incorporated herein by reference in its entirety.

In addition to the nucleic acid molecules encoding GPCR polypeptides described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire GPCR coding strand, or to only a fragment thereof. In one embodiment, an antisense nucleic acid molecule is antisense to a “coding region” of the coding strand of a nucleotide sequence encoding a GPCR polypeptide.

The term “coding region” refers to the region of the nucleotide sequence comprising codons which are translated into amino acid residues, e.g., the entire coding region of SEQ ID NO:1, SEQ ID NO:2, SEQ ID NO:3, SEQ ID NO:5, SEQ ID NO:6, SEQ ID NO:8 or SEQ ID NO:10, comprises nucleotides 298 to 1,653, 1 to 1,313, 671 to 2,206, 684 to 2,033, 685 to 2,034, 332 to 1,858 or 250 to 1,785, respectively. In another embodiment, the antisense nucleic acid molecule is antisense to a “noncoding region” of the coding strand of a nucleotide sequence encoding a GPCR polypeptide. The term “noncoding region” refers to 5′ and 3′ sequences which flank the coding region that are not translated into amino acids (i.e., also referred to as 5′ and 3′ untranslated regions). For example, noncoding regions of SEQ ID NO:1 comprise nucleotides 1 to 297 and 1,654 to 3,824, noncoding regions of SEQ ID NO:2 comprise nucleotides 1,314 to 3,546, noncoding regions of SEQ ID NO:3 comprise nucleotides 1 to 670 and 2,027 to 3,779, noncoding regions of SEQ ID NO:5 comprise nucleotides 1 to 683 and 2,034 to 3,384, noncoding regions of SEQ ID NO:6 comprise nucleotides 1 to 684 and 2,035 to 3,384, noncoding regions of SEQ ID NO:8 comprise nucleotides 1 to 331 and 1,859 to 4,718 and noncoding regions of SEQ ID NO:10 comprise nucleotides 1 to 249 and 1,786 to 5,386.

Given the coding strand sequence encoding the GPCR polypeptides disclosed herein (e.g., SEQ ID NO:1, SEQ ID NO:2, SEQ ID NO:3, SEQ ID NO:5, SEQ ID NO:6, SEQ ID NO:8 or SEQ ID NO:10), antisense nucleic acids of the invention can be designed according to the rules of Watson and Crick base pairing. The antisense nucleic acid molecule can be complementary to the entire coding region of UP_{—}11 or OM_{—}10 mRNA, but more preferably is an oligonucleotide which is antisense to only a fragment of the coding or noncoding region of UP_{—}11 or OM_{—}10 mRNA. For example, the antisense oligonucleotide can be complementary to the region surrounding the translation start site of UP_{—}11 mRNA.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, I-methylguanine, I-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an UP_{—}11 or OM_{—}10 polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual γ-units, the strands run parallel to each other (Gaultier et al., 1987). The antisense nucleic acid molecule can also comprise a 2′-o-methylribonucleotide (Inoue et al., 1987(a)) or a chimeric RNA-DNA analogue (Inoue et al., 1987(b)).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988)) can be used to catalytically cleave GPCR mRNA transcripts to thereby inhibit translation of GPCR mRNA. A ribozyme having specificity for a GPCR-encoding nucleic acid can be designed based upon the nucleotide sequence of a GPCR cDNA disclosed herein (e.g., SEQ ID NO:I). For example, a derivative of a Tetrahymena L-19 IVS RNA can be constructed in which the nucleotide sequence of the active site is complementary to the nucleotide sequence to be cleaved in a GPCR-encoding mRNA. See, e.g., Cech et al. U.S. Pat. No. 4,987,071 and Cech et al. U.S. Pat. No. 5,116,742, both of which are incorporated by reference herein in their entirety. Alternatively, GPCR mRNA can be used to select a catalytic RNA having a specific ribonuclease activity from a pool of RNA molecules. See, e.g., Bartel and Szostak, 1993.

Alternatively gene expression can be inhibited by targeting nucleotide sequences complementary to the regulatory region of the UP_{—}11 or OM_{—}10 gene (e.g., the gene promoter and/or enhancers) to form triple helical structures that prevent transcription of the gene in target cells. See generally, Helene, 1991; Helene et al., 1992; and Maher, 1992).

GPCR gene expression can also be inhibited using RNA interference (RNAi). This is a technique for post-transcriptional gene silencing (PTGS), in which target gene activity is specifically abolished with cognate double-stranded RNA (dsRNA). RNAi resembles in many aspects PTGS in plants and has been detected in many invertebrates including trypanosome, hydra, planaria, nematode and fruit fly (Drosophila melangnoster). It may be involved in the modulation of transposable element mobilization and antiviral state formation. RNAi in mammalian systems is disclosed in International Application No. WO 00/63364 which is incorporated by reference herein in its entirety. Basically, dsRNA of at least about 600 nucleotides, homologous to the target (GPCR) is introduced into the cell and a sequence specific reduction in gene activity is observed.

B. Isolated UP_{—}11 and OM_{—}10 Polypeptides

In particular embodiments, the present invention provides isolated and purified UP_{—}11 and OM_{—}10 GPCR polypeptides. Preferably, a GPCR polypeptide of the invention is a recombinant polypeptide. Typically, a GPCR is produced by recombinant expression in a non-human cell.

An UP_{—}11 polypeptide according to the present invention encompasses a polypeptide that comprises: 1) the amino acid sequence shown in SEQ ID NO:4 or SEQ ID NO:7; 2) functional and non-functional naturally occurring allelic variants of human UP_{—}11 polypeptide; 3) recombinantly produced variants of human UP_{—}11 polypeptide; and 4) UP_{—}11 polypeptides isolated from organisms other than humans (orthologues of human UP_{—}11 polypeptide).

An OM_{—}10 polypeptide according to the present invention encompasses a polypeptide that comprises: 1) the amino acid sequence shown in SEQ ID NO:9 or SEQ ID NO:11; 2) functional and non-functional naturally occurring allelic variants of human OM_{—}10 polypeptide; 3) recombinantly produced variants of human OM_{—}10 polypeptide; and 4) OM_{—}10 polypeptides isolated from organisms other than humans (orthologues of human OM_{—}10 polypeptide).

An allelic variant of the human UP_{—}11 polypeptide according to the present invention encompasses 1) a polypeptide isolated from human cells or tissues; 2) a polypeptide encoded by the same genetic locus as that encoding the human UP_{—}11 polypeptide; and 3) a polypeptide that contains substantial homology to a human UP_{—}11. Similarly, an allelic variant of the human OM_{—}10 polypeptide according to the present invention encompasses 1) a polypeptide isolated from human cells or tissues; 2) a polypeptide encoded by the same genetic locus as that encoding the human OM_{—}10 polypeptide; and 3) a polypeptide that contains substantially homology to a human OM_{—}10.

Allelic variants of human UP_{—}11 and OM_{—}10 include both functional and non-functional UP_{—}11 and OM_{—}10 polypeptides. Functional allelic variants are naturally occurring amino acid sequence variants of the human UP_{—}11 or OM_{—}10 polypeptide that maintain the ability to bind an UP_{—}11 or OM_{—}10 ligand and transduce a signal within a cell. Functional allelic variants will typically contain only conservative substitution of one or more amino acids of SEQ ID NO:4, SEQ ID NO:7, SEQ ID NO:9 or SEQ ID NO:11 or substitution, deletion or insertion of non-critical residues in non-critical regions of the polypeptide.

Non-functional allelic variants are naturally occurring amino acid sequence variants of human UP_{—}11 or OM_{—}10 polypeptide that do not have the ability to either bind ligand and/or transduce a signal within a cell. Non-functional allelic variants will typically contain a non-conservative substitution, a deletion, or insertion or premature truncation of the amino acid sequence of SEQ ID NO:4, SEQ ID NO:7, SEQ ID NO:9 or SEQ ID NO:11 or a substitution, insertion or deletion in critical residues or critical regions.

The present invention further provides non-human orthologues of human UP_{—}11 or OM_{—}10 polypeptide. Orthologues of human UP_{—}11 or OM_{—}10 polypeptide are polypeptides that are isolated from non-human organisms and possess the same ligand binding and signaling capabilities of the human GPCR polypeptide. Orthologues of the human UP_{—}11 or OM_{—}10 polypeptide can readily be identified as comprising an amino acid sequence that is substantially homologous to SEQ ID NO:4, SEQ ID NO:7, SEQ ID NO:9 or SEQ ID NO:11.

As used herein, two proteins are substantially homologous when the amino acid sequence of the two proteins (or a region of the proteins) are at least about 60-65%, typically at least about 70-75%, more typically at least about 80-85%, and most typically at least about 90-95% or more homologous to each other. To determine the percent homology of two amino acid sequences (e.g., SEQ ID NO:4 and an allelic variant thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., SEQ ID NO:4) is occupied by the same amino acid residue or nucleotide as the corresponding position in the other sequence (e.g., an allelic variant of the human UP_{—}11 protein), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=number of identical positions/total number of positions×100).

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith and Waterman, 1981, by the homology alignment algorithm of Needleman and Wunsch, 1970, by the search for similarity method of Pearson and Lipman, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., John Wiley & Sons: 1992).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng and Doolittle, 1987. The method used is similar to the method described by Higgins and Sharp, 1989. The program can align up to 300 sequences, each of a maximum length of 5,000 nucleotides or amino acids. The multiple alignment procedure begins with the pairwise alignment of the-two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased.

Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, 1989) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Modifications and changes can be made in the structure of a polypeptide of the present invention and still obtain a molecule having UP_{—}11 or OM_{—}10 like receptor characteristics. For example, certain amino acids can be substituted for other amino acids in a sequence without appreciable loss of receptor activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated by reference herein in its entirety, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (see Table 2). The present invention thus contemplates functional or biological equivalents of a GPCR polypeptide as set forth above.

TABLE 2

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single stranded and double stranded form. Typically, site-directed mutagenesis in accordance herewith is performed by first obtaining a single-stranded vector which includes within its sequence a DNA sequence which encodes all or a portion of the GPCR polypeptide sequence selected. An oligonucleotide primer bearing the desired mutated sequence is prepared (e.g., synthetically). This primer is then annealed to the single-stranded vector, and extended by the use of enzymes such as E coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

The UP_{—}11 and OM_{—}10 GPCR polypeptide is a GPCR that participates in signaling pathways within cells. As used herein, a signaling pathway refers to the modulation (e.g., stimulated or inhibited) of a cellular function/activity upon the binding of a ligand to the GPCR polypeptide. Examples of such functions include mobilization of intracellular molecules that participate in a signal transduction pathway, e.g., phosphatidylinositol 4,5-bisphosphate (PIP₂), inositol 1,4,5-triphosphate (IP₃) or adenylate cyclase; polarization of the plasma membrane; production or secretion of molecules; alteration in the structure of a cellular component; cell proliferation, e.g., synthesis of DNA; cell migration; cell differentiation; and cell survival.

Depending on the type of cell, the response mediated by the GPCR polypeptide/ligand binding may be different. For example, in some cells, binding of a ligand to a GPCR polypeptide may stimulate an activity such as adhesion, migration, differentiation, etc. through phosphatidylinositol or cyclic AMP metabolism and turnover while in other cells, the binding of the ligand to the GPCR polypeptide will produce a different result. Regardless of the cellular activity modulated by GPCR, it is universal that the GPCR polypeptide is a GPCR and interacts with a “G-polypeptide” to produce one or more secondary signals in a variety of intracellular signal transduction pathways, e.g., through phosphatidylinositol or cyclic AMP metabolism and turnover, in a cell. G-polypeptides represent a family of heterotrimeric polypeptides composed of α, β and γ subunits, which bind guanine nucleotides. These polypeptides are usually linked to cell surface receptors, e.g., receptors containing seven transmembrane domains, such as the ligand receptors. Following ligand binding to the receptor, a conformational change is transmitted to the G-polypeptide, which causes the α-subunit to exchange a bound GDP molecule for a GTP molecule and to dissociate from the N-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits.

As used herein, “phosphatidylinositol turnover and metabolism” refers to the molecules involved in the turnover and metabolism of phosphatidylinositol 4,5-bisphosphate (PIP₂) as well as to the activities of these molecules. PIP₂ is a phospholipid found in the cytosolic leaflet of the plasma membrane. Binding of a ligand to the GPCR activates, in some cells, the plasma-membrane enzyme phospholipase C that in turn can hydrolyze PIP₂ to produce 1,2-diacylglycerol (DAG) and inositol 1,4,5-triphosphate (IP₃). Once formed IP₃ can diffuse to the endoplasmic reticulum surface where it can bind an IP₃ receptor, e.g., a calcium channel polypeptide containing an IP₃ binding site. IP₃ binding can induce opening of the channel, allowing calcium ions to be released into the cytoplasm. IP₃ can also be phosphorylated by a specific kinase to form inositol 1,3,4,5-tetraphosphate, a molecule which can cause calcium entry into the cytoplasm from the extracellular medium. IP₃ and 1,3,4,5-tetraphosphate can subsequently be hydrolyzed very rapidly to the inactive products inositol 1,4-biphosphate and inositol 1,3,4-triphosphate, respectively. These inactive products can be recycled by the cell to synthesize PIP₂. The other second messenger produced by the hydrolysis of PIP₂, namely 1,2-diacylglycerol (DAG), remains in the cell membrane where it can serve to activate the enzyme polypeptide kinase C. Polypeptide kinase C is usually found soluble in the cytoplasm of the cell, but upon an increase in the intracellular calcium concentration, this enzyme can move to the plasma membrane where it can be activated by DAG. The activation of polypeptide kinase C in different cells results in various cellular responses such as the phosphorylation of glycogen synthase, or the phosphorylation of various transcription factors, e.g., NF-kB. The language “phosphatidylinositol activity,” as used herein, refers to an activity of PIP₂ or one of its metabolites.

Another signaling pathway in which the GPCR polypeptide may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand induced stimulation of certain G-polypeptide coupled receptors. In the ligand signaling pathway, binding of ligand to a ligand receptor can lead to the activation of the enzyme adenylate cyclase, which catalyzes the synthesis of cAMP. The newly synthesized cAMP can in turn activate a cAMP-dependent polypeptide kinase. This activated kinase can, for example, phosphorylate a voltage-gated potassium channel polypeptide, or an associated polypeptide, and lead to the inability of the potassium channel to open during an action potential. The inability of the potassium channel to open results in a decrease in the outward flow of potassium, which normally repolarizes the membrane of a neuron, leading to prolonged membrane depolarization. Of course, the activated cAMP kinase can affect other molecules as well, such as enzymes (e.g., metabolic enzymes), transcription factors, adenylyl cyclase and the like.

An UP_{—}11 or OM_{—}10 receptor polypeptide of the present invention is understood to be any GPCR polypeptide comprising substantial sequence similarity, structural similarity and/or functional similarity to an UP_{—}11 or OM_{—}10 polypeptide comprising the amino acid sequence selected from the group consisting of SEQ ID NO:4, SEQ ID NO:7, SEQ ID NO:9 and SEQ ID NO:11. In addition, an UP_{—}11 or OM_{—}10 polypeptide of the invention is not limited to a particular source. Thus, the invention provides for the general detection and isolation of the genus of UP_{—}11 or OM_{—}10 receptor polypeptides from a variety of sources. For example, GPCR polypeptides are found in virtually all mammals including human. The sequence of GP57 and GP58 receptors have been previously described (Lee et al., 2000; European Application No. EP 0859055). As is the case with other receptors, there is likely little variation between the structure and function of GPCR receptors in different species. Where there is a difference between species, identification of those differences is well within the skill of an artisan. Thus, the present invention contemplates an UP_{—}11 or OM_{—}10 polypeptide from any mammal, wherein the preferred mammal is a human.

The invention further provides fragments of UP_{—}11 or OM_{—}10 polypeptides. As used herein, a fragment comprises at least 8 contiguous amino acids from UP_{—}11 or OM_{—}10. It is contemplated in the present invention, that an UP_{—}11 or OM_{—}10 polypeptide may advantageously be cleaved into fragments for use in further structural or functional analysis, or in the generation of reagents such as UP_{—}11 or OM_{—}10 related polypeptides and UP_{—}11, OM_{—}10-specific antibodies. This can be accomplished by treating purified or unpurified UP_{—}11 or OM_{—}10 with a peptidase such as endopolypeptidease glu-C (Boehringer, Indianapolis, Ind.). Treatment with CNBr is another method by which UP_{—}11 or OM_{—}10 fragments may be produced from natural UP_{—}11 or OM_{—}10. Recombinant techniques also can be used to produce specific fragments of UP_{—}11 or OM_{—}10.

Preferred fragments are fragments that possess one or more of the biological activities of the UP_{—}11 or OM_{—}10 polypeptide, for example the ability to bind to a G-protein, as well as fragments that can be used as an immunogen to generate anti-UP_{—}11 or anti-OM_{—}10 antibodies. Biologically active fragments of the UP_{—}11 or OM_{—}10 polypeptide include peptides comprising amino acid sequences derived from the amino acid sequence of an UP_{—}11 or OM_{—}10 polypeptide, e.g., the amino acid sequence shown in SEQ ID NO:4, SEQ ID NO:7, SEQ ID NO:9, SEQ ID NO:11 or the amino acid sequence of a polypeptide homologous to the UP_{—}11 or OM_{—}10 polypeptide, which include less amino acids than the full length UP_{—}11 or OM_{—}10 polypeptide or the full length polypeptide which is homologous to the UP_{—}11 or OM_{—}10 polypeptide, and exhibit at least one activity of the UP_{—}11 or OM_{—}10 polypeptide. Typically, biologically active fragments (peptides, e.g., peptides which are, for example, 5, 10, 15, 20, 30, 35, 36, 37, 38, 39, 40, 50, 100 or more amino acids in length) comprise a domain or motif, e.g., a transmembrane domain or G-protein binding domain.

In addition, the invention also contemplates that compounds sterically similar to an UP_{—}11 or OM_{—}10 may be formulated to mimic the key portions of the peptide structure, called peptidomimetics. Mimetics are peptide-containing molecules which mimic elements of polypeptide secondary structure. See, for example, Johnson et al. (1993). The underlying rationale behind the use of peptide mimetics is that the peptide backbone of polypeptides exists chiefly to orient amino acid side chains in such a way as to facilitate molecular interactions, such as those of receptor and ligand.

Successful applications of the peptide mimetic concept have thus far focused on mimetics of β-turns within polypeptides. Likely β-turn structures within UP_{—}11 or OM_{—}10 can be predicted by computer-based algorithms as discussed above. Once the component amino acids of the turn are determined, mimetics can be constructed to achieve a similar spatial orientation of the essential elements of the amino acid side chains.

The isolated UP_{—}11 or OM_{—}10 polypeptides can be purified from cells that naturally express the polypeptide, purified from cells that have been altered to express the UP_{—}11 or OM_{—}10 polypeptide, or synthesized using known protein synthesis methods. Preferably, as described below, the isolated UP_{—}11 or OM_{—}10 polypeptide is produced by recombinant DNA techniques. For example, a nucleic acid molecule encoding the protein is cloned into an expression vector, the expression vector is introduced into a host cell and the UP_{—}11 or OM_{—}10 polypeptide is expressed in the host cell. The UP_{—}11 or OM_{—}10 polypeptide can then be isolated from the cells by an appropriate purification scheme using standard protein purification techniques. As an alternative to recombinant expression, the UP_{—}11 or OM_{—}10 polypeptide or fragment can be synthesized chemically using standard peptide synthesis techniques. Lastly, native UP_{—}11 or OM_{—}10 polypeptide can be isolated from cells that naturally express the UP_{—}11 or OM_{—}10 polypeptide (e.g., caudate nucleus, putatem).

The present invention further provides UP_{—}11 or OM_{—}10 chimeric or fusion proteins. As used herein, an UP_{—}11 or OM_{—}10 polypeptide “chimeric protein” or “fusion protein” comprises an UP_{—}11 or OM_{—}10 polypeptide operatively linked to a non-UP_{—}11 or OM_{—}10 polypeptide. An “UP_{—}11 or OM_{—}10 polypeptide” refers to a polypeptide having an amino acid sequence corresponding to an UP_{—}11 or OM_{—}10 polypeptide, whereas a “non-UP_{—}11 or OM_{—}10 polypeptide” refers to a heterologous polypeptide having an amino acid sequence corresponding to a polypeptide which is not substantially homologous to the UP_{—}11 or OM_{—}10 polypeptide, e.g., a protein which is different from the UP_{—}11 or OM_{—}10 polypeptide. Within the context of fusion proteins, the term “operatively linked” is intended to indicate that the UP_{—}11 or OM_{—}10 polypeptide and the non-UP_{—}11 or OM_{—}10 polypeptide are fused in-frame to each other. The non-UP_{—}11 or OM_{—}10 polypeptide can be fused to the N-terminus or C-terminus of the UP_{—}11 or OM_{—}10 polypeptide. For example, in one embodiment the fusion polypeptide is a GST-UP_{—}11 or OM_{—}10 fusion polypeptide in which the UP_{—}11 or OM_{—}10 sequences are fused to the C-terminus of the GST sequences. Other types of fusion polypeptides include, but are not limited to, enzymatic fusion proteins, for example β-galactosidase fusions, yeast two-hybrid GAL fusions, poly-His fusions and Ig fusions.

Such fusion polypeptides, particularly poly-His fusions, can facilitate the purification of recombinant UP_{—}11 or OM_{—}10 polypeptide. In another embodiment, the fusion protein is an UP_{—}11 or OM_{—}10 polypeptide containing a heterologous signal sequence at its N-terminus. In certain host cells (e.g., mammalian host cells), expression and/or secretion of an UP_{—}11 or OM_{—}10 polypeptide can be increased by using a heterologous signal sequence.

Preferably, an UP_{—}11 or OM_{—}10 chimeric or fusion protein is produced by standard recombinant DNA techniques. For example, DNA fragments coding for the different protein sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST protein). An UP_{—}11 or OM_{—}10-encoding nucleic acid can be cloned into such an expression vector such that the fusion moiety is linked in-frame to the UP_{—}11 or OM_{—}10 polypeptide.

C. Anti-UP_{—}11 and Anti-OM_{—}10 Antibodies

In another embodiment, the present invention provides antibodies immunoreactive with UP_{—}11 and OM_{—}10 polypeptides. Preferably, the antibodies of the invention are monoclonal antibodies. Additionally, the UP_{—}11 and OM_{—}10 polypeptides comprise the amino acid residue sequence of SEQ ID NO:4, SEQ ID NO:7, SEQ ID NO:9, or SEQ ID NO:11. In certain embodiments, human OM_{—}10 polypeptide fragments comprising amino acid sequences of SEQ ID NOs:12-16 are used to generate monoclonal and/or polyclonal antisera. Similarly, human UP_{—}11 polypeptide fragments comprising amino acid sequences of SEQ ID Nos: 17-21 are used to generate monoclonal and/or polyclonal sera. Means for preparing and characterizing antibodies are well known in the art (see, e.g., Antibodies “A Laboratory Manual, E. Howell and D. Lane, Cold Spring Harbor Laboratory, 1988). In yet other embodiments, the present invention provides antibodies immunoreactive with UP_{—}11 and OM_{—}10 polynucleotides.

As used herein, an antibody is said to selectively bind to an UP_{—}11 or OM_{—}10 polypeptide when the antibody binds to UP_{—}11 or OM_{—}10 polypeptides and does not selectively bind to unrelated proteins. A skilled artisan will readily recognize that an antibody may be considered to substantially bind an UP_{—}11 or OM_{—}10 polypeptide even if it binds to proteins that share homology with a fragment or domain of the UP_{—}11 or OM_{—}10 polypeptide.

The term “antibody” as used herein refers to immunoglobulin molecules and immunologically active fragments of immunoglobulin molecules, i.e., molecules that contain an antigen binding site which specifically binds (immunoreacts with) an antigen, such as UP_{—}11 or OM_{—}10. Examples of immunologically active fragments of immunoglobulin molecules include F(ab) and F(ab′)₂ fragments which can be generated by treating the antibody with an enzyme such as pepsin. The invention provides polyclonal and monoclonal antibodies that bind UP_{—}11 or OM_{—}10. The term “monoclonal antibody” or “monoclonal antibody composition,” as used herein, refers to a population of antibody molecules that contain only one species of an antigen binding site capable of immunoreacting with a particular epitope of UP_{—}11 or OM_{—}10. A monoclonal antibody composition thus typically displays a single binding affinity for a particular UP_{—}11 or OM_{—}10 polypeptide with which it immunoreacts.

To generate anti-UP_{—}11 or OM_{—}10 antibodies, an isolated UP_{—}11 or OM_{—}10 polypeptide, or a fragment thereof, is used as an immunogen to generate antibodies that bind UP_{—}11 or OM_{—}10 using standard techniques for polyclonal and monoclonal antibody preparation. The full-length UP_{—}11 or OM_{—}10 polypeptide can be used or, alternatively, an antigenic peptide fragment of UP_{—}11 or OM_{—}10 can be used as an immunogen. An antigenic fragment of the UP_{—}11 or OM_{—}10 polypeptide will typically comprises at least 8 contiguous amino acid residues of an UP_{—}11 or OM_{—}10 polypeptide, e.g., 8 contiguous amino acids from SEQ ID NO:4, SEQ ID NO:7, SEQ ID NO:9 or SEQ ID NO:11. Preferably, the antigenic peptide comprises at least 10 amino acid residues, more preferably at least 15 amino acid residues, even more preferably at least 20 amino acid residues, and most preferably at least 30 amino acid residues of an UP_{—}11 or OM_{—}10 polypeptide. Preferred fragments for generating anti-UP_{—}11 or OM_{—}10 antibodies are regions of UP_{—}11 or OM_{—}10 polypeptide that are located on the surface of the polypeptide, e.g., hydrophilic regions.

An UP_{—}11 or OM_{—}10 immunogen typically is used to prepare antibodies by immunizing a suitable subject, (e.g., rabbit, goat, mouse or other mammal, chicken) with the immunogen. An appropriate immunogenic preparation can contain, for example, recombinantly expressed UP_{—}11 or OM_{—}10 polypeptide or a chemically synthesized UP_{—}11 or OM_{—}10 peptide. The preparation can further include an adjuvant, such as Freund's complete or incomplete adjuvant, or similar immunostimulatory agent. Immunization of a suitable subject with an immunogenic UP_{—}11 or OM_{—}10 preparation induces a polyclonal anti-UP_{—}11 or OM_{—}10 antibody response.

Polyclonal anti-UP_{—}11 or OM_{—}10 antibodies can be prepared as described above by immunizing a suitable subject with an UP_{—}11 or OM_{—}10 immunogen. Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e.g., a polypeptide or polynucleotide) of the present invention with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier polypeptide are well known in the art and include glutaraldehyde, m-maleimidobencoyl-N-hydroxysuccinimide ester, carbodiimide and bis-biazotized benzidine.

As is also well known in the art, immunogencity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used for the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

In another aspect, the present invention contemplates a process of producing an antibody immunoreactive with a GPCR polypeptide comprising the steps of (a) transfecting recombinant host cells with a polynucleotide that encodes an UP_{—}11 or OM_{—}10 polypeptide; (b) culturing the host cells under conditions sufficient for expression of the polypeptide; (c) recovering the polypeptides; and (d) preparing the antibodies to the polypeptides. Preferably, the host cell is transfected with the polynucleotide of SEQ ID NO:1, SEQ ID NO:2, SEQ ID NO:3, SEQ ID NO:5, SEQ ID NO:6, SEQ ID NO:8 or SEQ ID NO:10. Even more preferably, the present invention provides antibodies prepared according to the process described above.

A monoclonal antibody of the present invention can be readily prepared through use of well-known techniques such as those exemplified in U.S. Pat. No. 4,196,265, herein incorporated by reference. Typically, a technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, e.g., by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptide. The selected clones can then be propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides and polynucleotide of the invention can be recognized as antigens, and thus identified. Once identified, those polypeptides and polynucleotide can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

Additionally, examples of methods and reagents particularly amenable for use in generating and screening antibody display library can be found in, for example, U.S. Pat. No. 5,223,409; International Application No. WO 92/18619; International Application No. WO 91/17271; International Application No. WO 92/20791; International Application No. WO 92/15679; International Application No. WO 93/01288; International Application No. WO 92/01047; International Application No. WO 92/09690 and International Application No. WO 90/02809.

Additionally, recombinant anti-UP_{—}11 or OM_{—}10 antibodies, such as chimeric and humanized monoclonal antibodies, comprising both human and non-human fragments, which can be made using standard recombinant DNA techniques, are within the scope of the invention. Such chimeric and humanized monoclonal antibodies can be produced by recombinant DNA techniques known in the art, for example using methods described in U.S. Pat. No. 6,054,297, European Application Nos. EP 184,187; EP 171,496; EP 173,494; International Application No. WO 86/01533; U.S. Pat. No. 4,816,567; and European Application No. EP 125,023.

An anti-UP_{—}11 or OM_{—}10 polypeptide antibody (e.g., monoclonal antibody) can be used to isolate UP_{—}11 or OM_{—}10 polypeptides by standard techniques, such as affinity chromatography or immunoprecipitation.

An anti-UP_{—}11 or OM_{—}10 polypeptide antibody can facilitate the purification of a natural UP_{—}11 or OM_{—}10 polypeptides from cells and recombinantly produced UP_{—}11 or OM_{—}10 polypeptide expressed in host cells. Moreover, an anti-UP_{—}11 or OM_{—}10 polypeptide antibody can be used to detect UP_{—}11 or OM_{—}10 polypeptide (e.g., in a cellular lysate or cell supernatant) in order to evaluate the abundance and pattern of expression of the UP_{—}11 or OM_{—}10 polypeptide. The detection of circulating fragments of an UP_{—}11 or OM_{—}10 polypeptide can be used to identify UP_{—}11 or OM_{—}10 polypeptide turnover in a subject. Anti-UP_{—}11 or OM_{—}10 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ¹⁵S or ³H.

D. Recombinant Expression Vectors and Host Cells

In another embodiment, the present invention provides expression vectors comprising polynucleotides that encode UP_{—}11 or OM_{—}10 polypeptides. Preferably, the expression vectors of the present invention comprise polynucleotides that encode polypeptides comprising the amino acid residue sequence of SEQ ID NO:4, SEQ ID NO:7, SEQ ID NO:9 or SEQ ID NO: 11. More preferably, the expression vectors of the present invention comprise polynucleotides comprising the nucleotide base sequence of SEQ ID NO:1, SEQ ID NO:2, SEQ ID NO:3, SEQ ID NO:5, SEQ ID NO:6, SEQ ID NO:8 or SEQ ID NO:10. Even more preferably, the expression vectors of the invention comprise polynucleotides operatively linked to an enhancer-promoter. In certain embodiments, the expression vectors of the invention comprise polynucleotides operatively linked to a prokaryotic promoter. Alternatively, the expression vectors of the present invention comprise polynucleotides operatively linked to an enhancer-promoter that is a eukaryotic promoter, and the expression vectors further comprise a polyadenylation signal that is positioned 3′ of the carboxy-terminal amino acid and within a transcriptional unit of the encoded polypeptide.

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Expression of proteins in prokaryotes is most often carried out in E. Coli with vectors containing constitutive or inducible promoters directing the expression of either fusion or non-fusion proteins. Fusion vectors add a number of amino acids to a protein encoded therein, to the amino or carboxy terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson, 1988), pMAL (New England Biolabs, Beverly; Mass.) and pRIT5 (Pharmacia, Piscataway, N.J.) which fuse glutathione S-transferase (GST), maltose E binding protein, or protein A, respectively, to the target recombinant protein.

In one embodiment, the coding sequence of the UP_{—}11 or OM_{—}10 gene is cloned into a pGEX expression vector to create a vector encoding a fusion protein comprising, from the N-terminus to the C-terminus, GST-thrombin cleavage site-UP_{—}11 or OM_{—}10 polypeptide. The fusion protein can be purified by affinity chromatography using glutathione-agarose resin. Recombinant UP_{—}11 or OM_{—}10 polypeptides unfused to GST can be recovered by cleavage of the fusion protein with thrombin.

Examples of suitable inducible non-fusion E coli expression vectors include pTrc (Amann et al., 1988) and pET IId (Studier et al., 1990). Target gene expression from the pTrc vector relies on host RNA polymerase transcription from a hybrid trp-lac fusion promoter. Target gene expression from the pET IId vector relies on transcription from a T7 gn1 0-lac fusion promoter mediated by a coexpressed viral RNA polymerase J7 gnl. This viral polymerase is supplied by host strains BL21 (DE3) or HMS I 74(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E coli. Such alteration of nucleic acid sequences of the invention can be carried out by standard DNA mutagenesis or synthesis techniques.

In another embodiment, the UP_{—}11 or OM_{—}10 polynucleotide expression vector is a yeast expression vector. Examples of vectors for expression in yeast S. cerivisae include pYepSec I (Baldari, et al., 1987), pMFa (Kurjan and Herskowitz, 1982), pJRY88 (Schultz et al., 1987), and pYES2 (Invitrogen Corporation, San Diego, Calif.).

Alternatively, an UP_{—}11 or OM_{—}10 polynucleotide can be expressed in insect cells using, for example, baculovirus expression vectors. Baculovirus vectors available for expression of proteins in cultured insect cells (e.g., Sf 9 cells) include the pAc series (Smith et al., 1983) and the pVL series (Lucklow and Summers, 1989).

In yet another embodiment, a polynucleotide of the invention is expressed in mammalian cells using a mammalian expression vector. Examples of mammalian expression vectors include pCDM8 (Seed, 1987) and pMT2PC (Kaufman et al., 1987). When used in mammalian cells, the expression vector's control functions are often provided by viral regulatory elements.

For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., “Molecular Cloning: A Laboratory Manual” 2nd ed, Cold Spring Harbor Laboratory, Cold Spring Harbor Laboratory Press, Cold Spring Harbor, N.Y., 1989, incorporated herein by reference in its entirety.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non limiting examples of suitable tissue-specific promoters include the albumin promoter (liver-specific; Pinkert et al., 1987), lymphoid-specific promoters (Calame and Eaton, 1988), in particular promoters of T cell receptors (Winoto and Baltimore, 1989) and immunoglobulins (Banerji et al., 1983, Queen and Baltimore, 1983), neuron-specific promoters (e.g., the neurofilament promoter; Byrne and Ruddle, 1989), pancreas-specific promoters (Edlund et al., 1985), and mammary gland-specific promoters (e.g., milk whey promoter; U.S. Pat. No. 4,873,316 and European Application No. EP 264,166). Developmentally-regulated promoters are also encompassed, for example the murine hox promoters (Kessel and Gruss, 1990) and the α-fetoprotein promoter (Campes and Tilghman, 1989).

The present invention also relates to improved methods for both the in vitro production of UP_{—}11 or OM_{—}10 polypeptides and for the production and delivery of UP_{—}11 or OM_{—}10 polypeptides by gene therapy. The present invention includes approaches which activate expression of endogenous cellular genes, and further allows amplification of the activated endogenous cellular genes, which does not require in vitro manipulation and transfection of exogenous DNA encoding UP_{—}11 or OM_{—}10 polypeptides. These methods are described in PCT International Application WO 94/12650, U.S. Pat. No. 5,968,502, and Harrington et al., 2001, all of which are incorporated in their entirety by reference. These, and variations of them which one skilled in the art will recognize as equivalent, may collectively be referred to as “gene activation”.

Thus, in certain embodiments, the invention relates to transfected cells, both transfected primary or secondary cells (i.e., non-immortalized cells) and transfected immortalized cells, useful for producing proteins, methods of making such cells, methods of using the cells for in vitro protein production and methods of gene therapy. Cells of the present invention are of vertebrate origin, particularly of mammalian origin and even more particularly of human origin. Cells produced by the method of the present invention contain exogenous DNA which encodes a therapeutic product, exogenous DNA which is itself a therapeutic product and/or exogenous DNA which causes the transfected cells to express a gene at a higher level or with a pattern of regulation or induction that is different than occurs in the corresponding nontransfected cell.

The present invention also relates to methods by which primary, secondary, and immortalized cells are transfected to include exogenous genetic material, methods of producing clonal cell strains or heterogeneous cell strains, and methods of immunizing animals, or producing antibodies in immunized animals, using the transfected primary, secondary, or immortalized cells.

The present invention relates particularly to a method of gene targeting or homologous recombination in cells of vertebrate, particularly mammalian, origin. That is, it relates to a method of introducing DNA into primary, secondary, or immortalized cells of vertebrate origin through homologous recombination, such that the DNA is introduced into genomic DNA of the primary, secondary, or immortalized cells at a pre-selected site. The targeting sequences used are determined by (selected with reference to) the site into which the exogenous DNA is to be inserted. The cDNA UP_{—}11 or OM_{—}10 sequences provided by the present invention (i.e., SEQ ID NO:1, SEQ ID NO:2, SEQ ID NO:3, SEQ ID NO:5, SEQ ID NO:6, SEQ ID NO:8, SEQ ID NO:10) are useful in these methods. The present invention further relates to homologously recombinant primary, secondary, or immortalized cells, referred to as homologously recombinant (HR) primary, secondary or immortalized cells, produced by the present method and to uses of the HR primary, secondary, or immortalized cells.

The present invention also relates to a method of activating (ie., turning on) an UP_{—}11 or OM_{—}10 gene present in primary, secondary, or immortalized cells of vertebrate origin, which is normally not expressed in the cells or is not expressed at physiologically significant levels in the cells as obtained. According to the present method, homologous recombination is used to replace or disable the regulatory region normally associated with the gene in cells as obtained with a regulatory sequence which causes the gene to be expressed at levels higher than evident in the corresponding nontransfected cell, or to display a pattern of regulation or induction that is different than evident in the corresponding nontransfected cell. The present invention, therefore, relates to a method of making proteins by turning on or activating an endogenous gene which encodes the desired product in transfected primary, secondary, or immortalized cells.

In one embodiment, the activated gene can be further amplified by the inclusion of a selectable marker gene which has the property that cells containing amplified copies of the selectable marker gene can be selected for by culturing the cells in the presence of the appropriate selectable agent. The activated endogenous gene which is near or linked to the amplified selectable marker gene will also be amplified in cells containing the amplified selectable marker gene. Cells containing many copies of the activated endogenous gene are useful for in vitro protein production and gene therapy.

In certain embodiments, the present invention relates also to methods for activating the expression of an endogenous gene in a cell or over-expressing an endogenous gene in a cell by non-homologous or random activation of gene expression (RAGE). The method comprises introducing a vector into the cell, allowing the vector to integrate into the genome of the cell by non-homologous recombination and allowing activation or over-expression of the endogenous gene in the cell. The use of non-homologous or “non-targeted” recombination does not require previous knowledge of the endogenous gene sequence. The methods for expression of endogenous genes via non-homologous recombination and preparing vector constructs for non-homologous recombination are described in International Patent Applications WO 99/15650 and WO 00/49162, both of which are incorporated in their entirety by reference.

Vector constructs useful in non-homologous recombination events should contain at least a transcriptional regulatory sequence operably linked to an unpaired splice donor sequence and one or more amplifiable markers. The transcriptional regulatory sequence is typically, but not limited to, a promoter sequence. The transcriptional regulatory sequence may further comprise an enhancer sequence, in addition to the promoter sequence. The transcriptional regulatory sequence is operatively linked to a translational start codon, a signal secretion sequence and an unpaired splice donor site. The transcriptional regulatory sequence may additionally be operatively linked to a translational start codon, an epitope tag and an unpaired splice donor site; or operatively linked to a translational start codon, a signal secretion sequence, an epitope tag and an unpaired splice donor site; or operatively linked to a translational start codon, a signal secretion sequence, an epitope tag, a sequence specific protease site and an unpaired splice donor site.

Examples of amplifiable markers that may be used in the above described vectors include, but are not limited to, dihydrofolate reductase (DHFR), neomycin resistance (neo), hypoxanthine phosphoribosyl transferase (HPRT), puromycin (pac), adenosine deaminase (ada), aspartate transcarbamylase (ATC), dihydro-orotase, histidine D (his D), multidrug resistance 1 (mdr 1), xanthine-guanine phosphoribosyl transferase (gpt), glutamine synthetase (GS) and carbamyl phosphate synthase (CAD). The vector could additionally comprise a screenable marker, such as a gene encoding a cell surface protein, a fluorescent protein and/or an enzyme. A signal secretion sequence may be included on the “activation” vector construct, such that the activated gene expression product is secreted.

The regulatory sequence of the vector construct can be a constitutive promoter, an inducible promoter or a tissue specific promoter or an enhancer. The use of an inducible promoter will permit low basal levels of activated protein to be produced by the cell during routine culturing and expansion. Subsequently, the cells may then be induced to express large amounts of the desired protein during production or screening. The regulatory sequence may be isolated from cellular or viral genomes. Examples of cellular regulatory sequences include, but are not limited to, the actin gene, metallothionein I gene, collagen gene, serum albumin gene and immunoglobulin genes. Examples of viral regulatory sequences include, but are not limited to, regulatory elements from Cytomegalovirus (CMV) immediate early gene, adenovirus late genes, SV40 genes, retroviral LTRs and Herpesvirus genes (see Tables 3 and 4 for additional tissue specific and inducible regulatory sequences, respectively).

Splicing of primary transcripts, the process by which introns are removed, is directed by a splice donor site and a splice acceptor site, located at the 5′ and 3′ ends introns, respectively. The consensus sequence for splice donor sites is (A/C)AGGURAGU (where R represents a purine nucleotide), with nucleotides (A/C)AG in positions 1-3 located in the exon and nucleotides GURAGU located in the intron.

An unpaired splice donor site is defined herein as a splice donor site present on the vector construct without a downstream splice acceptor site. When the vector is integrated by non-homologous recombination into the genome of a host cell, the unpaired splice donor site becomes paired with a splice acceptor site from an endogenous gene. The splice donor site from the vector construct, in conjunction with the splice acceptor site from the endogenous gene, will then direct the excision of all of the sequences between the vector splice donor site and the endogenous splice acceptor site. Excision of these intervening sequences removes sequences that interfere with translation of the endogenous protein.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in an adenovirus vector construct of the present invention comprises a polyadenylation signal of SV40 or the protamine gene.

TABLE 3
Tissue Specific Promoters

| PROMOTER | Target |
|---|---|
| Tyrosinase | Melanocytes |
| Tyrosinase Related Protein, | Melanocytes |
| TRP-1 | |
| Prostate Specific Antigen, | Prostate Cancer |
| PSA | |
| Albumin | Liver |
| Apolipoprotein | Liver |
| Plasminogen Activator | Liver |
| Inhibitor Type-1, PAI-1 | |
| Fatty Acid Binding | Colon Epithelial Cells |
| Insulin | Pancreatic Cells |
| Muscle Creatine Kinase, | Muscle Cell |
| MCK | |
| Myelin Basic Protein, MBP | Oligodendrocytes and |
| | Glial Cells |
| Glial Fibrillary Acidic | Glial Cells |
| Protein, GFAP | |
| Neural Specific Enolase | Nerve Cells |
| Immunoglobulin Heavy | B-cells |
| Chain | |
| Immunoglobulin Light Chain | B-cells, |
| | Activated T-cells |
| T-Cell Receptor | Lymphocytes |
| HLA DQα and DQβ | Lymphocytes |
| β-Interferon | Leukocytes; |
| | Lymphocytes Fibroblasts |
| Interlukin-2 | Activated T-cells |
| Platelet Derived Growth | Erythrocytes |
| Factor | |
| E2F-1 | Proliferating Cells |
| Cyclin A | Proliferating Cells |
| α-, β-Actin | Muscle Cells |
| Haemoglobin | Erythroid Cells |
| Elastase I | Pancreatic Cells |
| Neural Cell Adhesion | Neural Cells |
| Molecule, NCAM | |

TABLE 4
Inducible Promoters

| Promoter Element | Inducer |
|---|---|
| Early Growth Response-1 | Radiation |
| Gene, egr-1 | |
| Tissue Plasmingen | Radiation |
| Activator, t-PA | |
| fos and jun | Radiation |
| Multiple Drug Resistance | Chemotherapy |
| Gene 1, mdr-1 | |
| Heat Shock Proteins; | Heat |
| hsp16, hs60, hps68, | |
| hsp70, | |
| human Plasminogen | Tumor Necrosis Factor, |
| Activator Inhibitor type-1, | TNF |
| hPAI-1 | |
| Cytochrome P-450 | Toxins |
| CYP1A1 | |
| Metal-Responsive | Heavy Metals |
| Element, MRE | |
| Mouse Mammary Tumor | Glucocorticoids |
| Virus | |
| Collagenase | Phorbol Ester |
| Stromolysin | Phorbol Ester |
| SV40 | Phorbol Ester |
| Proliferin | Phorbol Ester |
| α-2-Macroglobulin | IL-6 |
| Murine MX Gene | Interferon, Newcastle |
| | Disease Virus |
| Vimectin | Serum |
| Thyroid Stimulating | Thyroid Hormone |
| Hormone α Gene | |
| HSP70 | Ela, SV40 Large T |
| | Antigen |
| Tumor Necrosis Factor | FMA |
| Interferon | Viral Infection, dsRNA |
| Somatostatin | Cyclic AMP |
| Fibronectin | Cyclic AMP |

The cell expressing or over-expressing the gene of interest can be cultured in vitro under conditions favoring the production of the desired amounts of the expression product of the endogenous gene that has been activated or whose expression has been increased. A cell containing a vector construct which has been integrated into its genome may also be introduced into a eukaryote (e.g., a vertebrate, preferably a mammal, more preferably a human) under conditions favoring the activation or over-expression of the gene by the cell in vivo in the eukaryote. In particular embodiments, a genome-wide transcription library and protein expression library are generated (Harrington et al., 2001). Libraries are generated by random activation of gene expression (RAGE) using the above described vector constructs for non-homologous recombination.

Host cells can be derived from any eukaryotic species and can be primary, secondary, or immortalized. Furthermore, the cells can be derived from any tissue in the organism. Examples of useful tissues which cells can be isolated and activated include, but are not limited to, liver, spleen, kidney, bone marrow, thymus, heart, muscle, lung, brain, testes, ovary, islet, intestinal, skin, gall bladder, prostate, bladder and the immune hemapoietic systems.

The vector construct can be integrated into primary, secondary, or immortalized cells. Primary cells are cells that have been isolated from a vertebrate and have not been passaged. Secondary cells are primary cells that have been passaged, but are not immortalized. Immortalized cells are cell lines that can be passaged, apparently indefinitely. Examples of immortalized cell lines include, but are not limited to, HT1080, HeLa, Jurkat, 293 cells, KB carcinoma, T84 colonic epithelial cell line, Raji, Hep G2 or Hep 3B, hepatoma cell lines, A2058 melanoma, U937 lymphoma and W138 fibroblast cell.line, somatic cell hybrids and hybridomas.

Thus, to activate an endogenous gene of the present by non-homologous recombination, one would generate an “activation” vector construct comprising a regulatory sequence, one or more amplifiable markers, an epitope tag or a secretion signal sequence and an unpaired splice donor sequence. The activation construct is then introduced into a preferred eukaryotic host cell by any transfection method known in the art. Following introduction of the vector into the cell, the DNA is allowed to integrate into the host cell genome via non-homologous recombination. Integration can occur at spontaneous chromosome breaks or at artificially induced chromosomal beaks (e.g., γ irradiation, restriction enzymes). Following integration of the vector into the genome of the host cell, the genetic locus may be amplified in copy number by simultaneous or sequential selection for the one or more amplifiable markers located on the integrated vector construct. This approach facilitates the isolation of clones of cells that have amplified the locus containing the integrated vector. The cells containing the activated genes are isolated, sorted and the activated endogenous genes are isolated by PCR-based cloning (for a detailed experimental protocol, see International Application WO 99/15650, which is incorporated in its entirety by reference). One of ordinary skill in the art will appreciate, however, that any art-known method of cloning genes may be equivalently used to isolate activated genes from the sorted cells.

Transfected cells of the present invention are useful in a number of applications in humans and animals (e.g., ex vivo manipulation). In one embodiment, the cells can be implanted into a human or an animal for UP_{—}11 or OM_{—}10 polypeptide delivery in the human or animal. An UP_{—}11 or OM_{—}10 polypeptide can be delivered systemically or locally in humans for therapeutic benefits. Barrier devices, which contain transfected cells which express a therapeutic UP_{—}11 or OM_{—}10 polypeptide product and through which the therapeutic product is freely permeable, can be used to retain cells in a fixed position in vivo or to protect and isolate the cells from the host's immune system. Barrier devices are particularly useful and allow transfected immortalized cells, transfected cells from another species (transfected xenogeneic cells), or cells from a nonhistocompatibility-matched donor (transfected allogeneic cells) to be implanted for treatment of human or animal conditions. Barrier devices also allow convenient short-term (i.e., transient) therapy by providing ready access to the cells for removal when the treatment regimen is to be halted for any reason. Transfected xenogeneic and allogeneic cells may be used for short-term gene therapy, such that the gene product produced by the cells will be delivered in vivo until the cells are rejected by the host's immune system.

Transfected cells of the present invention are also useful for eliciting antibody production or for immunizing humans and animals against pathogenic agents. Implanted transfected cells can be used to deliver immunizing antigens that result in stimulation of the host's cellular and humoral immune responses. These immune responses can be designed for protection of the host from future infectious agents (i.e., for vaccination), to stimulate and augment the disease-fighting capabilities directed against an ongoing infection, or to produce antibodies directed against the antigen produced in vivo by the transfected cells that can be useful for therapeutic or diagnostic purposes. Removable barrier devices can be used to allow a simple means of terminating exposure to the antigen. Alternatively, the use of cells that will ultimately be rejected (xenogeneic or allogeneic transfected cells) can be used to limit exposure to the antigen, since antigen production will cease when the cells have been rejected.

The methods of the present invention can be used to produce primary, secondary, or immortalized cells producing UP_{—}11 or OM_{—}10 polypeptide products or anti-sense RNA. Additionally, the methods of the present invention can be used to produce cells which produce non-naturally occurring ribozymes, proteins, or nucleic acids which are useful for in vitro production of an UP_{—}11 or OM_{—}10 therapeutic product or for gene therapy.

The invention further provides a recombinant expression vector comprising a DNA molecule encoding an UP_{—}11 or OM_{—}10 polypeptide cloned into the expression vector in an antisense orientation. That is, the DNA molecule is operatively linked to a regulatory sequence in a manner which allows for expression (by transcription of the DNA molecule) of an RNA molecule which is antisense to UP_{—}11 or OM_{—}10 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, UP_{—}11 or OM_{—}10 polypeptide can be expressed in bacterial cells such as E coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation, infection or transfection techniques. As used herein, the terms ‘transformation’ and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the UP_{—}11 or OM_{—}10 polypeptide or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) UP_{—}11 or OM_{—}10 polypeptides. Accordingly, the invention further provides methods for producing UP_{—}11 or OM_{—}10 polypeptides using the host cells of the invention. In one embodiment, the method comprises culturing the host cell of invention (into which a recombinant expression vector encoding an UP_{—}11 or OM_{—}10 polypeptide has been introduced) in a suitable medium until the UP_{—}11 or OM_{—}10 polypeptide is produced. In another embodiment, the method further comprises isolating the UP_{—}11 or OM_{—}10 polypeptide from the medium or the host cell.

An expression vector comprises a polynucleotide that encodes an UP_{—}11 or OM_{—}10 polypeptide. Such a polypeptide is meant to include a sequence of nucleotide bases encoding an UP_{—}11 or OM_{—}10 polypeptide sufficient in length to distinguish said segment from a polynucleotide segment encoding a non-UP11 or OM_{—}10 polypeptide. A polypeptide of the invention can also encode biologically functional polypeptides or peptides which have variant amino acid sequences, such as with changes selected based on considerations such as the relative hydropathic score of the amino acids being exchanged. These variant sequences are those isolated from natural sources or induced in the sequences disclosed herein using a mutagenic procedure such as site-directed mutagenesis.

Preferably, the expression vectors of the present invention comprise polynucleotides that encode polypeptides comprising the amino acid residue sequence of SEQ ID NO:4, SEQ ID NO:7, SEQ ID NO:9 or SEQ ID NO:11. An expression vector can include an UP_{—}11 or OM_{—}10 polypeptide coding region itself, or any of the UP_{—}11 or OM_{—}10 polypeptides noted above or it can contain coding regions bearing selected alterations or modifications in the basic coding region of such an UP_{—}11 or OM_{—}10 polypeptide. Alternatively, such vectors or fragments can code larger polypeptides or polypeptides which nevertheless include the basic coding region. In any event, it should be appreciated that due to codon redundancy as well as biological functional equivalence, this aspect of the invention is not limited to the particular DNA molecules corresponding to the polypeptide sequences noted above.

Exemplary vectors include the mammalian expression vectors of the pCMV family including pCMV6b and pCMV6c (Chiron Corp., Emeryville Calif.). In certain cases, and specifically in the case of these individual mammalian expression vectors, the resulting constructs can require co-transfection with a vector containing a selectable marker such as pSV2neo. Via co-transfection into a dihydrofolate reductase-deficient Chinese hamster ovary cell line, such as DG44, clones expressing UP_{—}11 or OM_{—}10 polypeptides by virtue of DNA incorporated into such expression vectors can be detected.

A DNA molecule, gene or polynucleotide of the present invention can be incorporated into a vector by a number of techniques which are well known in the art. For instance, the vector pUC18 has been demonstrated to be of particular value Likewise, the related vectors M13 mp18 and M13 mp19 can be used in certain embodiments of the invention, in particular, in performing dideoxy sequencing.

An expression vector of the present invention is useful both as a means for preparing quantities of the UP_{—}11 or OM_{—}10 polypeptide-encoding DNA itself, and as a means for preparing the encoded polypeptide and peptides. It is contemplated that where UP_{—}11 or OM_{—}10 polypeptides of the invention are made by recombinant means, one can employ either prokaryotic or eukaryotic expression vectors as shuttle systems. However, in that prokaryotic systems are usually incapable of correctly processing precursor polypeptides and, in particular, such systems are incapable of correctly processing membrane associated eukaryotic polypeptides, and since eukaryotic UP_{—}11 or OM_{—}10 polypeptides are anticipated using the teaching of the disclosed invention, one likely expresses such sequences in eukaryotic hosts. However, even where the DNA segment encodes a eukaryotic UP_{—}11 or OM_{—}10 polypeptide, it is contemplated that prokaryotic expression can have some additional applicability. Therefore, the invention can be used in combination with vectors which can shuttle between the eukaryotic and prokaryotic cells. Such a system is described herein which allows the use of bacterial host cells as well as eukaryotic host cells.

Where expression of recombinant UP_{—}11 or OM_{—}10 polypeptides is desired and a eukaryotic host is contemplated, it is most desirable to employ a vector such as a plasmid, that incorporates a eukaryotic origin of replication. Additionally, for the purposes of expression in eukaryotic systems, one desires to position the UP_{—}11 or OM_{—}10 encoding sequence adjacent to and under the control of an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of or downstream with respect to the promoter chosen. Furthermore, where eukaryotic expression is anticipated, one would typically desire to incorporate into the transcriptional unit which includes the UP_{—}11 or OM_{—}10 polypeptide, an appropriate polyadenylation site.

The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these 4 plasmids in containing a translation enhancer in the sequence prior to the polylinker. While they are not directly derived from the pCMV1-5 series of vectors, the functionally similar pCMV6b and c vectors are available from the Chiron Corp. (Emeryville, Calif.) and are identical except for the orientation of the polylinker region which is reversed in one relative to the other.

The universal components of the pCMV plasmids are as follows. The vector backbone is pTZ18R (Pharmacia), and contains a bacteriophage f1 origin of replication for production of single stranded DNA and an ampicillin-resistance gene. The CMV region consists of nucleotides −760 to +3 of the powerful promoter-regulatory region of the human cytomegalovirus (Towne stain) major immediate early gene. The human growth hormone fragment (hGH) contains transcription termination and poly-adenylation signals representing sequences 1533 to 2157 of this gene. There is an Alu middle repetitive DNA sequence in this fragment. Finally, the SV40 origin of replication and early region promoter-enhancer derived from the pcD-X plasmid (HindIII to PstI fragment) described in. The promoter in this fragment is oriented such that transcription proceeds away from the CMWhGH expression cassette.

The pCMV plasmids are distinguishable from each other by differences in the polylinker region and by the presence or absence of the translation enhancer. The starting pCMV1 plasmid has been progressively modified to render an increasing number of unique restriction sites in the polylinker region. To create pCMV2, one of two EcoRI sites in pCMV1 were destroyed. To create pCMV3, pCMV1 was modified by deleting a short segment from the SV40 region (StuI to EcoRI), and in so doing made unique the PstI, SalI, and BamHI sites in the polylinker. To create pCMV4, a synthetic fragment of DNA corresponding to the 5′-untranslated region of a mRNA transcribed from the CMV promoter was added. The sequence acts as a translational enhancer by decreasing the requirements for initiation factors in polypeptide synthesis. To create pCMV5, a segment of DNA (Hpal to EcoRI) was deleted from the SV40 origin region of pCMV1 to render unique all sites in the starting polylinker.

The pCMV vectors have been successfully expressed in simian COS cells, mouse L cells, CHO cells, and HeLa cells. In several side by side comparisons they have yielded 5- to 10-fold higher expression levels in COS cells than SV40-based vectors. The pCMV vectors have been used to express the LDL receptor, nuclear factor 1, GS alpha polypeptide, polypeptide phosphatase, synaptophysin, synapsin, insulin receptor, influenza hemmagglutinin, androgen receptor, sterol 26-hydroxylase, steroid 17- and 21-hydroxylase, cytochrome P-450 oxidoreductase, beta-adrenergic receptor, folate receptor, cholesterol side chain cleavage enzyme, and a host of other cDNAs. It should be noted that the SV40 promoter in these plasmids can be used to express other genes such as dominant selectable markers. Finally, there is an ATG sequence in the polylinker between the HindIII and PstI sites in pCMU that can cause spurious translation initiation. This codon should be avoided if possible in expression plasmids.

In yet another embodiment, the present invention provides recombinant host cells transformed, infected or transfected with polynucleotides that encode UP_{—}11 or OM_{—}10 polypeptides, as well as transgenic cells derived from those transformed or transfected cells. Preferably, the recombinant host cells of the present invention are transfected with a polynucleotide of SEQ ID NO:1, SEQ ID NO:2, SEQ ID NO:3 SEQ ID NO:5, SEQ ID NO:6, SEQ ID NO:8 or SEQ ID NO:10. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection (Sambrook et al., 1989).

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

The use of adenovirus as a vector for cell transfection is well known in the art. Adenovirus vector-mediated cell transfection has been reported for various cells.

A transfected cell can be prokaryotic or eukaryotic. Preferably, the host cells of the invention are eukaryotic host cells. The recombinant host cells of the invention may be COS-1 cells. Where it is of interest to produce a human UP_{—}11 or OM_{—}10 polypeptide, cultured mammalian or human cells are of particular interest.

In another aspect, the recombinant host cells of the present invention are prokaryotic host cells. Preferably, the recombinant host cells of the invention are bacterial cells of the DH5 α strain of Escherichia coli. In general, prokaryotes are preferred for the initial cloning of DNA sequences and constructing the vectors useful in the invention. For example, E coli K12 strains can be particularly useful. Other microbial strains which can be used include E. coli B, and E. coliₓ976 (ATCC No. 31537). These examples are, of course, intended to be illustrative rather than limiting.

Prokaryotes can also be used for expression. The aforementioned strains, as well as E. coli W31 10 (ATCC No. 273325), bacilli such as Bacillus subtilis, or other enterobacteriaceae such as Salmonella typhimurium or Serratia marcesans, and various Pseudomonas species can be used.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli can be transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the β-lactamase (penicillinase) and lactose promoter systems and a tryptophan (TRP) promoter system (European Application No. EP 0036776). While these are the most commonly used, other microbial promoters have been discovered and utilized, and details concerning their nucleotide sequences have been published, enabling a skilled worker to introduce functional promoters into plasmid vectors.

In addition to prokaryotes, eukaryotic microbes such as yeast can also be used. Saccharomyces cerevisiase or common baker's yeast is the most commonly used among eukaryotic microorganisms, although a number of other strains are commonly available. For expression in Saccharomyces, the plasmid YRp7, for example, is commonly used. This plasmid already contains the trpl gene which provides a selection marker for a mutant strain of yeast lacking the ability to grow in tryptophan, for example ATCC No. 44076 or PEP4-1. The presence of the trpl lesion as a characteristic of the yeast host cell genome then provides an effective environment for detecting transformation by growth in the absence of tryptophan.

Suitable promoter sequences in yeast vectors include the promoters for 3-phosphoglycerate kinase or other glycolytic enzymes such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also introduced into the expression vector downstream from the sequences to be expressed to provide polyadenylation of the mRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin or replication and termination sequences is suitable.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO and HeLa cells, Chinese hamster ovary (CHO) cell lines, and W138, BHK, COSM6, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

For use in mammalian cells, the control functions on the expression vectors are often derived from viral material. For example, commonly used promoters are derived from polyoma, Adenovirus 2, Cytomegalovirus and most frequently Simian Virus 40 (SV40). The early and late promoters of SV40 virus are particularly useful because both are obtained easily from the virus as a fragment which also contains the SV40 viral origin of rep. Smaller or larger SV40 fragments can also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication can be provided with by construction of the vector to include an exogenous origin, such as can be derived from SV40 or other viral (e.g., Polyoma, Adeno, VSV, BPV, CMV) source, or can be provided by the host cell chromosomal replication mechanism. If the vector is integrated into the host cell chromosome, the latter is often sufficient.

In yet another embodiment, the present invention contemplates a process or method of preparing UP_{—}11 or OM_{—}10 polypeptides comprising transfecting cells with polynucleotide that encode UP_{—}11 or OM_{—}10 polypeptides to produce transformed host cells; and maintaining the transformed host cells under biological conditions sufficient for expression of the polypeptide. Preferably, the transformed host cells are eukaryotic cells. Alternatively, the host cells are prokaryotic cells. More preferably, the prokaryotic cells are bacterial cells of the DH5-α strain of Escherichia coli. Even more preferably, the polynucleotide transfected into the transformed cells comprise the nucleic acid sequence of SEQ ID NO:1, SEQ ID NO:2, SEQ ID NO:3 SEQ ID NO:5, SEQ ID NO:6, SEQ ID NO:8 or SEQ ID NO:10. Additionally, transfection is accomplished using an expression vector disclosed above.

A host cell used in the process is capable of expressing a functional, recombinant UP_{—}11 or OM_{—}10 polypeptide. A preferred host cell is a Chinese hamster ovary cell. However, a variety of cells are amenable to a process of the invention, for instance, yeast cells, human cell lines, and other eukaryotic cell lines known well to those of skill in the art.

Following transfection, the cell is maintained under culture conditions for a period of time sufficient for expression of an UP_{—}11 or OM_{—}10 receptor polypeptide. Culture conditions are well known in the art and-include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C., more preferably from about 30° C. to about 40° C. and, even more preferably about 37° C. pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Other biological conditions needed for transfection and expression of an encoded polypeptide are well known in the art.

Transfected cells are maintained for a period of time sufficient for expression of an UP_{—}11 or OM_{—}10 polypeptide. A suitable time depends inter alia upon the cell type used and is readily determinable by a skilled artisan. Typically, maintenance time is from about 2 to about 14 days.

Recombinant UP_{—}11 or OM_{—}10 polypeptide is recovered or collected either from the transfected cells or the medium in which those cells are cultured. Recovery comprises isolating and purifying the UP 11 or OM_{—}10 polypeptide. Isolation and purification techniques for polypeptides are well known in the art and include such procedures as precipitation, filtration, chromatography, electrophoresis and the like.

E. Transgenic Animals

In certain preferred embodiments, the invention pertains to nonhuman animals with somatic and germ cells having a functional disruption of at least one, and more preferably both, alleles of an endogenous G-polypeptide coupled receptor (GPCR) gene of the present invention. Adcordingly, the invention provides viable animals having a mutated UP_{—}11 or OM_{—}10 gene, and thus lacking UP_{—}11 or OM_{—}10 activity. These animals will produce substantially reduced amounts of an UP_{—}11 or OM_{—}10 in response to stimuli that produce normal amounts of an UP_{—}11 or OM_{—}10 in wild type control animals. The animals of the invention are useful, for example, as standard controls by which to evaluate UP_{—}11 or OM_{—}10 inhibitors, as recipients of a normal human UP_{—}11 or OM_{—}10 gene to thereby create a model system for screening human UP_{—}11 or OM_{—}10 inhibitors in vivo, and to identify disease states for treatment with UP_{—}11 or OM_{—}10 inhibitors. The animals are also useful as controls for studying the effect of ligands on the UP_{—}11 or OM_{—}10 polypeptides.

In the transgenic nonhuman animal of the invention, the UP_{—}11 or OM_{—}10 gene preferably is disrupted by homologous recombination between the endogenous allele and a mutant UP_{—}11 or OM_{—}10 polynucleotide, or portion thereof, that has been introduced into an embryonic stem cell precursor of the animal. The embryonic stem cell precursor is then allowed to develop, resulting in an animal having a functionally disrupted UP_{—}11 or OM_{—}10 gene. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. The animal may have one UP_{—}11 or OM_{—}10 gene allele functionally disrupted (i.e., the animal may be heterozygous for the mutation), or more preferably, the animal has both UP_{—}11 or OM_{—}10 gene alleles functionally disrupted (i.e., the animal can be homozygous for the mutation).

In one embodiment of the invention, functional disruption of both UP_{—}11 or OM_{—}10 gene alleles produces animals in which expression of the UP_{—}11 or OM_{—}10 gene product in cells of the animal is substantially absent relative to non-mutant animals. In another embodiment, the UP_{—}11 or OM 10 gene alleles can be disrupted such that an altered (i.e., mutant) UP_{—}11 or OM_{—}10 gene product is produced in cells of the animal. A preferred nonhuman animal of the invention having a functionally disrupted UP_{—}11 or OM_{—}10 gene is a mouse. Given the essentially complete inactivation of UP_{—}11 or OM_{—}10 function in the homozygous animals of the invention and the about 50% inhibition of UP_{—}11 or OM_{—}10 function in the heterozygous animals of the invention, these animals are useful as positive controls against which to evaluate the effectiveness of UP_{—}11 or OM_{—}10 inhibitors. For example, a stimulus that normally induces production or activity of UP_{—}11 or OM_{—}10 can be administered to a wild type animal (i.e., an animal having a non-mutant UP_{—}11 or OM_{—}10 gene) in the presence of an UP_{—}11 or OM_{—}10 inhibitor to be tested and production or activity of UP_{—}11 or OM_{—}10 by the animal can be measured. The UP_{—}11 or OM_{—}10 response in the wild type animal can then be compared to the UP_{—}11 or OM_{—}10 response in the heterozygous and homozygous animals of the invention, similarly administered the UP_{—}11 or OM_{—}10 stimulus, to determine the percent of maximal UP_{—}11 or OM_{—}10 inhibition of the test inhibitor.

Additionally, the animals of the invention are useful for determining whether a particular disease condition involves the action of UP_{—}11 or OM_{—}10 and thus can be treated by an UP_{—}11 or OM_{—}10 inhibitor. For example, an attempt can be made to induce a disease condition in an animal of the invention having a functionally disrupted UP_{—}11 or OM_{—}10 gene. Subsequently, the susceptibility or resistance of the animal to the disease condition can be determined. A disease condition that is treatable with an UP_{—}11 or OM_{—}10 inhibitor can be identified based upon resistance of an animal of the invention to the disease condition. Another aspect of the invention pertains to a transgenic nonhuman animal having a functionally disrupted endogenous UP_{—}11 or OM_{—}10 gene but which also carries in its genome, and expresses, a transgene encoding a heterologous UP_{—}11 or OM_{—}10 (i.e., a GPCR from another species). Preferably, the animal is a mouse and the heterologous UP_{—}11 or OM_{—}10 is a human UP_{—}11 or OM_{—}10 (e.g., SEQ ID NO:1, SEQ ID NO:2, SEQ ID NO:3 and SEQ ID NO:8). An animal of the invention which has been reconstituted with human UP_{—}11 or OM_{—}10 can be used to identify agents that inhibit human UP_{—}11 or OM_{—}10 in vivo. For example, a stimulus that induces production and/or activity of UP_{—}11 or OM_{—}10 can be administered to the animal in the presence and absence of an agent to be tested and the UP_{—}11 or OM_{—}10 response in the animal can be measured. An agent that inhibits human UP_{—}11 or OM_{—}10 in vivo can be identified based upon a decreased UP_{—}11 or OM_{—}10 response in the presence of the agent compared to the UP_{—}11 or OM_{—}10 response in the absence of the agent. As used herein, a “transgene” is exogenous DNA which is integrated into the genome of a cell from which a transgenic animal develops and which remains in the genome of the mature animal, thereby directing the expression of an encoded gene product in one or more cell types or tissues of the transgenic animal.

Yet another aspect of the invention pertains to a polynucleotide construct for functionally disrupting an UP_{—}11 or OM_{—}10 gene in a host cell. The nucleic acid construct comprises: a) a nonhomologous replacement portion; b) a first homology region located upstream of the nonhomologous replacement portion, the first homology region having a nucleotide sequence with substantial identity to a first UP_{—}11 or OM 10 gene sequence; and c) a second homology region located downstream of the nonhomologous replacement portion, the second homology region having a nucleotide sequence with substantial identity to a second UP_{—}11 or OM_{—}10 gene sequence, the second UP_{—}11 or OM_{—}10 gene sequence having a location downstream of the first UP_{—}11 or OM_{—}10 gene sequence in a naturally occurring endogenous UP_{—}11 or OM_{—}10 gene. Additionally, the first and second homology regions are of sufficient length for homologous recombination between the nucleic acid construct and an endogenous UP_{—}11 or OM_{—}10 gene in a host cell when the nucleic acid molecule is introduced into the host cell. As used herein, a “homologous recombinant animal” is a non-human animal, preferably a mammal, more preferably a mouse, in which an endogenous UP_{—}11 or OM_{—}10 gene has been altered by homologous recombination between the endogenous gene and an exogenous DNA molecule introduced into a cell of the animal, e.g., an embryonic cell of the animal, prior to development of the animal.

In a preferred embodiment, the nonhomologous replacement portion comprises a positive selection expression cassette, preferably including a neomycin phosphotransferase gene operatively linked to a regulatory element(s). In another preferred embodiment, the nucleic acid construct also includes a negative selection expression cassette distal to either the upstream or downstream homology regions. A preferred negative selection cassette includes a herpes simplex virus thymidine kinase gene operatively linked to a regulatory element(s). Another aspect of the invention pertains to recombinant vectors into which the nucleic acid construct of the invention has been incorporated.

Yet another aspect of the invention pertains to host cells into which the nucleic acid construct of the invention has been introduced to thereby allow homologous recombination between the nucleic acid construct and an endogenous UP_{—}11 or OM_{—}10 gene of the host cell, resulting in functional disruption of the endogenous UP_{—}11 or OM_{—}10 gene. The host cell can be a mammalian cell that normally expresses UP_{—}11 or OM_{—}10, such as a human neuron, or a pluripotent cell, such as a mouse embryonic stem cell. Further development of an embryonic stem cell into which the nucleic acid construct has been introduced and homologously recombined with the endogenous UP_{—}11 or OM_{—}10 gene produces a transgenic nonhuman animal having cells that are descendant from the embryonic stem cell and thus carry the UP_{—}11 or OM_{—}10 gene disruption in their genome. Animals that carry the UP_{—}11 or OM_{—}10 gene disruption in their germine can then be selected and bred to produce animals having the UP_{—}11 or OM_{—}10 gene disruption in all somatic and germ cells. Such mice can then be bred to homozygosity for the UP_{—}11 or OM_{—}10 gene disruption.

It is contemplated that in some instances the genome of a transgenic animal of the present invention will have been altered through the stable introduction of one or more of the UP_{—}11 or OM_{—}10 polynucleotide compositions described herein, either native, synthetically modified or mutated. As described herein, a “transgenic animal” refers to any animal, preferably a non-human mammal (e.g. mouse, rat, rabbit, squirrel, hamster, rabbits, guinea pigs, pigs, micro-pigs, prairie, baboons, squirrel monkeys and chimpanzees, etc), bird or an amphibian, in which one or more cells contain heterologous nucleic acid introduced by way of human intervention, such as by transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

A transgenic animal of the invention can be created by introducing an UP_{—}11 or OM_{—}10 polypeptide encoding nucleic acid into the male pronuclei of a fertilized oocyte, e.g., by microinjection, retroviral infection, and allowing the oocyte to develop in a pseudopregnant female foster animal. The human UP_{—}11 or OM_{—}10 polynucleotide sequence of SEQ ID NO:1, SEQ ID NO:2, SEQ ID NO:3, or SEQ ID NO:8 can be introduced as a transgene into the genome of a non-human animal.

Moreover, a non-human homologue of the human UP_{—}11 or OM_{—}10 gene, such as a mouse UP_{—}11 or OM_{—}10 gene, can be isolated based on hybridization to the human UP_{—}11 or OM_{—}10 polynucleotide (described above) and used as a transgene. Intronic sequences and polyadenylation signals can also be included in the transgene to increase the efficiency of expression of the transgene. A tissue-specific regulatory sequence(s) can be operably linked to the UP_{—}11 or OM_{—}10 transgene to direct expression of an UP_{—}11 or OM_{—}10 polypeptide to particular cells. Methods for generating transgenic animals via embryo manipulation and microinjection, particularly animals such as mice, have become conventional in the art and are described, for example, in U.S. Pat. Nos. 4,736,866, 4,870,009 and 4,873,191; and in Hogan, 1986. Similar methods are used for production of other transgenic animals. A transgenic founder animal can be identified based upon the presence of the UP_{—}11 or OM_{—}10 transgene in its genome and/or expression of UP_{—}11 or OM_{—}10 mRNA in tissues or cells of the animals. A transgenic founder animal can then be used to breed additional animals carrying the transgene. Moreover, transgenic animals carrying a transgene encoding an UP_{—}11 or OM_{—}10 polypeptide can further be bred to other transgenic animals carrying other transgenes.

To create a homologous recombinant animal, a vector is prepared which contains at least a fragment of an UP_{—}11 or OM_{—}10 gene into which a deletion, addition or substitution has been introduced to thereby alter, e.g., functionally disrupt, the UP_{—}11 or OM_{—}10 gene. The UP_{—}11 or OM_{—}10 gene can be a human gene (e.g., from a human genomic clone isolated from a human genomic library such as SEQ ID NO:1, SEQ ID NO:2, SEQ ID NO:3 or SEQ ID NO:8), but more preferably is a non-human homologue of a human GPCR gene (e.g., the murine polynucleotide of SEQ ID NO:5, SEQ ID NO:6 or SEQ ID NO:10). The mouse UP_{—}11 or OM_{—}10 gene can be used to construct a homologous recombination vector suitable for altering an endogenous UP_{—}11 or OM_{—}10 gene in the mouse genome. In a preferred embodiment, the vector is designed such that, upon homologous recombination, the endogenous UP_{—}11 or OM_{—}10 gene is functionally disrupted (i.e., no longer encodes a functional protein; also referred to as a “knock out” vector.

Alternatively, the vector can be designed such that, upon homologous recombination, the endogenous UP_{—}11 or OM_{—}10 gene is mutated or otherwise altered but still encodes functional protein (e.g., the upstream regulatory region can be altered to thereby alter the expression of the endogenous UP_{—}11 or OM_{—}10 polypeptide). In the homologous recombination vector, the altered fragment of the UP_{—}11 or OM_{—}10 gene is flanked at its 5′ and 3′ ends by additional nucleic acid of the UP_{—}11 or OM_{—}10 gene (e.g., flanking, noncoding sequences of SEQ ID NO:1 are 5′ nucleotides 1-297 and 3′ nucleotides 1,654-3,824, noncoding sequences of SEQ ID NO:2 are 3′ nucleotides 1,314-3,546, noncoding sequences of SEQ ID NO:3 are 5′ nucleotides 1-670 and 3′ nucleotides 2,027-3,779) to allow for homologous recombination to occur between the exogenous UP_{—}11 or OM_{—}10 gene carried by the vector and an endogenous UP_{—}11 or OM_{—}10 gene in an embryonic stem cell. The additional flanking UP_{—}11 or OM_{—}10 nucleic acid is of sufficient length for successful homologous recombination with the endogenous gene.

Typically, several kilobases of flanking DNA (both at the 5′ and 3′ ends) are included in the vector (see e.g., Thomas and Capecchi, 1987, for a description of homologous recombination vectors). The vector is introduced into an embryonic stem cell line (e.g., by electroporation) and cells in which the introduced UP_{—}11 or OM_{—}10 gene has homologously recombined with the endogenous UP_{—}11 or OM_{—}10 gene are selected (see e.g., Li et al., 1992). The selected cells are then injected into a blastocyst of an animal (e.g., a mouse) to form aggregation chimeras (see e.g., Bradley, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, 1991; and in International Application Nos. WO 90/11354; WO 91/01140; WO 92/0968; and WO 93/04169.

In another embodiment, transgenic non-human animals can be produced which contain selected systems which allow for regulated expression of the transgene. One example of such a system is the cre/loxP recombinase system of bacteriophage PL. For a description of the cre/loxP recombinase system, see, e.g., Lakso et al., 1992. Another example of a recombinase system is the FLP recombinase system of Saccharomyces cerevisiae (O'Gonnan et al., 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al, 1997, and International Application Nos. WO 97/07668 and WO 97/07669. In brief, a cell, e.g., a somatic cell, from/the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

F. Uses and Methods of the Invention

The nucleic acid molecules, polypeptides, polypeptide homologues, modulators, and antibodies described herein can be used in, but are limited to, one or more of the following methods: a) drug screening assays; b) diagnostic assays particularly in disease identification, allelic screening and pharmocogenetic testing; c) methods of treatment; d) pharmacogenomics; and e) monitoring of effects during clinical trials. An UP_{—}11 or OM_{—}10 polypeptide of the invention can be used as a drug target for developing agents to modulate the activity of the UP_{—}11 or OM_{—}10 polypeptide. The isolated nucleic acid molecules of the invention can be used to express UP_{—}11 or OM_{—}10 polypeptide (e.g., via a recombinant expression vector in a host cell or in gene therapy applications), to detect UP_{—}11 or OM_{—}10 mRNA (e.g., in a biological sample) or a naturally occurring or recombinantly generated genetic mutation in an UP_{—}11 or OM_{—}10 gene, and to modulate UP_{—}11 or OM_{—}10 polypeptide activity, as described further below. In addition, the UP_{—}11 or OM_{—}10 polypeptides can be used to screen drugs or compounds which modulate UP_{—}11 or OM_{—}10 polypeptide activity. Moreover, the anti-UP_{—}11 or OM_{—}10 antibodies of the invention can be used to detect and isolate an UP_{—}11 or OM_{—}10 polypeptide, particularly fragments of an UP_{—}11 or OM_{—}10 polypeptide present in a biological sample, and to modulate UP_{—}11 or OM_{—}10 polypeptide activity.

Drug Screening Assays

The invention provides methods for identifying compounds or agents that can be used to treat disorders characterized by (or associated with) aberrant or abnormal UP_{—}11 or OM_{—}10 nucleic acid expression and/or UP_{—}11 or OM_{—}10 polypeptide activity. These methods are also referred to herein as drug screening assays and typically include the step of screening a candidate/test compound or agent to identify compounds that are an agonist or antagonist of an UP_{—}11 or OM_{—}10 polypeptide, and specifically for the ability to interact with (e.g., bind to) an UP_{—}11 or OM_{—}10 polypeptide, to modulate the interaction of an UP_{—}11 or OM_{—}10 polypeptide and a target molecule, and/or to modulate UP_{—}11 or OM_{—}10 nucleic acid expression and/or UP_{—}11 or OM_{—}10 polypeptide activity.

Candidate/test compounds or agents which have one or more of these abilities can be used as drugs to treat disorders characterized by aberrant or abnormal UP_{—}11 or OM_{—}10 nucleic acid expression and/or UP_{—}11 or OM_{—}10 polypeptide activity. Candidate/test compounds include, for example, 1) peptides such as soluble peptides, including Ig-tailed fusion peptides and members of random peptide libraries and combinatorial chemistry-derived molecular libraries made of D- and/or L-configuration amino acids; 2) phosphopeptides (e.g., members of random and partially degenerate, directed phosphopeptide libraries, see, e.g., Songyang et al., 1993); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

In one embodiment, the invention provides assays for screening candidate/test compounds which interact with (e.g., bind to) an UP_{—}11 or OM_{—}10 polypeptide. Typically, the assays are recombinant cell based or cell-free assays which include the steps of combining a cell expressing an UP_{—}11 or OM_{—}10 polypeptide or a bioactive fragment thereof, or an isolated UP_{—}11 or OM_{—}10 polypeptide, and a candidate/test compound, e.g., under conditions which allow for interaction of (e.g., binding of) the candidate/test compound to the UP_{—}11 or OM_{—}10 polypeptide or fragment thereof to form a complex, and detecting the formation of a complex, in which the ability of the candidate compound to interact with (e.g., bind to) the UP_{—}11 or OM_{—}10 polypeptide or fragment thereof is indicated by the presence of the candidate compound in the complex. Formation of complexes between the UP_{—}11 or OM_{—}10 polypeptide and the candidate compound can be detected using competition binding assays, and can be quantitated, for example, using standard immunoassays.

In another embodiment, the invention provides screening assays to identify candidate/test compounds which modulate (e.g., stimulate or inhibit) the interaction (and most likely UP_{—}11 or OM_{—}10 polypeptide activity as well) between an UP_{—}11 or OM_{—}10 polypeptide and a molecule (target molecule) with which the UP_{—}11 or OM_{—}10 polypeptide normally interacts. Examples of such target molecules include proteins in the same signaling path as the UP_{—}11 or OM_{—}10 polypeptide, e.g., proteins which may function upstream (including both stimulators and inhibitors of activity) or downstream of the UP_{—}11 or OM_{—}10 polypeptide in, for example, a cognitive function signaling pathway or in a pathway involving UP_{—}11 or OM_{—}10 polypeptide activity, e.g., a G protein or other interactor involved in cAMP or phosphatidylinositol turnover, and/or adenylate cyclase or phospholipase C activation. Typically, the assays are recombinant cell based assays which include the steps of combining a cell expressing an UP_{—}11 or OM_{—}10 polypeptide, or a bioactive fragment thereof, an UP_{—}11 or OM_{—}10 polypeptide target molecule (e.g., an UP_{—}11 or OM_{—}10 ligand) and a candidate/test compound, e.g., under conditions wherein but for the presence of the candidate compound, the UP_{—}11 or OM_{—}10 polypeptide or biologically active fragment thereof interacts with (e.g., binds to) the target molecule, and detecting the formation of a complex which includes the UP_{—}11 or OM_{—}10 polypeptide and the target molecule or detecting the interaction/reaction of the UP_{—}11 or OM_{—}10 polypeptide and the target molecule.

Detection of complex formation can include direct quantitation of the complex by, for example, measuring inductive effects of the UP_{—}11 or OM_{—}10 polypeptide. A statistically significant change, such as a decrease, in the interaction of the UP_{—}11 or OM_{—}10 polypeptide and target molecule (e.g., in the formation of a complex between the UP_{—}11 or OM_{—}10 polypeptide and the target molecule) in the presence of a candidate compound (relative to what is detected in the absence of the candidate compound) is indicative of a modulation (e.g., stimulation or inhibition) of the interaction between the UP_{—}11 or OM_{—}10 polypeptide and the target molecule. Modulation of the formation of complexes between the UP_{—}11 or OM_{—}10 polypeptide and the target molecule can be quantitated using, for example, an immunoassay.

To perform cell free drug screening assays, it is desirable to immobilize either the UP_{—}11 or OM_{—}10 polypeptide or its target molecule to facilitate separation of complexes from uncomplexed forms of one or both of the proteins, as well as to accommodate automation of the assay. Interaction (e.g., binding of) of the UP_{—}11 or OM_{—}10 polypeptide to a target molecule, in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows the protein to be bound to a matrix. For example, glutathione-S-transferase/UP_{—}11 or OM_{—}10 fusion proteins can be adsorbed onto glutathione sepharose beads (Sigma Chemical, St. Louis, Mo.) or glutathione derivatized microtitre plates, which are then combined with the cell lysates (e.g., ³⁵S labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of UP_{—}11 or OM_{—}10-binding protein found in the bead fraction quantitated from the gel using standard electrophoretic techniques.

Other techniques for immobilizing proteins on matrices can also be used in the drug screening assays of the invention. For example, either the UP_{—}11 or OM_{—}10 polypeptide or its target molecule can be immobilized utilizing conjugation of biotin and streptavidin. Biotinylated UP_{—}11 or OM_{—}10 polypeptide molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with an UP_{—}11 or OM_{—}10 polypeptide but which do not interfere with binding of the protein to its target molecule can be derivatized to the wells of the plate, and UP_{—}11 or OM_{—}10 polypeptide trapped in the wells by antibody conjugation. As described above, preparations of an UP_{—}11 or OM_{—}10-binding protein and a candidate compound are incubated in the UP_{—}11 or OM_{—}10 polypeptide-presenting wells of the plate, and the amount of complex trapped in the well can be quantitated. Methods for detecting such complexes, in addition to those described above for the GST-immobilized complexes, include immunodetection of complexes using antibodies reactive with the UP_{—}11 or OM_{—}10 polypeptide target molecule, or which are reactive with UP_{—}11 or OM_{—}10 polypeptide and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant or abnormal UP_{—}11 or OM_{—}10 nucleic acid expression or UP_{—}11 or OM_{—}10 polypeptide activity. This method typically includes the step of assaying the ability of the compound or agent to modulate the expression of the UP_{—}11 or OM_{—}10 nucleic acid or the activity of the UP_{—}11 or OM_{—}10 polypeptide thereby identifying a compound for treating a disorder characterized by aberrant or abnormal UP_{—}11 or OM_{—}10 nucleic acid expression or UP_{—}11 or OM_{—}10 polypeptide activity. Methods for assaying the ability of the compound or agent to modulate the expression of the UP_{—}11 or OM_{—}10 nucleic acid or activity of the UP_{—}11 or OM_{—}10 polypeptide are typically cell-based assays. For example, cells which are sensitive to ligands which transduce signals via a pathway involving an UP_{—}11 or OM_{—}10 polypeptide can be induced to overexpress an UP_{—}11 or OM_{—}10 polypeptide in the presence and absence of a candidate compound.

Candidate compounds which produce a statistically significant change in UP_{—}11 or OM_{—}10 polypeptide-dependent responses (either stimulation or inhibition) can be identified. In one embodiment, expression of the UP_{—}11 or OM_{—}10 nucleic acid or activity of an UP_{—}11 or OM_{—}10 polypeptide is modulated in cells and the effects of candidate compounds on the readout of interest (such as cAMP or phosphatidylinositol turnover) are measured. For example, the expression of genes which are up- or down-regulated in response to an UP_{—}11 or OM_{—}10 polypeptide-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of an UP_{—}11 or OM_{—}10 polypeptide or UP_{—}11 or OM_{—}10 polypeptide target molecules can also be measured, for example, by immunoblotting.

Alternatively, modulators of UP_{—}11 or OM_{—}10 gene expression (e.g., compounds which can be used to treat a disorder characterized by aberrant or abnormal UP_{—}11 or OM_{—}10 nucleic acid expression or UP_{—}11 or OM_{—}10 polypeptide activity) can be identified in a method wherein a cell is contacted with a candidate compound and the expression of UP_{—}11 or OM_{—}10 mRNA or protein in the cell is determined. The level of expression of UP_{—}11 or OM_{—}10 mRNA or protein in the presence of the candidate compound is compared to the level of expression of UP_{—}11 or OM_{—}10 mRNA or protein in the absence of the candidate compound. The candidate compound can then be identified as a modulator of UP_{—}11 or OM_{—}10 nucleic acid expression based on this comparison and be used to treat a disorder characterized by aberrant UP_{—}11 or OM_{—}10 nucleic acid expression. For example, when expression of UP_{—}11 or OM_{—}10 mRNA or protein is greater (statistically significantly greater) in the presence of the candidate compound than in its absence, the candidate compound is identified as a stimulator of UP_{—}11 or OM_{—}10 nucleic acid expression. Alternatively, when UP_{—}11 or OM_{—}10 nucleic acid expression is less (statistically significantly less) in the presence of the candidate compound than in its absence, the candidate compound is identified as an inhibitor of UP_{—}11 or OM_{—}10 nucleic acid expression. The level of UP_{—}11 or OM_{—}10 nucleic acid expression in the cells can be determined by methods described herein for detecting UP_{—}11 or OM_{—}10 mRNA or protein.

In certain aspects of the invention, UP_{—}11 or OM_{—}10 polypeptides or portions thereof can be used as “bait proteins” in a two-hybrid assay or three-hybrid assay (see, e.g., U.S. Pat. No. 5,283,317; U.S. Statutory Invention Registration No. H1,892; Zervos et al., 1993; Madura et al., 1993; Bartel et al., 1993(a); Iwabuchi et al., 1993; International Application No. WO 94/10300), to identify other proteins, which bind to or interact with UP_{—}11 or OM_{—}10 (“UP_{—}11 or OM_{—}10-binding proteins” or “UP_{—}11- or OM_{—}10-bp”) and are involved in UP_{—}11 or OM_{—}10 activity. Such UP_{—}11 or OM_{—}10-binding proteins are also likely to be involved in the propagation of signals by the UP_{—}11 or OM_{—}10 polypeptides or UP_{—}11 or OM_{—}10 targets as, for example, downstream elements of an UP_{—}11 or OM_{—}10-mediated signaling pathway. Alternatively, such UP_{—}11 or OM_{—}10-binding proteins may be UP_{—}11 or OM_{—}10 inhibitors.

Thus, in certain embodiments, the invention contemplates determining protein:protein interactions, e.g., UP_{—}11 or OM_{—}10 and an UP_{—}11 or OM_{—}10 binding protein. The yeast two-hybrid system is extremely useful for studying protein:protein interactions. Variations of the system are available for screening yeast phagemid (Harper et al., 1993; Elledge et al., 1991) or plasmid (Bartel et al., 1993(a),(b); Finley and Brent, 1994) cDNA libraries to clone interacting proteins, as well as for studying known protein pairs. Recently, a two-hybrid method for high volume screening for specific inhibitors of protein:protein interactions and a two-hybrid screen that identifies many different interactions between protein pairs at once have been described (see, U.S. Statutory Invention Registration No. H1,892).

The success of the two-hybrid system relies upon the fact that the DNA binding and polymerase activation domains of many transcription factors, such as GAL4, can be separated and then rejoined to restore functionality (Morin et al., 1993). Briefly, the assay utilizes two different DNA constructs. In one construct, the gene that codes for an UP_{—}11 or OM_{—}10 polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an UP_{—}11- or OM_{—}10-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the UP_{—}11 or OM_{—}10 polypeptide.

Modulators of UP_{—}11 or OM_{—}10 polypeptide activity and/or UP_{—}11 or OM_{—}10 nucleic acid expression identified according to these drug screening assays can be used to treat, for example, nervous system disorders. These methods of treatment include the steps of administering the modulators of UP_{—}11 or OM_{—}10 polypeptide activity and/or nucleic acid expression, e.g., in a pharmaceutical composition as described herein, to a subject in need of such treatment, e.g., a subject with a disorder described herein.

Diagnostic Assays

The invention further provides a method for detecting the presence of an UP_{—}11 or OM_{—}10 polypeptide or UP_{—}11 or OM_{—}10 nucleic acid molecule, or fragment thereof, in a biological sample. The method involves contacting the biological sample with a compound or an agent capable of detecting UP_{—}11 or OM_{—}10 polypeptide or mRNA such that the presence of UP_{—}11 or OM_{—}10 polypeptide/encoding nucleic acid molecule is detected in the biological sample. A preferred agent for detecting UP_{—}11 or OM_{—}10 mRNA is a labeled or labelable nucleic acid probe capable of hybridizing to UP_{—}11 or OM_{—}10 mRNA. The nucleic acid probe can be, for example, the full-length UP_{—}11 or OM_{—}10 cDNA of SEQ ID NO: 1, SEQ ID NO: 3, SEQ ID NO: 5, SEQ ID NO: 6, SEQ ID NO: 7, SEQ ID NO: 8 or SEQ ID NO: 10, or a fragment thereof, such as an oligonucleotide of at least 15, 30, 50, 100, 250 or 500 nucleotides in length and sufficient to specifically hybridize under stringent conditions to UP_{—}11 or OM_{—}10 mRNA. A preferred agent for detecting UP_{—}11 or OM_{—}10 polypeptide is a labeled or labelable antibody capable of binding to UP_{—}11 OR OM_{—}10 polypeptide. Antibodies can be polyclonal, or more preferably, monoclonal. An intact antibody, or a fragment thereof (e.g., Fab or F(ab′)₂) can be used. The term “labeled or labelable,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect UP_{—}11 or OM_{—}10 mRNA or protein in a biological sample in vitro as well as in vivo. For example, in vitro techniques for detection of UP_{—}11 or OM_{—}10 mRNA include Northern hybridizations and in situ hybridizations. In vitro techniques for detection of UP_{—}11 or OM_{—}10 polypeptide include enzyme linked immunosorbent assays (ELISAs), Western blots, immunoprecipitations and immunofluorescence. Alternatively, UP_{—}11 or OM_{—}10 polypeptide can be detected in vivo in a subject by introducing into the subject a labeled anti-UP_{—}11 or OM_{—}10 antibody. For example, the antibody can be labeled with a radioactive marker whose presence and location in a subject can be detected by standard imaging techniques. Particularly useful are methods which detect the allelic variant of an UP_{—}11 or OM_{—}10 polypeptide expressed in a subject and methods which detect fragments of an UP_{—}11 or OM_{—}10 polypeptide in a sample.

The invention also encompasses kits for detecting the presence of an UP_{—}11 or OM_{—}10 polypeptide in a biological sample. For example, the kit can comprise reagents such as a labeled or labelable compound or agent capable of detecting UP_{—}11 or OM_{—}10 polypeptide or mRNA in a biological sample; means for determining the amount of UP_{—}11 or OM 10 polypeptide in the sample; and means for comparing the amount of UP_{—}11 or O_{—}10 polypeptide in the sample with a standard. The compound or agent can be packaged in a suitable container. The kit can further comprise instructions for using the kit to detect UP_{—}11 or OM_{—}10 mRNA or protein.

The methods of the invention can also be used to detect naturally occurring genetic mutations in an UP_{—}11 or OM_{—}10 gene, thereby determining if a subject with the mutated gene is at risk for a disorder characterized by aberrant or abnormal UP_{—}11 or OM_{—}10 nucleic acid expression or UP_{—}11 or OM_{—}10 polypeptide activity as described herein. In preferred embodiments, the methods include detecting, in a sample of cells from the subject, the presence or absence of a genetic mutation characterized by at least one of an alteration affecting the integrity of a gene encoding an UP_{—}11 or OM_{—}10 polypeptide, or the misexpression of the UP_{—}11 or OM_{—}10 gene. For example, such genetic mutations can be detected by ascertaining the existence of at least one of 1) a deletion of one or more nucleotides from an UP_{—}11 or OM_{—}10 gene; 2) an addition of one or more nucleotides to an UP_{—}11 or OM_{—}10 gene; 3) a substitution of one or more nucleotides of an UP_{—}11 or OM_{—}10 gene, 4) a chromosomal rearrangement of an UP_{—}11 or OM_{—}10 gene; 5) an alteration in the level of a messenger RNA transcript of an UP_{—}11 or OM_{—}10 gene, 6) aberrant modification of an UP_{—}11 or OM_{—}10 gene, such as of the methylation pattern of the genomic DNA, 7) the presence of a non-wild type splicing pattern of a messenger RNA transcript of an UP_{—}11 or OM_{—}10 gene, 8) a non-wild type level of an UP_{—}11 or OM_{—}10-protein, 9) allelic loss of an UP_{—}11 or OM_{—}10 gene, and 10) inappropriate post-translational modification of an UP_{—}11 or OM_{—}10-protein. As described herein, there are a large number of assay techniques known in the art that can be used for detecting mutations in an UP_{—}11 or OM_{—}10 gene.

In certain embodiments, detection of the mutation involves the use of a probe/primer in a polymerase chain reaction (PCR) (see, e.g. U.S. Pat. No. 4,683,195 and U.S. Pat. No. 4,683,202), such as anchor PCR or RACE PCR, or, alternatively, in a ligation chain reaction (LCR), the latter of which can be particularly useful for detecting point mutations in the UP_{—}11 or OM_{—}10-gene. This method can include the steps of collecting a sample of cells from a patient, isolating nucleic acid (e.g., genomic, mRNA or both) from the cells of the sample, contacting the nucleic acid sample with one or more primers which specifically hybridize to an UP_{—}11 or OM_{—}10 gene under conditions such that hybridization and amplification of the UP_{—}11 or OM_{—}10-gene (if present) occurs, and detecting the presence or absence of an amplification product, or detecting the size of the amplification product and comparing the length to a control sample.

In an alternative embodiment, mutations in an UP_{—}11 or OM 10 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see U.S. Pat. No. 5,498,531 hereby incorporated by reference in its entirety) can be used to score for the presence of specific mutations by development or loss of a ribozyme cleavage site.

In yet another embodiment, any of a variety of sequencing reactions known in the art can be used to directly sequence the UP_{—}11 or OM_{—}10 gene and detect mutations by comparing the sequence of the sample UP_{—}11 or OM_{—}10 gene with the corresponding wild-type (control) sequence. Examples of sequencing reactions include those based on techniques developed by Maxim and Gilbert (1977) or Sanger (1977). A variety of automated sequencing procedures can be utilized when performing the diagnostic assays, including sequencing by mass spectrometry (see, e.g., International Application No. WO 94/16101; Cohen et al., 1996; and Griffin et al. 1993).

Other methods for detecting mutations in the UP_{—}11 or OM_{—}10 gene include methods in which protection from cleavage agents is used to detect mismatched bases in RNA/RNA or RNA/DNA duplexes (Myers et al., 1985(a); Cotton et al., 1988; Saleeba et al., 1992), electrophoretic mobility of mutant and wild type nucleic acid is compared (Orita et al., 1989; Cotton, 1993; and Hayashi, 1992), and movement of mutant or wild-type fragments in polyacrylamide gels containing a gradient of denaturant is assayed using denaturing gradient gel electrophoresis (Myers et al., 1985). Examples of other techniques for detecting point mutations include, selective oligonucleotide hybridization, selective amplification, and selective primer extension.

Methods of Treatment

Another aspect of the invention pertains to methods for treating a subject, e.g., a human, having a disease or disorder characterized by (or associated with) aberrant or abnormal UP_{—}11 or OM_{—}10 nucleic acid expression and/or UP_{—}11 or OM_{—}10 polypeptide activity. These methods include the step of administering an UP_{—}11 or OM_{—}10 polypeptide/gene modulator (agonist or antagonist) to the subject such that treatment occurs. The language “aberrant or abnormal UP_{—}11 or OM_{—}10 polypeptide expression” refers to expression of a non-wild-type UP_{—}11 or OM_{—}10 polypeptide or a non-wild-type level of expression of an UP_{—}11 or OM_{—}10 polypeptide. Aberrant or abnormal UP_{—}11 or OM_{—}10 polypeptide activity refers to a non-wild-type UP_{—}11 or OM_{—}10 polypeptide activity. As the UP_{—}11 or OM_{—}10 polypeptide is involved in a pathway involving signaling within cells, aberrant or abnormal UP_{—}11 or OM_{—}10 polypeptide activity or expression interferes with the normal regulation of functions mediated by UP_{—}11 or OM_{—}10 polypeptide signaling. The terms “treating” or “treatment,” as used herein, refer to reduction or alleviation of at least one adverse effect or symptom of a disorder or disease, e.g., a disorder or disease characterized by or associated with abnormal or aberrant UP_{—}11 or OM_{—}10 polypeptide activity or UP_{—}11 or OM_{—}10 nucleic acid expression.

As used herein, an UP_{—}11 or OM_{—}10 polypeptide/gene modulator is a molecule which can modulate UP_{—}11 or OM_{—}10 nucleic acid expression and/or UP_{—}11 or OM_{—}10 polypeptide activity. For example, an UP_{—}11 or OM_{—}10 gene or protein modulator can modulate, e.g., upregulate (activate/agonize) or downregulate (suppress/antagonize), UP_{—}11 or OM_{—}10 nucleic acid expression. In another example, an UP_{—}11 or OM_{—}10 polypeptide/gene modulator can modulate (e.g., stimulate/agonize or inhibit/antagonize) GPCR polypeptide activity. If it is desirable to treat a disorder or disease characterized by (or associated with) aberrant or abnormal (non-wild-type) UP_{—}11 or OM_{—}10 nucleic acid expression and/or UP_{—}11 or OM_{—}10 polypeptide activity by inhibiting UP_{—}11 or OM_{—}10 nucleic acid expression, an UP_{—}11 or OM_{—}10 modulator can be an antisense molecule, e.g., a ribozyme, as described herein. Examples of antisense molecules which can be used to inhibit UP_{—}11 or OM_{—}10 nucleic acid expression include antisense molecules which are complementary to a fragment of the 5′ untranslated region (which also includes the start codon) of SEQ ID NO:1, SEQ ID NO:2, SEQ ID NO:3, SEQ ID NO:5, SEQ ID NO:6, SEQ ID NO:8 and SEQ ID NO:10 and antisense molecules which are complementary to a fragment of a 3′ untranslated region of SEQ ID NO:1, SEQ ID NO:2, SEQ ID NO:3, SEQ ID NO:5, SEQ ID NO:6, SEQ ID NO:8 or SEQ ID NO:10.

An UP_{—}11 or OM_{—}10 modulator that inhibits UP_{—}11 or OM_{—}10 nucleic acid expression can also be a small molecule or other drug, e.g., a small molecule or drug identified using the screening assays described herein, which inhibits UP_{—}11 or OM_{—}10 nucleic acid expression. If it is desirable to treat a disease or disorder characterized by (or associated with) aberrant or abnormal (non-wild-type) UP_{—}11 or OM_{—}10 nucleic acid expression and/or UP_{—}11 or OM_{—}10 polypeptide activity by stimulating UP_{—}11 or OM_{—}10 nucleic acid expression, an UP_{—}11 or OM_{—}10 modulator can be, for example, a nucleic acid molecule encoding an UP_{—}11 or OM_{—}10 polypeptide (e.g., a nucleic acid molecule comprising a nucleotide sequence homologous to the nucleotide sequence of SEQ ID NO:1, SEQ ID NO:2, SEQ ID NO:3, SEQ ID NO:5, SEQ ID NO:6, SEQ ID NO:8 or SEQ ID NO:10 or a small molecule or other drug, e.g., a small molecule (peptide) or drug identified using the screening assays described herein, which stimulates UP_{—}11 or OM_{—}10 nucleic acid expression.

Alternatively, if it is desirable to treat a disease or disorder characterized by (or associated with) aberrant or abnormal (non-wild-type) UP_{—}11 or OM_{—}10 nucleic acid expression and/or UP_{—}11 or OM_{—}10 polypeptide activity by inhibiting UP_{—}11 or OM_{—}10 polypeptide activity, an UP_{—}11 or OM_{—}10 modulator can be an anti-UP_{—}11 or anti-OM_{—}10 antibody or a small molecule or other drug, e.g., a small molecule or drug identified using the screening assays described herein, which inhibits UP_{—}11 or OM_{—}10 polypeptide activity. If it is desirable to treat a disease or disorder characterized by (or associated with) aberrant or abnormal (non-wild-type) UP_{—}11 or OM_{—}10 nucleic acid expression and/or UP_{—}11 or OM_{—}10 polypeptide activity by stimulating UP_{—}11 or OM_{—}10 polypeptide activity, an UP_{—}11 or OM_{—}10 modulator can be an active UP_{—}11 or OM_{—}10 polypeptide or fragment thereof (e.g., an UP_{—}11 or OM_{—}10 polypeptide or fragment thereof having an amino acid sequence which is homologous to the amino acid sequence of SEQ ID NO:4, SEQ ID NO:7, SEQ ID NO:9 or SEQ ID NO:11 or a fragment thereof) or a small molecule or other drug, e.g., a small molecule or drug identified using the screening assays described herein, which stimulates UP_{—}11 or OM_{—}10 polypeptide activity.

Other aspects of the invention pertain to methods for modulating an UP_{—}11 or OM_{—}10 polypeptide mediated cell activity. These methods include contacting the cell with an agent (or a composition which includes an effective amount of an agent) which modulates UP_{—}11 or OM_{—}10 polypeptide activity or UP_{—}11 or OM_{—}10 nucleic acid expression such that an UP_{—}11 or OM_{—}10 polypeptide mediated cell activity is altered relative to normal levels (for example, cAMP or phosphatidylinositol metabolism). As used herein, “a GPCR polypeptide mediated cell activity” or “an UP_{—}11 or OM_{—}10 polypeptide mediated cell activity” refers to a normal or abnormal activity or function of a cell. Examples of UP_{—}11 or OM_{—}10 polypeptide mediated cell activities include phosphatidylinositol turnover, production or secretion of molecules, such as proteins, contraction, proliferation, migration, differentiation, and cell survival. The term “altered” as used herein refers to a change, e.g., an increase or decrease, of a cell associated activity particularly cAMP or phosphatidylinositol turnover, and adenylate cyclase or phospholipase C activation.

In one embodiment, the agent stimulates UP_{—}11 or OM_{—}10 polypeptide activity or UP_{—}11 or OM_{—}10 nucleic acid expression. In another embodiment, the agent inhibits UP_{—}11 or OM_{—}10 polypeptide activity or UP_{—}11 or OM_{—}10 nucleic acid expression. These modulatory methods can be performed in vitro (e.g., by culturing the cell with the agent) or, alternatively, in vivo (e.g., by administering the agent to a subject). In a preferred embodiment, the modulatory methods are performed in vivo, i.e., the cell is present within a subject, e.g., a mammal, e.g., a human, and the subject has a disorder or disease characterized by or associated with abnormal or aberrant UP_{—}11 or OM_{—}10 polypeptide activity or UP_{—}11 or OM_{—}10 nucleic acid expression.

A nucleic acid molecule, a protein, an UP_{—}11 or OM_{—}10 modulator, a compound etc. used in the methods of treatment can be incorporated into an appropriate pharmaceutical composition described below and administered to the subject through a route which allows the molecule, protein, modulator, or compound etc. to perform its intended function.

A modulator of UP_{—}11 or OM_{—}10 polynucleotide expression and/or UP_{—}11 or OM_{—}10 polypeptide activity may be used in the treatment of various diseases or disorders including, but not limited to, the cardiopulmonary system such as acute heart failure, hypotension, hypertension, angina pectoris, myocardial infarction and the like; the gastrointestinal system; the central nervous system; kidney diseases; liver diseases; hyperproliferative diseases, such as cancers and psoriasis; apoptotic diseases; pain; endometriosis; anorexia; bulimia; asthma; osteoporosis; neuropsychiatric disorders such as schizophrenia, delirium, bipolar, depression, anxiety, panic disorders; urinary retention; ulcers; allergies; benign prostatic hypertrophy; and dyskinesias, such as Huntington's disease or Gilles dela Tourett's syndrome Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including FHV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degeneration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia; degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Elizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B1) deficiency and vitamin B12 deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastorna multiforme, pilocytic astrocytoma, pleomorphic xanthoastrocytorna, and brain stem glioma, oligodendrogliorna, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), neurocutaneous syndromes (phakomatoses), including neurofibromotosis, including Type I neurofibromatosis (NFI) and TYPE 2 neurofibromatosis (NF2), tuberous sclerosis, and Von Hippel-Lindau disease, and neuropsychiatric disorders, such as schizophrenia, bipolar, depression, anxiety and panic disorders.

Pharmacogenomics

Test/candidate compounds, or modulators which have a stimulatory or inhibitory effect on UP_{—}11 or OM_{—}10 polypeptide activity (e.g., UP_{—}11 or OM_{—}10 gene expression) as identified by a screening assay described herein can be administered to individuals to treat (prophylactically or therapeutically) disorders (e.g., neurological disorders) associated with aberrant UP_{—}11 or OM_{—}10 polypeptide activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permit the selection of effective compounds (e.g., drugs) for prophylactic or therapeutic treatments based on a consideration of the individual's genotype. Such pharmacogenomics can further be used to determine appropriate dosages and therapeutic regimens. Accordingly, the activity of UP_{—}11 or OM_{—}10 polypeptide, expression of UP_{—}11 or OM_{—}10 nucleic acid, or mutation content of UP_{—}11 or OM_{—}10 genes in an individual can be determined to thereby select appropriate compound(s) for therapeutic or prophylactic treatment of the individual.

Pharmacogenomics deal with clinically significant hereditary variations in the response to drugs due to altered drug disposition and abnormal action in affected persons. See, e.g., Eichelbaum, 1996 and Linder, 1997. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (GOD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2136 and CYP2C19) has provided an explanation as to why some patients do not obtain the expected drug effects or show exaggerated drug response and serious toxicity after taking the standard and safe dose of a drug.

These polymorphisms are expressed in two phenotypes in the population, the extensive metabolizer (EM) and poor metabolizer (PM). The prevalence of PM is different among different populations. For example, the gene coding for CYP2136 is highly polymorphic and several mutations have been identified in PM, which all lead to the absence of functional CYP2D6. Poor metabolizers of CYP2136 and CYP2C19 quite frequently experience exaggerated drug response and side effects when they receive standard doses.

If a metabolite is the active therapeutic moiety, PMs show no therapeutic response, as demonstrated for the analgesic effect of codeine mediated by its CYP2136-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of UP_{—}11 or OM_{—}10 polypeptide, expression of UP_{—}11 or OM_{—}10 nucleic acid, or mutation content of UP_{—}11 or OM_{—}10 genes in an individual can be determined to thereby select appropriate agent(s) for therapeutic or prophylactic treatment of a subject. In addition, pharmacogenetic studies can be used to apply genotyping of polymorphic alleles encoding drug-metabolizing enzymes to the identification of a subject's drug responsiveness phenotype. This knowledge, when applied to dosing or drug selection, can avoid adverse reactions or therapeutic failure and thus enhance therapeutic or prophylactic efficiency when treating a subject with an UP_{—}11 or OM_{—}10 modulator, such as a modulator identified by one of the exemplary screening assays described herein.

Monitoring of Effects During Clinical Trials

Monitoring the influence of compounds (e.g., drugs) on the expression or activity of UP_{—}11 or OM_{—}10 polypeptide/gene can be applied not only in basic drug screening, but also in clinical trials. For example, the effectiveness of an agent determined by a screening assay, as described herein, to increase UP_{—}11 or OM_{—}10 gene expression, protein levels, or up-regulate UP_{—}11 or OM_{—}10 activity, can be monitored in clinical trials of subjects exhibiting decreased UP_{—}11 or OM_{—}10 gene expression, protein levels, or down-regulated UP_{—}11 or OM_{—}10 polypeptide activity. Alternatively, the effectiveness of an agent, determined by a screening assay, to decrease UP_{—}11 or OM_{—}10 gene expression, protein levels, or down-regulate UP_{—}11 or OM_{—}10 polypeptide activity, can be monitored in clinical trials of subjects exhibiting increased UP_{—}11 or OM_{—}10 gene expression, protein levels, or up-regulated UP_{—}11 or OM_{—}10 polypeptide activity. In such clinical trials, the expression or activity of an UP_{—}11 or OM_{—}10 polypeptide and, preferably, other genes which have been implicated in, for example, a nervous system related disorder can be used as a “read out” or markers of the ligand responsiveness of a particular cell.

For example, and not by way of limitation, genes, including an UP_{—}11 or OM_{—}10 gene, which are modulated in cells by treatment with a compound (e.g., drug or small molecule) which modulates UP_{—}11 or OM_{—}10 polypeptide/gene activity (e.g., identified in a screening assay as described herein) can be identified. Thus, to study the effect of compounds on CNS disorders, for example, in a clinical trial, cells can be isolated and RNA prepared and analyzed for the levels of expression of an UP_{—}11 or OM_{—}10 gene and other genes implicated in the disorder. The levels of gene expression (i.e., a gene expression pattern) can be quantified by Northern blot analysis or RT-PCR, as described herein, or alternatively by measuring the amount of protein produced, by one of the methods described herein, or by measuring the levels of activity of an UP_{—}11 or OM_{—}10 polypeptide or other genes. In this way, the gene expression pattern can serve as an marker, indicative of the physiological response of the cells to the compound. Accordingly, this response state may be determined before, and at various points during, treatment of the individual with the compound.

In a preferred embodiment, the present invention provides a method for monitoring the effectiveness of treatment of a subject with a compound (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the compound; (ii) detecting the level of expression of an UP_{—}11 or OM_{—}10 polypeptide, mRNA, or genomic DNA in the preadministration sample; (iii) obtaining one or more post-administration samples from the subject; (iv) detecting the level of expression or activity of the UP_{—}11 or OM_{—}10 polypeptide, mRNA, or genomic DNA in the post-administration samples; (v) comparing the level of expression or activity of the UP_{—}11 or OM_{—}10 polypeptide, mRNA, or genomic DNA in the pre-administration sample with the UP_{—}11 or OM 10 polypeptide, mRNA, or genomic DNA in the post administration sample or samples; and (vi) altering the administration of the compound to the subject accordingly. For example, increased administration of the compound may be desirable to increase the expression or activity of an UP_{—}11 or OM_{—}10 polypeptide/gene to higher levels than detected, i.e., to increase the effectiveness of the agent.

Alternatively, decreased administration of the agent may be desirable to decrease expression or activity of UP_{—}11 or OM_{—}10 to lower levels than detected, i.e. to decrease the effectiveness of the compound.

Pharmaceutical Compositions

The UP_{—}11 or OM_{—}10 nucleic acid molecules, UP_{—}11 or OM_{—}10 polypeptides (particularly fragments of UP_{—}11 or OM_{—}10), modulators of an UP_{—}11 or OM_{—}10 polypeptide, and anti-UP_{—}11 or OM_{—}10 antibodies (also referred to herein as “active compounds”) of the invention can be incorporated into pharmaceutical compositions suitable for administration to a subject, e.g., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral (e.g., intravenous, intradermal, subcutaneous), oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an UP_{—}11 or OM_{—}10 polypeptide or anti-UP_{—}11 or OM_{—}10 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated by reference herein in its entirety.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. The pharmaceutical compositions can be included in a container, pack, or dispenser together with instructions for administration.

G. Uses of Partial UP_{—}11 or OM_{—}10 Sequences

Fragments or fragments of the cDNA sequences identified herein (and the corresponding complete gene sequences) can be used in numerous ways as polynucleotide reagents. For example, these sequences can be used to: (a) map their respective genes on a chromosome; and, thus, locate gene regions associated with genetic disease; (b) identify an individual from a minute biological sample (tissue typing); and (c) aid in forensic identification of a biological sample. These applications are described in the subsections below.

Chromosome Mapping

The mapping of the UP_{—}11 or OM_{—}10 sequence to chromosomes is an important first step in correlating these sequence with genes associated with disease. The UP_{—}11 sequence maps to chromosome 1 μl and OM_{—}10 maps to Xq27. The relationship between genes and disease, mapped to the same chromosomal region, can then be identified through linkage analysis (co-inheritance of physically adjacent genes).

Moreover, differences in the DNA sequences between individuals affected and unaffected with a disease associated with the UP_{—}11 or OM_{—}10 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### EXAMPLES

The following examples are carried out using standard techniques, which are well known and routine to those of skill in the art, except where otherwise described in detail. The following examples are presented for illustrative purpose, and should not be construed in any way as limiting the scope of this invention.

### Example 1

### Identification of Human UP_{—}11 and OM_{—}10 Polynucleotide Sequences

A TBLASTN (Altschul et al., 1997) search against the High Throughput Genomic Sequences (HTGS) section of Genbank, and against the Celera Human Genome Database was performed using the human 5-HT₆ receptor sequence (Accession Number L41147), in order to identify novel GPCR-like genes. The resulting HSPs were parsed, assembled and re-blasted using BLASTP versus a comprehensive protein database. This secondary BLAST search was used to identify novel GPCR-encoding genomic sequences. Novel sequences were further analyzed using Genscan (Burge and Karlin, 1997), and BLAST homologies to predict putative novel GPCRs. The predicted human sequences were used to design oligonucleotide primers used in obtaining human UP_{—}11 and OM_{—}10 physical clones.

BLAST queries were also performed on the Celera Mouse Genome database using the predicted sequences of human UP_{—}11 and OM_{—}10. Celera Mouse fragments with high similarity to UP_{—}11 and OM_{—}10 were assembled using Sequencher (GeneCodes) and Genscan was used to predict the mouse open reading frame.

Physical cDNA clones (human UP_{—}11 isolate 179 (SEQ ID NO:1), human UP_{—}11 isolate 200 (SEQ ID NO:2) and human UP_{—}11 isolate 30 (SEQ ID NO:3); mouse mUP_{—}11 isolate 67.1 (SEQ ID NO:5) and mouse mUP_{—}11 isolate 52.1 (SEQ ID NO:6); human OM_{—}10 (SEQ ID NO:8) and mouse mOM_{—}10 (SEQ ID NO:10)) were isolated from cDNA libraries as described below.

### Example 2

### Methods Used in Cloning UP_{—}11 and OM_{—}10

Library Construction

Plasmid cDNA libraries L600C, L601C and L701C were constructed using Clontech PolyA RNA (Human Brain, Hippocampus (catalog # 6578-1), Human Brain, Amygdala (catalog # 6574-1), and Mouse Brain (catalog # 6616-1) respectively) and Life Technologies SuperScript Plasmid System for cDNA Synthesis and Plasmid Cloning kit (catalog no. 18248-013). The manufacturer's protocol was followed with three modifications: (1) In both first and second strand synthesis reactions, DEPC-treated water was substituted for (alpha P³²)dCTP. (2) The Sal I-adapted cDNA was size-fractionated by gel electrophoresis on 1% agarose, 0.1 ug/ml ethidium bromide, 1×TAE gels. The ethidium bromide-stained cDNA ≧3.0 kb was excised from the gel. The cDNA was purified from the agarose gel by electroelution (ISCO Little Blue Tank Electroelutor and protocol). (3) The gel-purified, size-fractionated Sal I-adapted cDNA was ligated to NotI-SalI digested pCMV-SPORT6 (Life Technologies, Inc., L600, L601) or pBluescript SK (Stratagene, L701).

Plasmid Construction: Human UP 11

Plasmid pCR112HUP11_{—}12B, which contains the sequence of the predicted human UP 11 gene, was constructed as described below.

Polymerase chain reaction (PCR) amplification was performed using standard techniques. A reaction mixture was compiled with components at the following final concentrations: 0.1 ug of human genomic DNA (Clonetech, catalog no. 6550-1);

10 pmol of forward primer

5′ATGCATGCAAGCTTGCACCATGCTCCTGCTGGACTTGACTGC (SEQ ID NO:22);

10 pmol of reverse primer

5′ATGCATGCCTCGAGTGACTCCAGCCGGGGTGA (SEQ ID NO:23);

0.2 mM each dATP, dTTP, dCTP, and dGTP (Amersham Pharmacia Biotech catalog no. 27-2094-01); 1.5 units Taq DNA polymerase; 1×PCR reaction buffer (20 mM Tris-HCl (pH 8.4), 50 mM KCl, Life Technologies, catalog no.10342); 0.15 mM MgCl₂. The mixture was incubated at 94° C. for one minute, followed by 35 cycles of 94° C. for 30 seconds, 65 C for 25 seconds, 72° C. for 70 seconds, followed by a final incubation at 72° C. for five minutes (MJResearch DNA Engine Tetrad PTC-225).

The PCR reaction products (“DNA”) were size-fractionated by gel electrophoresis on 1% agarose, 0.1 ug/ml ethidium bromide, 0.5×TBE gels (Maniatis et al., 1982). The ethidium bromide-stained DNA band of the appropriate size was excised from the agarose gel. The DNA was extracted from the agarose using the Clonetech NucleoSpin Nucleic Acid Purification Kit (catalog no. K3051-2) and manufacturer's protocol. Subsequently, the DNA was sub-cloned into the vector pCRII-TOPO using the Invitrogen TOPO TA Cloning kit (Invitrogen catalog no. K4600) and manufacturer's protocol with modifications. Briefly, approximately 40 ng of the gel purified PCR product was incubated with one ul of the manufacturer supplied pCR11-TOPO DNA (10 ng/ul), and one ul of diluted Salt Solution 0.3M NaCl, 0.15M MgCl₂) in a final volume of six ul. The mixture was incubated for five minutes at room temperature (approximately 25° C.). One ul of this reaction was added to electocompetent cells (ElectroMAX DH10B cells, Life Technologies catalog no. 18290-015) and electoporated using the Biorad E. coli pulser (voltage 1.8 KV, 3-5 msec pulse). One ml of LB (Sambrook et al, 1989) was added to the cells and the mixture incubated at 37° C. for 1.5 hours. The mixture was plated on LB-ampicillin agar plates and incubated overnight at 37° C. Bacterial clones containing the predicted human UP_{—}11 sequence were identified by restriction digestion analysis (standard molecular techniques) and sequence analysis (ABI Prism BigDye Terminator Cycle Sequencing, catalog no. 4303154, ABI 377 instruments) of plasmid DNA prepared from isolated colonies. Plasmid DNA was prepared using the QIAprep Spin Miniprep Kit and protocol (Qiagen Inc, catalog no. 27106).

Plasmid Construction: Human OM 10

Plasmid pCRII2KOM10_{—}6B, which contains the sequence of the predicted human OM 10 gene, was constructed as described above for Human UP_{—}11 with the following modifications.

The OM_{—}10 forward primer was 5′ATGCATGCAAGCTTGCACCATGACGTCCACCTGCACCAACAG (SEQ ID NO:24) and the reverse primer was 5′ ATGCATGCCTCGAGAGGAAAAGTAGCAGAATCG (SEQ ID NO:25).

Isolation of Human UP 11 cDNA Clones 179, 200, 30

UP_{—}11 cDNA clones 179 (SEQ ID NO:1), 200 (SEQ ID NO:2), and 30 (SEQ ID NO:3 were isolated by screening approximately 2,000,000 primary transformants from plasmid cDNA library L601C with a P³² labeled DNA probe using standard molecular biology techniques. Colony lift hybridizations were performed at 68° C. in 5× Denhardt's, 5×SSC, 1% SDS, 100 ug/ml denatured salmon sperm. The colony lifts were washed at 60° C. in 0.1×SSC, 1% SDS. Probe generation is described below. Plasmid DNA, prepared as described above, from isolated positively hybridizing colonies from L601C was analyzed by restriction digestion analysis and sequence analysis (ABI Prism BigDye Terminator Cycle Sequencing, catalog no. 4303154, ABI 377 instruments). cDNA clones 179, 30 and 200 contained the predicted UP_{—}11 open reading frame.

Probe Generation

The human UP_{—}11 specific probe used in the library screen was generated as follows. Plasmid DNA from pCRII2HUP11_{—}12B was restriction digested with EcoRI (New England Biolabs, catalog no. 101) according to the manufacturer's protocol. Restriction fragments were size-fractionated by gel electrophoresis on 1.5% agarose, 0.1 ug/ml ethidium bromide, 1×TAE gels. The ethidium bromide-stained DNA band of the appropriate size (approximately 1200 bp) was excised from the agarose gel. Next, the DNA was extracted from the agarose using the Clonetech NucleoSpin Nucleic Acid Purification Kit (catalog no. K3051-2) and manufacturer's protocol. The extracted DNA was labeled with Redivue (alpha P³²)dCTP (Amersham Pharmacia, catalog no. AA0005) using the Prime-It II Random Primer Labeling Kit and protocol (Stratagene, catalog no. 300385). Unincorporated (alpha P³²)dCTP was removed with Amersham's NICK column and protocol (catalog no. 17-0855-O₂)

Isolation of Human OM 10 Clone

A human OM_{—}10 cDNA clone (SEQ ID NO:8) was isolated as described above in human UP_{—}11 cDNA clone isolation with the following modifications. (1) 2,000,000 primary transformants of library L600C were screened and a single clone containing the predicted human OM_{—}10 open reading frame was isolated. (2) The library was screened with the approximately 1500 bp EcoRI restriction fragment from plasmid pCRII2KOM10_{—}6B.

Isolation of Mouse OM 10 and UP 11 Clones

Mouse UP_{—}11 and OM_{—}10 cDNA clones were isolated as described for human UP_{—}11 cDNA clone isolation with the following modifications. (1) For each gene, 2,000,000 primary transformants of library L701C were screened. (2) Colony lift hybridizations were performed at 60° C. in 5× Denhardt's, 4×SSC, 1% SDS, 100 ug/ml denatured salmon sperm. The colony lifts were washed at 60 C in 0.25×SSC, 1% SDS. (3) The lifts were probed with the approximately 1200 bp EcoRI restriction fragment of plasmid pCRII2HUP11_{—}12B or the approximately 1500 bp EcoRI restriction fragment from plasmid pCRII2KOM10_{—}6B.

In the UP_{—}11 screen, two positively hybridizing colonies were identified, isolates 67.1 (SEQ ID NO:5) and 52.1 (SEQ ID NO:6); and both contained the mouse UP_{—}11 open reading frame as was predicted from the TblastN query of the Celera mouse genome. In the OM_{—}10 screen, a single positively hybridizing colony was identified (SEQ ID NO:10), and it contained the mouse OM_{—}10 open reading frame as was predicted from the TblastN query of the Celera mouse genome.

### Example 3

### Tissue Expression of the Human and Mouse UP_{—}11 and OM_{—}10 Genes

Human UP_{—}11 and OM_{—}10

To assess the tissue distribution of the human UP_{—}11 and OM_{—}10 genes, Northern analysis was performed using blots containing 1 ug of poly A+ RNA per lane isolated from various human tissues (catalog no. 7780-1 and 7755-1, Clontech, Palo Alto, Calif.) and probed with a human UP_{—}11 or OM_{—}10-specific probe. The filters were prehybridized in 10 ml of Express Hyb hybridization solution (Clontech, Palo Alto, Calif.) at 68° C. for 1 hour, after which approximately 100 ng of ³²P labeled probe was added. The probe was generated using the Stratagene Prime-It kit, Catalog Number 300392 (Clontech, Palo Alto, Calif.).

The human UP_{—}11 specific P³² labeled DNA probe contained nucleotides 442-1653 of the human UP_{—}11 sequence in SEQ ID NO:1. The human OM_{—}10 specific P³² labeled DNA probe contained nucleotides 332-1,858 of the human OM_{—}10 sequence in SEQ ID NO:8. Using the human UP_{—}11 specific probe, transcripts of approximately 3 kb, 4.4 kb and 8 kb were strongly detected in whole brain tissue and weakly detected in skeletal muscle on the Human 12-Lane Multiple Tissue Northern (7780). A transcript was not detected in other tissues on this Northern. Transcripts of the same size were detected on the Brain II MTN (7755) in cerebellum, cerebral cortex, medulla, occipital pole, frontal lobe, temporal lobe and putamen. The expression of human UP_{—}11 was further analyzed with Human Multiple Tissue Expression Array (catalog no. 7775-1, user manual PT3307-1) membranes. Hybridization to poly(A)+ RNA from multiple tissues was detectable on the Human Multiple Tissue Expression Array: strong hybridization to fetal brain, whole brain, cerebral cortex, frontal lobe, parietal lobe, occipital lobe, temporal lobe, paracentral gyrus of cerebral cortex, pons, left and right cerebellum, hippocampus, medulla oblongata, putamen, accumbens and pituitary gland; moderate hybridization to corpus callosum, amygdala, caudate nucleus, substantia nigra, and thalamus, and weak hybridization to spinal cord. There was no hybridization detected in other tissues on this array.

Using the human OM_{—}10-specific probe, no transcript was detected in any of the tissues on the Human 12-Lane Multiple Tissue Northern (7780). On the Brain II MTN (7755), there was strong hybridization to two transcripts, approximately 8 kb and 4 kb, in putamen. Weaker hybridization was seen to the approximately 8 kb transcript in cerebellum, cerebral cortex, and medulla. A transcript was not detected in other tissues on this Northern. The expression of human OM_{—}10 was further analyzed with Human Multiple Tissue Expression Array (catalog no. 7775-1, user manual PT3307-1) membranes. Hybridization to poly(A)+ RNA from multiple tissues was detectable on the Human Multiple Tissue Expression Array: strong hybridization to putamen and caudate nucleus and weak hybridization to medulla oblongata, hippocampus and amygdala. There was no hybridization detected in other tissues on this array.

Mouse UP 11 and OM 10

To assess the tissue distribution of the mouse UP_{—}11 and OM_{—}10 transcripts, Northern analysis was performed using blots containing 1 ug of poly A+ RNA per lane isolated from various mouse tissues (catalog no. 7762-1), Clontech, Palo Alto, Calif.) probed with a mouse UP_{—}11- or OM_{—}10-specific probe. The Clontech filters were prehybridized in 10 ml of Express Hyb hybridization solution (Clontech, Palo Alto, Calif.) at 68° C. for 1 hour, after which 100 ng of P³² labeled probe was added. The probe was generated using the Stratagene Prime-It kit, Catalog Number 300392 (Clontech, Palo Alto, Calif.). Mouse MTN Blot (catalog no. 7762-1)

Tissue distribution within the mouse brain was assessed by Northern analysis as follows. Defined regions of the mouse brain (strain 129Sv or Balb/c) were micro-dissected and immediately frozen on dry ice. Total RNA was isolated from the frozen tissue using Triazol (Gibco, 15596) and the manufacturer's protocol. Total RNA was size-fractionated by electrophoresis on denaturing gels (7.4% formaldehyde, 1.1% agarose, 1×MOPS buffer (0.1 M MOPS, 5 mM sodium acetate, 1 mM EDTA)). RNA in sample buffer (62.5% formamide, 1.25×MOPS buffer) was incubated for 5 minutes at 65° C., formaldehyde was added to achieve a final concentration of 7.4%, followed by an additional 5 minutes at 65° C., and cooled on ice prior to electrophoresis. Approximately 10-15 ug of total RNA was loaded into each sample lane of the gel. The size-fractionated RNA was capillary blotted to Hybond N+nylon membranes overnight with 20×SSC. Subsequently, blots were rinsed in water and UV cross-linked. Blots were incubated in Quickhyb buffer (Stratagene) with 20 ug/ml of denatured, sonicated salmon sperm DNA (dSS) for 15 minutes at 65° C. Next, blots were incubated in Quickhyb with 25 ug/ml dSS and 50 ng of p³² labeled probe, synthesized as described above, for 2 hours at 65° C. Blots were washed twice, 10 minutes each, in 2×SSC, 1%SDS, at 65° C. Next, blots were washed twice, 20 minutes each, in 0.1×SSC, 1% SDS. The final two washes were in 0.05×SSC, 1% SDS, 65° C., 45 minutes each. Blots were exposed to X-ray film

The mouse UP_{—}11 specific P³² labeled DNA probe contained nucleotides 684-2033 of the mouse UP_{—}11 sequence in SEQ ID NO:5. The mouse OM_{—}10 specific P³² labeled DNA probe contained nucleotides 1080-1780 of the mouse OM_{—}10 sequence in SEQ ID NO:10.

Using the mouse UP-11-specific probe, approximately 4 and 4.4 kb transcripts were detected in whole brain and multiple, weakly hybridizing transcripts (approximately 9.5 kb, approximately 4 kb, approximately 2 kb, approximately 1 kb) in testis on the Mouse MTN (7762). A transcript,was not detected in other tissues on this Northern. Two transcripts, approximately 4 kb and 4.4 kb, were detected in all mouse brain subregion tissues tested: olfactory bulb, striatum, cortex, hippocampus, colliculus, midbrain, and cerebellum.

Using the mouse OM_{—}10-specific probe, a single approximately 6 kb transcript was detected in whole brain on the Mouse MTN (7762). A transcript was not detected in other tissues on this Northern. A single approximately 6 kb transcript was detected in mouse brain subregions striatum, hypothalamus, colliculus, midbrain and the brain stem. No transcript was detected in the olfactory bulb, cortex, hippocampus, or cerebellum.

### Example 4

### Chromosomal Location of Human and Mouse OM_{—}10

Lymphocytes isolated from human blood were cultured in alpha-minimal essential medium (a-MEM) supplemented with 10% fetal calf serum and phytohemagglutinin at 37° C. for 68-72 hours. The lymphocyte cultures were treated with BrdU (0.18 mg/ml, Sigma) to synchronize the cell population. The synchronized cells were washed three times with serum free medium to release the block and recultured at 37° C. for 6 hours in MEM with thymidine (2.5 ug/ml; Sigma). Cells were harvested and slides were made by using standard procedures including hypotonic treatment, fixation and air-dried.

Mouse Chromosomal Slide Preparation

Monocytes were isolated from mouse spleen and cultured at 37° C. in RPMI 1640 medium supplemented with 15% fetal calf serum, 3 ug/ml concanavalin A, 10 ug/ml lipopolysaccharide and 5×10⁻⁵ M mercaptoethanol. After 44 hours, the cultured lymphocytes were treated with 0.18 mg/ml BrdU for an additional 14 hours. The synchronized cells were washed and recultured at 37° C. for 4 hours in a-MEM with thymidine (2.5 ug/ml). Chromosome slides were made by conventional method as used for human chromosome preparation (hypotonic treatment, fixation and air dry).

Probe Labelling, in situ Hybridization and Detection

DNA probes were biotinylated with dATP using the Gibco BRL BioNick labeling kit (15° C., 1 hour (Heng et al., 1992)). FISH detection was performed by SeeDNA Biotech (PO Box 21082, Windsor Ontario Canada) according to Heng et al., 1992; and Heng and Tsui, 1993. Briefly, slides were baked at 55° C. for 1 hour. After RNaseA treatment, the slides were denatured in 70% formamide in 2×SSC for 2 minutes at 70° C. followed by dehydration with ethanol. Probes were denatured at 75° C. for 5 minutes in a hybridization mix consisting of 50% formamide and 10% dextran sulphate. Probes were loaded on the denatured chromosomal slides. After overnight hybridization, slides were washed and detected as well as amplified using published method (Heng et al., 1992). FISH signals and the DAPI banding pattern were recorded separately. Images were captured and combined by CCD camera, and the assignment of the FISH mapping data with chromosomal bands was achieved by superimposing FISH signals with DAPI banded chromosomes (Heng and Tsui, 1993).

The approximately 4.7 kb NotI/SalI restriction fragment from the human OM_{—}10 cDNA clone was used as a probe on the human chromosomal slides. The approximately 5.3 kb NotI/SalI restriction fragment from the mouse OM_{—}10 cDNA clone was used as a probe on the mouse chromosomal slides

The mouse OM_{—}10 probe hybridized to mouse chromosome XA5. The human OM_{—}10 probe hybridized to human chromosome Xq26-q27. These chromosomal locations are likely syntenic regions, which supports our designation of these genes as orthologs (NCBI maps: Jackson Laboratory, Mouse Genome Informatics)

### Example 5

### Expression of Recombinant UP_{—}11 and OM_{—}10 Protein in Bacterial Cells

In this example, UP_{—}11 or OM_{—}10 is expressed as a recombinant glutathione-S-transferase (GST) fusion protein in E. coli and the fusion protein is isolated and characterized. Specifically, UP_{—}11 or OM_{—}10 is fused to GST and this fusion protein is expressed in E. coli, e.g., strain PEB 199. As the human UP_{—}11 and OM_{—}10 polypeptides are predicted to be approximately 49 kDa and 57 kDa, respectively, and GST is predicted to be 26 kDa, the fusion protein is predicted to be approximately 75 kDa and 83 kDa, in molecular weight. Expression of the GST-UP_{—}11 or OM_{—}10 fusion protein in PEB199 is induced with IPTG. The recombinant fusion protein is purified from crude bacterial lysates of the induced PEB 199 strain by affinity chromatography on glutathione beads.

Using polyacrylamide gel electrophoretic analysis of the protein purified from the bacterial lysates, the molecular weight of the resultant fusion protein may be determined.

### Example 6

### Expression of Recombinant UP_{—}11 and OM_{—}10 Protein in COS Cells

To express the UP_{—}11 or OM_{—}10 gene in COS cells, the pcDNA/Amp vector by Invitrogen Corporation (San Diego, Calif.) may be used. This vector contains an SV40 origin of replication, an ampicillin resistance gene, an E. coli replication origin, a CMV promoter followed by a polylinker region, and an SV40 intron and polyadenylation site. A DNA fragment encoding the entire UP_{—}11 or OM_{—}10 protein and a HA tag (Wilson et al., 1984) fused in-frame to the 3′ end of the fragment is cloned into the polylinker region of the vector, thereby placing the expression of the recombinant protein under the control of the CMV promoter.

To construct the plasmid, the UP_{—}11 or OM_{—}10 DNA sequence is amplified by PCR using two primers. The 5′ primer contains the restriction site of interest followed by approximately twenty nucleotides of the UP_{—}11 or OM_{—}10 coding sequence starting from the initiation codon; the 3′ end sequence contains complementary sequences to the other restriction site of interest, a translation stop codon, the HA tag and the last 20 nucleotides of the UP_{—}11 or OM_{—}10 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the UP_{—}11 or OM_{—}10 gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the UP_{—}11 or OM_{—}10-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook et al., 1989. The expression of the UP_{—}11 or OM_{—}10 protein is detected by radiolabelling (S³⁵-methionine or S³⁵-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with S³⁵-methionine (or S³⁵-cysteine). The culture media are then collected and the cells are lysed using detergents (RIPA buffer, 150 mM NaCl, 1%NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE. Alternatively, DNA containing the UP_{—}11 or OM_{—}10 coding sequence is cloned directly into the polylinker of the pcDNA/Amp vector using the appropriate restriction sites.

The resulting plasmid is transfected into COS cells in the manner described above, and the expression of the UP_{—}11 or OM_{—}10 protein is detected by radiolabelling and immunoprecipitation using an UP_{—}11 or OM_{—}10 specific monoclonal antibody.

### Example 7

### Expression of UP_{—}11 and OM_{—}10 in Mammalian Cells

Cell Line Generation

The open reading frame of human or mouse UP_{—}11 or OM_{—}10 is ligated into the mammalian expression vector pcDNA3.1+zeo (Invitrogen, 1600 Faraday Avenue, Carlsbad, Calif. 92008). HEK 293 cells are transfected with the plasmid and selected with 500 μg/ml zeocin. Zeocin resistant clones are tested for expression of UP_{—}11 or OM_{—}10 by RT-PCR and then tested for their ability to stimulate cAMP production.

Cyclase Assay

4×10⁵ cells are plated into 96 well Biocoat cell culture plates (Becton Dickinson, 1 Becton Drive, Franklin Lakes, N.J. 07417-1886) 24 hours prior to assay. The cells are then incubated in Krebs-bicarbonate buffer at 37° C. for 15 minutes. A 5 minute pretreatment with 500 μM isobutylmethyl xanthine (IBMX) precedes a 12 minute stimulation with 1 μM forskolin or buffer for determination,of basal cAMP levels. cAMP levels are determined using the SPA assay (Amersham Pharmacia Biotech, 800 Centennial Avenue, Pistcataway, NJ 08855).

### Example 8

### Characterization of the Human UP_{—}11 and OM_{—}10 Protein

In this example, the amino acid sequence of the human UP_{—}11 and OM_{—}10 protein was compared to amino acid sequences of known proteins and various motifs were identified. The human UP_{—}11 protein, the amino acid sequence of which is shown in SEQ ID NO:4, is a protein which includes 451 amino acid residues. The OM_{—}10 protein, the amino acid sequence of which is shown in SEQ ID NO:9, is a protein which includes 508 amino acid residues. Hydrophobicity analysis (FIG. 2) indicated that the human UP_{—}11 protein contains the expected 7 transmembrane domains and that they are located at amino acid residues: 11-16, 36-49, 69-83, 112-121, 160-182, 242-250 and 286-287 (Peak range, GvH scale, Toppred). Hydrophobicity analysis (FIG. 2) indicated that the human OM_{—}10 protein contains the expected 7 transmembrane domains and that they are located at amino acid residues: 34-49, 86-90, 109-118, 155-162, 188-214, 403-418 and 437-446 (Peak range, GvH scale, Toppred).

### Example 9

### Generation of ANTI-OM_{—}10 and UP_{—}11 Polyclonal Antibodies

Polyclonal antibodies directed against OM_{—}10 and UP_{—}11 peptide fragments were generated as follows: The OM_{—}10 peptides in Table 5 were synthesized, pooled and conjugated via the amino terminal cysteine to the carrier protein keyhole limpet hemocyanin (KLH). The UP_{—}11 peptides in Table 6 were synthesized, pooled and conjugated via the amino terminal cysteine to KLH. For each of the OM_{—}10 and UP_{—}11 projects, the conjugated, pooled immunogens were injected into New Zealand Rabbits with Complete Freund's Adjuvant. Subsequent boosts with peptides and Incomplete Freund's Adjuvant and bleeds were taken according to a standard ten-week protocol as is generally understood in the art. During the immunization schedule, ELISA titers of antisera were taken to determine adequacy of antibody formation. Western Blot Analysis demonstrated that the seras contained antibodies that could detect OM_{—}10 or UP_{—}11.

TABLE 5

Human OM_10 peptides used for generation of
polyclonal antisera

| | |
|---|---|
| CPLYGWGQAAFDERNA | (SEQ ID NO:12) |
| CVENEDEEGAEKKEE | (SEQ ID NO:13) |
| CQHEGEVKAKEGRMEA | (SEQ ID NO:14) |
| CSIDLGEDDMEFGED | (SEQ ID NO:15) |
| CMLKKFFCKEKPPKE | (SEQ ID NO:16) |

TABLE 6

Human UP_11 peptides used for generation of
polyclonal antisera

| | |
|---|---|
| CSSSALFDHALFGEVA | (SEQ ID NO:17) |
| CGAPQTTPHRTFGGG | (SEQ ID NO:18) |
| CFFKPAPEEELRLPS | (SEQ ID NO:19) |
| CKQEPPAVDFRIPGQIAE | (SEQ ID NO:20) |
| CLNRQIRGELSKQFV | (SEQ ID NO:21) |

### Example 10

### Construction of UP_{—}11 and OM_{—}10 Gene Targeting Vector

The murine UP_{—}11 or OM_{—}10 cDNA clone described in Example 2 is used as a probe to screen a genomic DNA library made from the 129 strain of mouse, again using standard techniques. The isolated murine UP_{—}11 or OM_{—}10 genomic clones are then subcloned into a plasmid vector, pBluescript (obtained commercially from Stratagene), for restriction mapping, partial DNA sequencing, and construction of the targeting vector. To functionally disrupt the UP_{—}11 or OM_{—}10 gene, a targeting vector may be prepared in which non-homologous DNA is inserted within the first coding exon, deleting the start codon and about 600 bp of UP_{—}11 or OM_{—}10 coding sequence (which would include the first 5 transmembrane domains) in the process and rendering the remaining downstream UP_{—}11 or OM_{—}10 coding sequences out of frame with respect to the start of translation. Therefore, if any translation products were to be formed from alternately spliced transcripts of the UP_{—}11 or OM_{—}10 gene, they would not contain all 7 transmembrane domains required for normal function of a GPCR. The UP_{—}11 or OM_{—}10 targeting vector is constructed using standard molecular cloning techniques. The targeting vector would contain 1-5 kb of murine UP_{—}11 or OM_{—}10 genomic sequence upstream of the initiating codon immediately followed by the neomycin phosphotransferase (neo) gene under the control of the phosphoglycerokinase promoter. Immediately downstream of the neomycin cassette is 1-5 kb of murine UP_{—}11 or OM_{—}10 genomic sequence corresponding to a region approximately 2 kb downstream of the murine UP_{—}11 or OM_{—}10 start codon. This is followed by the herpes simplex thymidine kinase (HSV tk) gene under the control of the phosphoglycerokinase promoter. The upstream and downstream genomic cassettes in this vector are in the same 5′ to 3′ orientation as the endogenous murine gene. The positive selection neo gene replaces the first coding exon of the UP_{—}11 or OM_{—}10 sequences and in the opposite orientation as the UP_{—}11 or OM_{—}10 gene, whereas the negative selection HSV tk gene is at the 3′ end of the construct. This configuration allowed for the use of the positive and negative selection approach for homologous recombination (Mansour et al., 1988). Prior to transfection into embryonal stem cells, the plasmid is linearized by restriction enzyme digestion.

### Example 11

### Transfection and Analysis of Embryonal Stem Cells

Embryonic stem cells (for example, strain D3, Doestschman et al., 1985) are cultured on a neomycin resistant embryonal fibroblast feeder layer grown in Dulbecco's Modified Eagles medium supplemented with 15% Fetal Calf Serum, 2 mM glutamine, penicillin (50 u/ml)/streptomycin (50 u/ml), non-essential amino acids, 100 uM 2-mercaptoethanol and 500 u/ml leukemia inhibitory factor. Medium is changed daily and cells are subcultured every two to three days and are then transfected with linearized plasmid, described in Example 10, by electroporation (25 uF capacitance and 400 Volts). The transfected cells are cultured in non-selective media for 1-2 days post transfection. Subsequently, they are cultured in media containing gancyclovir and neomycin for 5 days, of which the last 3 days are in neomycin alone. After expanding the clones, an aliquot of cells is frozen in liquid nitrogen. DNA is prepared from the remainder of cells for genomic DNA analysis to identify clones in which homologous recombination had occurred between the endogenous UP_{—}11 or OM_{—}10 gene and the targeting construct. To prepare genomic DNA, ES cell clones are lysed in 100 mM Tris HCl, pH 8.5, 5 mM EDTA, 0.2% SDS, 200 mM NaCl and 100 μg of proteinase K/ml. DNA is recovered by isopropanol precipitation, solubilized in 10 mM Tris HCl, pH 8.0, 0.1 mM EDTA. To identify homologous recombinant clones, genomic DNA isolated from the clones is digested with restriction enzymes. After restriction digestion, the DNA can be resolved on a 0.8% agarose gel, blotted onto a Hybond N membrane and hybridized at 65° C. with probes that bind a region of the UP_{—}11 or OM_{—}10 gene proximal to the 5′ end of the targeting vector and probes that bind a region of the UP_{—}11 or OM_{—}10 gene distal to the 3′ end of the targeting vector. After standard hybridization, the blots are washed with 40 mM NaPO4 (pH 7.2), 1 mM EDTA and 1% SDS at 65° C. and exposed to X-ray film. Hybridization of the 5′ probe to the wild type UP_{—}11 or OM_{—}10 allele results in a fragment readily discernible by autoradiography from the mutant UP_{—}11 or OM_{—}10 allele having the neo insertion.

### Example 12

### Generation of UP_{—}11 or OM_{—}10 Deficient Mice

Female and male mice are mated and blastocysts are isolated at 3.5 days of gestation. 10 to 12 cells from the clone described in Example 11 are injected per blastocyst and 7 or 8 blastocysts are transferred to the uterus of a pseudopregnant female. Pups are delivered by cesarean section on the 18th day of gestation and placed with a foster BALB/c mother. Resulting male and female chimeras are mated with female and male BALB/C mice (non-pigmented coat), respectively, and germline transmission is determined by the pigmented coat color derived from passage of 129 ES cell genome through the germline. The pigmented heterozygotes are likely to carry the disrupted UP_{—}11 or OM_{—}10 allele and therefore these animals are mated. Mendelian genetics predicts that approximately 25% of the offspring will be homozygous for the UP_{—}11 or OM_{—}10 null mutation. Genotyping of the animals is accomplished by obtaining tail genomic DNA.

To confirm that the UP_{—}11 or OM_{—}10 −/− mice do not express full-length UP_{—}11 or OM_{—}10 mRNA transcripts, RNA is isolated from various tissues and analyzed by standard Northern hybridizations with an UP_{—}11 or OM_{—}10 cDNA probe or by reverse transcriptase-polymerase chain reaction (RT-PCR). RNA is extracted from various organs of the mice using 4M Guanidinium thiocyanate followed by centrifugation through 5.7 M CsCl as described in Sambrook et al. (Molecular Cloning: A Laboratory Manual, 2nd Edition, Cold Spring Harbor Laboratory press (1989)). Northern analysis of UP_{—}11 or OM_{—}10 mRNA expression in brain or placenta will demonstrate that the full-length UP_{—}11 or OM_{—}10 mRNA is not detectable in brain or placenta from UP_{—}11 or OM_{—}10 −/− mice. Primers specific for the neomycin gene will detect a transcript in UP_{—}11 or OM_{—}10+/− and −/− but not +/+ animals. Northern and RT-PCT analyses are used to confirm that homozygous disruption of the UP_{—}11 or OM_{—}10 gene results in the absence of detectable full-length UP_{—}11 or OM_{—}10 mRNA transcripts in the UP_{—}11 or OM_{—}10 −/− mice. To examine UP_{—}11 or OM_{—}10 protein expression in the UP_{—}11 or OM_{—}10 deficient mice, Western blot analyses are performed on lysates from isolated tissue, including brain and placenta using standard techniques. These results will confirm that homozygous disruption of the UP_{—}11 or OM_{—}10 gene results in an absence of detectable UP_{—}11 or OM_{—}10 protein in the −/− mice.

### Example 13

### Inhibition of UP_{—}11 or OM_{—}10 Production

Design of RNA Molecules as Compositions of the Invention

All RNA molecules in this experiment are approximately 600 nts in length, and all RNA molecules are designed to be incapable of producing functional UP_{—}11 or OM_{—}10 protein. The molecules have no cap and no poly-A sequence; the native initiation codon is not present, and the RNA does not encode the full-length product. The following RNA molecules are designed:

(1) a single-stranded (ss) sense RNA polynucleotide sequence homologous to a portion of UP_{—}11 or OM_{—}10 murine messenger RNA (mRNA);

(2) a ss anti-sense RNA polynucleotide sequence complementary to a portion of UP_{—}11 or OM_{—}10 murine mRNA,

(3) a double-stranded (ds) RNA molecule comprised of both sense and anti-sense portion of UP_{—}11 or OM_{—}10 murine mRNA polynucleotide sequences,

(4) a ss sense RNA polynucleotide sequence homologous to a portion of UP_{—}11 or OM_{—}10 murine heterogeneous RNA (hnRNA),

(5) a ss anti-sense RNA polynucleotide sequence complementary to a portion of UP_{—}11 or OM_{—}10 murine hnRNA,

(6) a ds RNA molecule comprised of the sense and anti-sense UP_{—}11 or OM_{—}10 murine hnRNA polynucleotide sequences,

(7) a ss murine RNA polynucleotide sequence homologous to the top strand of the portion of UP_{—}11 or OM_{—}10 promoter,

(8) a ss murine RNA polynucleotide sequence homologous to the bottom strand of the portion of UP_{—}11 or OM_{—}10 promoter, and

(9) a ds RNA molecule comprised of murine RNA polynucleotide sequences homologous to the top and bottom strands of the UP_{—}11 or OM_{—}10 promoter.

The various RNA molecules of (1)-(9) above may be generated through T7 RNA polymerase transcription of PCR products bearing a T7 promoter at one end. In the instance where a sense RNA is desired, a T7 promoter is located at the 5′ end of the forward PCR primer. In the instance where an antisense RNA is desired, the T7 promoter is located at the 5′ end of the reverse PCR primer. When dsRNA is desired both types of PCR products may be included in the T7 transcription reaction. Alternatively, sense and anti-sense RNA may be mixed together after transcription, under annealing conditions, to form ds RNA.

Construction of Expression Plasmid Encoding a Fold-Back Type of RNA

An expression plasmid encoding an inverted repeat of a portion of the UP_{—}11 or OM_{—}10 gene may be constructed using the information disclosed in this application. A DNA fragment encoding an UP_{—}11 or OM_{—}10 foldback transcript may be prepared by PCR amplification and introduced into suitable restriction sites of a vector which includes the elements required for transcription of the UP_{—}11 or OM_{—}10 foldback transcript. The DNA fragment would encode a transcript that contains a fragment of the UP_{—}11 or OM_{—}10 gene of approximately at least 600 nucleotides in length, followed by a spacer sequence of at least 10 bp but not more than 200 bp, followed by the reverse complement of the UP_{—}11 or OM_{—}10 sequence chosen. CHO cells transfected with the construct will produce only fold-back RNA in which complementary target gene sequences form a double helix.

Assay

Balb/c mice (5 mice/group) may be injected intercranially with the murine UP_{—}11 or OM_{—}10 chain specific RNAs described above or with controls at doses ranging between 10 μg and 500 μg. Brains are harvested from a sample of the mice every four days for a period of three weeks and assayed for UP_{—}11 or OM_{—}10 levels using the antibodies as disclosed herein or by northern blot analysis for reduced RNA levels.

According to the present invention, mice receiving ds RNA molecules derived from both the UP_{—}11 or OM_{—}10 mRNA, UP_{—}11 or OM_{—}10 hnRNA and ds RNA derived from the UP_{—}11 or OM_{—}10 promoter demonstrate a reduction or inhibition in UP_{—}11 or OM_{—}10 production. A modest, if any, inhibitory effect is observed in sera of mice receiving the single stranded UP_{—}11 or OM_{—}10 derived RNA molecules, unless the RNA molecules have the capability of forming some level of double-strandedness.

### Example 14

### Method of the Invention in the Prophylaxis of Disease

In vivo Assay

Using the UP_{—}11 or OM_{—}10R specific RNA molecules described in Example 10, which do not have the ability to make UP_{—}11 or OM_{—}10 protein and UP_{—}11 or OM_{—}10 specific RNA molecules as controls, mice may be evaluated for protection from UP_{—}11 or OM_{—}10 related disease through the use of the injected UP_{—}11 or OM_{—}10 specific RNA molecules of the invention.

Balb/c mice (5 mice/group) may be immunized by intercranial injection with the described RNA molecules at doses ranging between 10 and 500 μg RNA. At days 1, 2, 4 and 7 following RNA injection, the mice may be observed for signs of UP_{—}11 or OM_{—}10 related phenotypic change.

According to the present invention, because the mice that receive dsRNA molecules of the present invention which contain the UP_{—}11 or OM_{—}10 sequence may be shown to be protected against UP_{—}11 or OM_{—}10 related disease. The mice receiving the control RNA molecules may be not protected. Mice receiving the ss RNA molecules which contain the UP_{—}11 or OM_{—}10 sequence may be expected to be minimally, if at all, protected, unless these molecules have the ability to become at least partially double stranded in vivo.

According to this invention, because the dsRNA molecules of the invention do not have the ability to make UP_{—}11 or OM_{—}10 protein, the protection provided by delivery of the RNA molecules to the animal is due to a non-immune mediated mechanism that is gene specific.

### Example 15

### RNA Interference in Drosophila and Chinese Hamster Cultured Cells

To observe the effects of RNA interference, either cell lines naturally expressing UP_{—}11 or OM_{—}10 can be identified and used or cell lines which express UP_{—}11 or OM_{—}10 as a transgene can be constructed by well known methods (and as outlined herein). As examples, the use of Drosophila and CHO cells are described. Drosophila S2 cells and Chinese hamster CHO-K1 cells, respectively, may be cultured in Schneider medium (Gibco BRL) at 25° C. and in Dulbecco's modified Eagle's medium (Gibco BRL) at 37° C. Both media may be supplemented with 10% heat-inactivated fetal bovine serum (Mitsubishi Kasei) and antibiotics (10 units/ml of penicillin (Meiji) and 50 μg/ml of streptomycin (Meiji)).

Transfection and RNAi Activity Assay

S2 and CHO-K1 cells, respectively, are inoculated at 1×10⁶ and 3×10⁵ cells/ml in each well of 24-well plate. After 1 day, using the calcium phosphate precipitation method, cells are transfected with UP_{—}11 or OM_{—}10 dsRNA (80 pg to 3 μg). Cells may be harvested 20 h after transfection and UP_{—}11 or OM_{—}10 gene expression measured.

### Example 16

### Antisense Inhibition in Vertebrate Cell Lines

Antisense can be performed using standard techniques including the use of kits such as those of Sequitur Inc. (Natick, Mass.). The following procedure utilizes phosphorothioate oligodeoxynucleotides and cationic lipids. The oligomers are selected to be complementary to the 5′ end of the mRNA so that the translation start site is encompassed.

1) Prior to plating the cells, the walls of the plate are gelatin coated to promote adhesion by incubating 0.2% sterile filtered gelatin for 30 minutes and then washing once with PBS. Cells are grown to 40-80% confluence. Hela cells can be used as a positive control.

2) the cells are washed with serum free media (such as Opti-MEMA from Gibco-BRL).

3) Suitable cationic lipids (such as Oligofectibn A from Sequitur, Inc.) are mixed and added to serum free media without antibiotics in a polystyrene tube. The concentration of the lipids can be varied depending on their source. Add oligomers to the tubes containing serum free media/cationic lipids to a final concentration of approximately 200 nM (50-400 nM range) from a 100 μM stock (2 μl per ml) and mix by inverting.

4) The oligomer/media/cationic lipid solution is added to the cells (approximately 0.5 mL for each well of a 24 well plate) and incubated at 37° C. for 4 hours.

5) The cells are gently washed with media and complete growth media is added. The cells are grown for 24 hours. A certain percentage of the cells may lift off the plate or become lysed.

Cells are harvested and UP_{—}11 or OM_{—}10 gene expression is measured.

### Example 17

### Production of Transfected Cell Strains by Gene Targeting

Gene targeting occurs when transfecting DNA either integrates into or partially replaces chromosomal DNA sequences through a homologous recombinant event. While such events can occur in the course of any given transfection experiment, they are usually masked by a vast excess of events in which plasmid DNA integrates by nonhomologous, or illegitimate, recombination.

Generation of a Construct Useful for Selection of Gene Targeting Events in Human Cells

One approach to selecting the targeted events is by genetic selection for the loss of a gene function due to the integration of transfecting DNA. The human HPRT locus encodes the enzyme hypoxanthine-phosphoribosyl transferase. Hprt-cells can be selected for by growth in medium containing the nucleoside analog 6-thioguanine (6-TG): cells with the wild-type (HPRT+) allele are killed by 6-TG, while cells with mutant (hprt−) alleles can survive. Cells harboring targeted events which disrupt HPRT gene function are therefore selectable in 6-TG medium.

To construct a plasmid for targeting to the HPRT locus, the 6.9 kb HindIII fragment extending from positions 11,960-18,869 in the HPRT sequence (Genebank name HUMHPRTB; Edwards et al., 1990) and including exons 2 and 3 of the HPRT gene, may be subdcloned into the HindIII site of pUC12. The resulting clone is cleaved at the unique XhoI site in exon 3 of the HPRT gene fragment and the 1.1 kb SalI-XhoI fragment containing the neo gene from pMC1 Neo (Stratagene) is inserted, disrupting the coding sequence of exon 3. One orientation, with the direction of neo transcription opposite that of HPRT transcription was chosen and designated pE3Neo. The replacement of the normal HPRT exon 3 with the neo-disrupted version will result in an hprt-, 6-TG resistant phenotype. Such cells will also be G418 resistant.

Generation of a Construct for Targeted Insertion of a Gene of Therapeutic Interest into the Human Genome and its use in Gene Tarqeting

A variant of pE3Neo, in which an UP_{—}11 or OM_{—}10 gene is inserted within the HPRT coding region, adjacent to or near the neo gene, can be used to target the UP_{—}11 or OM_{—}10 gene to a specific position in a recipient primary or secondary cell genome. Such a variant of pE3Neo can be constructed for targeting the UP_{—}11 or OM_{—}10gene to the HPRT locus.

A DNA fragment containing the UP_{—}11 or OM 10 gene and_linked mouse metallothionein (mMT) promoter is constructed. Separately, pE3Neo is digested with an enzyme which cuts at the junction of the neo fragment and HPRT exon 3 (the 3′ junction of the insertion into exon 3). Linearized pE3Neo fragment may be ligated to the UP_{—}11 or OM_{—}10-mMT fragment.

Bacterial colonies derived transfection with the ligation mixture are screened by restriction enzyme analysis for a single copy insertion of the UP_{—}11 or OM_{—}10-mMT fragment. An insertional mutant in which the UP_{—}11 or OM_{—}10 DNA is transcribed in the same direction as the neo gene is chosen and designated pE3Neo/UP_{—}11 or OM_{—}10. pE3Neo/UP_{—}11 or OM_{—}10 is digested to release a fragment containing HPRT, neo and mMT-UP_{—}11 or OM_{—}10 sequences. Digested DNA is treated and transfected into primary or secondary human fibroblasts. G418^{r} TG^{r} colonies are selected and analyzed for targeted insertion of the mMT-UP_{—}11 or OM_{—}10 and neo sequences into the HPRT gene. Individual colonies may be assayed for UP_{—}11 or OM_{—}10 expression using antibodies as described elsewhere herein.

Secondary human fibroblasts may be transfected with pE3Neo/UP_{—}11 or OM_{—}10 and thioguanine-resistant colonies analyzed for stable UP_{—}11 or OM_{—}10 expression and by restriction enzyme and Southern hybridization analysis.

The use of homologous recombination to target an UP_{—}11 or OM_{—}10 gene to a specific position in a cell's genomic DNA can be expanded upon and made more useful for producing products for therapeutic purposes (e.g., pharmaceuticals, gene therapy) by the insertion of a gene through which cells containing amplified copies of the gene can be selected for by exposure of the cells to an appropriate drug selection regimen. For example, pE3neo/UP_{—}11 or OM_{—}10 can be modified by inserting the dhfr, ada, or CAD gene at a position immediately adjacent to the UP_{—}11 or OM_{—}10 or neo genes in pE3neo/UP_{—}11 or OM_{—}10. Primary, secondary, or immortalized cells are transfected with such a plasmid and correctly targeted events are identified. These cells are further treated with increasing concentrations of drugs appropriate for the selection of cells containing amplified genes (for dhfr, the selective agent is methotrexate, for CAD the selective agent is N-(phosphonacetyl)-L-aspartate (PALA), and for ada the selective agent is an adenine nucleoside (e.g., alanosine). In this manner the integration of the gene of therapeutic interest will be coamplified along with the gene for which amplified copies are selected. Thus, the genetic engineering of cells to produce genes for therapeutic uses can be readily controlled by preselecting the site at which the targeting construct integrates and at which the amplified copies reside in the amplified cells.

Construction of Targeting Plasmids for Placing the UP 11 or OM 10 Gene Under the Control of the Mouse Metallothionein Promoter in Primary, Secondarv and Immortalized Human Fibroblasts

The following serves to illustrate one embodiment of the present invention, in which the normal positive and negative regulatory sequences upstream of the UP_{—}11 or OM_{—}10 gene are altered to allow expression of UP_{—}11 or OM_{—}10 in primary, secondary or immortalized human fibroblasts or other cells which do not express UP_{—}11 or OM_{—}10 in significant quantities.

Unique sequences of SEQ ID NO:1, SEQ ID NO:2, SEQ ID NO:3, SEQ ID NO:5, SEQ ID NO:6, SEQ ID NO:8 or SEQ ID NO:10 are selected which are located upstream from the UP_{—}11 or OM_{—}10 coding region and ligated to the mouse metallothionein promoter as targeting sequences. Typically, the 1.8 kb EcoRI-BglII from the mMT-I gene (containing no mMT coding sequences; Hamer and Walling, 1982); this fragment can also be isolated by known methods from mouse genomic DNA using PCR primers designed from analysis of mXT sequences available from Genbank; i.e., MUSMTI, MUSMTIP, MUSMTIPRM) is made blunt-ended by known methods and ligated with the 5′ UP_{—}11 or OM_{—}10 sequences. The orientations of resulting clones are analyzed and suitable DNAs are used for targeting primary and secondary human fibroblasts or other cells which do not express UP_{—}11 or OM_{—}10 in significant quantities.

Additional upstream sequences are useful in cases where it is desirable to modify, delete and/or replace negative regulatory elements or enhancers that lie upstream of the initial target sequence.

The cloning strategies described above allow sequences upstream of UP_{—}11 or OM_{—}10 to be modified in vitro for subsequent targeted transfection of primary, secondary or immortalized human fibroblasts or other cells which do not express UP_{—}11 or OM_{—}10 in significant quantities. The strategies describe simple insertions of the mMT promoter, and allow for deletion of the negative regulatory region, and deletion of the negative regulatory region and replacement with an enhancer with broad host-cell activity.

Targeting to Sequences Flanking the UP 11 or OM 10 Gene and Isolation of Targeted Primary, Secondary and Immortalized Human Fibroblasts by Screening

Targeting fragment containing the mMT promoter and UP_{—}11 or OM_{—}10 upstream sequences may be purified by phenol extraction and ethanol precipitation and transfected into primary or secondary human fibroblasts. Transfected cells are plated onto 150 mm dishes in human fibroblast nutrient medium. 48 hours later the cells are plated into 24 well dishes at a density of 10,000 cells/cm² (approximately 20,000 cells per well) so that, if targeting occurs at a rate of 1 event per 106 clonable cells then about 50 wells would need to be assayed to isolate a single expressing colony. Cells in which the transfecting DNA has targeted to the homologous region upstream of UP_{—}11 or OM_{—}10 will express UP_{—}11 or OM_{—}10 under the control of the mMT promoter. After 10 days, whole well supernatants are assayed for UP_{—}11 or OM_{—}10 expression. Clones from wells displaying UP_{—}11 or OM_{—}10 synthesis are isolated using known methods, typically by assaying fractions of the heterogenous populations of cells separated into individual wells or plates, assaying fractions of these positive wells, and repeating as needed, ultimately isolating the targeted colony by screening 96-well microtiter plates seeded at one cell per well. DNA from entire plate lysates can also be analyzed by PCR for amplification of a fragment using primers specific for the targeting sequences. Positive plates are trypsinized and replated at successively lower dilutions, and the DNA preparation and PCR steps repeated as needed to isolate targeted cells.

Targeting to Sequences Flanking the Human UP_{—}11 or OM_{—}10 Gene and Isolation of Targeted Primary, Secondary and Immortalized Human Fibroblasts by a Positive or a Combined Positive/Negative Selection System

Construction of 5′ UP_{—}11 or OM_{—}10-mMT targeting sequences and derivatives of such with additional upstream sequences can include the additional step of inserting the neo gene adjacent to the mMT promoter. In addition, a negative selection marker, for example, gpt (from PMSG (Pharmacia) or another suitable source), can be inserted. In the former case, G418^{r} colonies are isolated and screened by PCR amplification or restriction enzyme and Southern hybridization analysis of DNA prepared from pools of colonies to identify targeted colonies. In the latter case, G418^{r} colonies are placed in medium containing 6-thioxanthine to select against the integration of the gpt gene (Besnard et al., 1987). In addition, the HSV-TK gene can be placed on the opposite side of the insert to gpt, allowing selection for neo and against both gpt and TK by growing cells in human fibroblast nutrient medium containing 400 μg/ml G418, 100 μM 6-thioxanthine, and 25 μg/ml gancyclovir. The double negative selection should provide a nearly absolute selection for true targeted events and Southern blot analysis provides an ultimate confirmation.

The targeting schemes herein described can also be used to activate UP_{—}11 or OM_{—}10 expression in immortalized human cells (for example, HT1080 fibroblasts, HeLa cells, MCF-7 breast cancer cells, K-562 leukemia cells, KB carcinoma cells or 2780AD ovarian carcinoma cells) for the purposes of producing UP_{—}11 or OM_{—}10 for conventional pharmaceutical delivery.

The targeting constructs described and used in this example can be modified to include an amplifiable selectable marker (e.g., ada, dhfr, or CAD) which is useful for selecting cells in which the activated endogenous gene, and the amplifiable selectable marker, are amplified. Such cells, expressing or capable of expressing the endogenous gene encoding an UP_{—}11 or OM_{—}10 product can be used to produce proteins for conventional pharmaceutical delivery or for gene therapy.

### REFERENCES

European Application No. EP 0036776

European Application No. EP 0859055

European Application No. EP 125023

European Application No. EP 171496

European Application No. EP 173494

European Application No. EP 184187

European Application No. EP 264166

International Application No. WO 00/06597

International Application No. WO 00/40724

International Application No. WO 00/63364

International Application No. WO 00/49162

International Application No. WO 01/09184

International Application No. WO 86/01533

International Application No. WO 90/02809

International Application No. WO 90/11354

International Application No. WO 91/01140

International Application No. WO 91/17271

International Application No. WO 92/01047

International Application No. WO 92/09690

International Application No. WO 92/15679

International Application No. WO 92/18619

International Application No. WO 92/20791

International Application No. WO 93/01288

International Application No. WO 93/04169

International Application No. WO 94/10300

International Application No. WO 94/12650

International Application No. WO 94/16101

International Application No. WO 96/05302

International Application No. WO 97/07668

International Application No. WO 97/07669

International Application No. WO 98/20040

International Application No. WO 99/15650

Japanese Application No. JP08245697-A

U.S. Pat. No. 4,522,811

U.S. Pat. No. 4,554,101

U.S. Pat. No. 4,683,195

U.S. Pat. No. 4,683,202

U.S. Pat. No. 4,736,866

U.S. Pat. No. 4,870,009

U.S. Pat. No. 4,873,191

U.S. Pat. No. 4,873,316

U.S. Pat. No. 4,987,071

U.S. Pat. No. 5,116,742

U.S. Pat. No. 5,223,409

U.S. Pat. No. 5,283,317

U.S. Pat. No. 5,328,470

U.S. Pat. No. 5,498,531

U.S. Pat. No. 5,968,502

U.S. Pat. No. 5,968,502

U.S. Pat. No. 6,054,297

U.S. Statutory Invention Registration No. H1,892

Altschul et al., “Gapped BLAST and PSI-BLAST: a new generation of protein database search programs” Nucleic Acids Res. 25:3389-3402, 1997.

Altschul et al, J. Molec. Biol. 215:403-410, 1990.

Ausubel et al., Current Protocols in Molecular Biology, eds. Ausubel et al., John Wiley & Sons, 1992.

Baldari et al., Embo J 6:229-234, 1987.

Banerji et al., Cell, 33:729-740; 1983.

Bartel and Szostak, Science 261:1411-1418, 1993.

Bartel et al. Biotechniques 14:920-924, 1993(b).

Bartel, “Cellular Interactions and Development: A Practical Approach”, pp.153-179, 1993(a).

Besnard et al, Mol. Cell. Biol. 7:4139-4141, 1987.

Bradley, Current Opinion in Biotechnology 2:823-829, 1991.

Bradley, in “Teratocarcinomas and Embryonic Stem Cells: A Practical Approach,” E. J. Robertson, ed., IRL, Oxford, pp.113-152, 1987.

Bunzow et al., Nature, 336:783-787, 1988.

Burge and Karlin, “Prediction of complete gene structures in human genomic DNA.”J. Mol. Biol. 268:78-94, 1997.

Byrne and Ruddle, PNAS 86:5473-5477, 1989.

Calame and Eaton, Adv. Immunol 43:235-275, 1988.

Campes and Tilghman, Genes Dev. 3:537-546, 1989.

Chen et al, PNAS 91:3054-3057, 1994.

Cohen et al., Adv. Chromatogr. 36:127-162, 1996.

Cotton, Mutat. Res. 285:125-144, 1993.

Doestschman et al., J. Embryol. Exp. Morphol. 87:27-45, 1985.

Edlund et al., Science 230:912-916, 1985.

Edwards, A. et al., Genomics 6:593-608, 1990.

Eichelbaum, Clin. Exp. Pharmacol. Physiol, 23(10-11):983-985, 1996.

Elledge et al., Proc. Natl. Acad. Sci. USA, 88:1731-1735, 1991.

Feng and Doolittle, J. Mol. Evol. 35:351-360, 1987.

Finely and Brent, Proc. Natl. Acad. Sci. USA, 91:12980-12984, 1994.

Frohman et al., Proc. Natl. Acad. Sci. USA 85, 8998-9002, 1988.

Gaultier et al., Nucleic Acids Res. 15:6625-6641, 1987.

Griffin et al., Appl. Biochem. Biotechnol. 38:147-159, 1993.

Harner and Walling, J. Mol. Appl. Gen. 1:273 288, 1982.

Harlow and Lane, “Antibodies: A Laboratory Manual,” Cold Spring Harbor Laboratory Press, Cold Spring Harbor, N.Y., 1988

Harper et al., Cell, 75:805-816, 1993.

Harrington et al., Nature Biotechnology, 19:440-445, 2001.

Haselhoff and Gerlach, Nature 334:585-591, 1988.

Hayashi, Genet. Anal. Tech. Appl. 9:73-79, 1992.

Helene et al., Ann. N. YAcad Sci. 660:27-36, 1992.

Helene, Anticancer Drug Des. 6(6):569-84, 1991.

Heng et al., PNAS 89: 9509-9513, 1992.

Heng and Tsui, Chromosoma 102:325-332, 1993.

Henikoff and Henikoff, Proc. Natl. Acad. Sci. USA 89:10915, 1989.

Higgins and Sharp, CABIOS5:151-153, 1989.

Hogan, “Manipulating the Mouse Embryo,” Cold Spring Harbor Laboratory Press, Cold Spring Harbor, N.Y., 1986.

Inoue et al., FEBS Lett. 215:327-330, 1987(a).

Inoue et al., Nucleic Acids Res. 15:6131-6148, 1987(b).

Iwabuchi et al., Oncogene 8:1693-1696, 1993

Johnson et al., Endoc. Rev., 10:317-331, 1989.

Karlin and Altschul, Proc. Natl. Acad. Sci. USA 90:5873-5787, 1993.

Kaufman et al., EMBO J. 6:187-195, 1987.

Kessel and Gruss, Science 249:3 74-379, 1990.

Kobilka et al., Proc. Natl. Acad. Sci., USA, 84:46-50, 1987(a).

Kobilka et al., Science, 238:650-656, 1987(b).

Kurjan and Herskowitz, Cell 933-943, 1982.

Kyte and Doolittle, J. Mol. Biol., 157:105-132, 1982.

Lakso et al., PJVAS 89:6232-6236, 1992.

Lee et al., “Cloning and characterization of additional members of the G protein-coupled receptor family” Biochimica et Biophysica Acta. 1490(3):311-23, 2000.

Lefkowitz, Nature, 351:353-354, 1991.

Li et al., Cell 69:915, 1992.

Liu et al., “A serotonin-4 receptor-like pseudogene in humans” Brain Research.

Molecular Brain Research. 53(1-2):98-103, 1998.

Lucklow and Summers, Virology 170:31-39, 1989.

Madura et al., J. Biol. Chem. 268:12046-1205, 1993

Maher, Bioassays 14(12):807-15, 1992.

Mansour et al., Nature 336:348, 1988

Maxim and Gilbert, PNAS 74:560, 1977.

Morin et al., Nucleic Acids Res., 21:2157-2163, 1993.

Myers et al., Nature 313:495, 1985(a).

Myers et al., Science 230:1242, 1985(b).

Needleman and Wunsch, J. Mol. Biol. 48:443, 1970.

O'Gonnan et al., Science 251:1351-1355, 1991.

Orita et al., PNAS 86:2766, 1989.

Pearson and Lipman, Proc. Natal. Acad. Sci. USA 85:2444, 1988.

Saleeba et al., Meth. Enzymol. 217:286-295, 1992.

Sambrook et al., “Molecular Cloning: A Laboratory Manual” 2nd ed, Cold Spring Harbor Laboratory, Cold Spring Harbor Laboratory Press, Cold Spring Harbor, N.Y., 1989

Sanger, PNAS 74:5463, 1977.

Schultz et al., Gene 54:113-123, 1987

Simon, et al., Science, 252:802-8, 1991.

Smith and Waterman, Adv. Appl. Math. 2:482, 1981.

Smith et al., Mol. Cell Biol. 3:2156-2165, 1983.

Stadel et al., “Orphan G protein-coupled receptors: a neglected opportunity for pioneer drug discovery” TiPS 18:430-437, 1997.

Studier et al. “Gene Expression Technology” Methods in Enzymology 185, 60-89, 1990.

Thomas and Capecchi, Cell 51:503, 1987.

Wilmut et al., Nature 385:810-813, 1997.

Wilson et al., Cell 37:767, 1984.

Winoto and Baltimore. EMBO J. 8:729-733, 1989.

Zervos et al., Cell 72:223-232, 1993.

What is claimed is:

1. An isolated polynucleotide comprising a nucleic acid sequence which encodes a polypeptide comprising the amino acid sequence of SEQ ID NO:4.

2. The polynucleotide of claim 1, further comprising nucleic acid sequences encoding a heterologous protein.

3. A recombinant expression vector comprising the polynucleotide of claim 1.

4. The vector of claim 3, wherein the polynucleotide comprises the nucleic acid sequence of SEQ ID NO:1, SEQ ID NO:2 or SEQ ID NO:3.

5. A genetically engineered host cell, transfected, transformed or infected with the vector of claim 3.

6. The host cell of claim 5, wherein the host cell is a mammalian host cell.

7. An isolated polynucleotide comprising a nucleic acid sequence which encodes a polypeptide comprising the amino acid sequence of SEQ ID NO:7.

8. The polynucleotide of claim 7, further comprising nucleic acid sequences encoding a heterologous protein.

9. A recombinant expression vector comprising the polynucleotide of claim 7.

10. The vector of claim 9, wherein the polynucleotide comprises the nucleic acid sequence of SEQ ID NO:5 or SEQ ID NO:6.

11. A genetically engineered host cell, transfected, transformed or infected with the vector of claim 9.

12. The host cell of claim 11, wherein the host cell is a mammalian host cell.

13. An isolated polynucleotide comprising a nucleic acid sequence which encodes a polypeptide comprising the amino acid sequence of SEQ ID NO:9.

14. The polynucleotide of claim 13, further comprising nucleic acid sequences encoding a heterologous protein.

15. A recombinant expression vector comprising the polynucleotide of claim 13.

16. The vector of claim 15, wherein the polynucleotide comprises the nucleic acid sequence of SEQ ID NO:8.

17. A genetically engineered host cell, transfected, transformed or infected with the vector of claim 15.

18. The host cell of claim 17, wherein the host cell is a mammalian host cell.

19. An isolated polynucleotide comprising a nucleic acid sequence which encodes a polypeptide comprising the amino acid sequence of SEQ ID NO:11.

20. The polynucleotide of claim 19, further comprising nucleic acid sequences encoding a heterologous protein.

21. A recombinant expression vector comprising the polynucleotide of claim 19.

22. The vector of claim 21, wherein the polynucleotide comprises the nucleic acid sequence of SEQ ID NO:10.

23. A genetically engineered host cell, transfected, transformed or infected with the vector of claim 21.

24. The host cell of claim 23, wherein the host cell is a mammalian host cell.

25. An isolated polypeptide comprising the amino acid sequence of SEQ ID NO:4.

26. An isolated polypeptide comprising the amino acid sequence of SEQ ID NO:7.

27. An isolated polypeptide comprising the amino acid sequence of SEQ ID NO:9.

28. An isolated polypeptide comprising the amino acid sequence of SEQ ID NO:11.

29. An isolated polynucleotide comprising the nucleic acid sequence of SEQ ID NO:1 or a degenerate variant thereof.

30. The polynucleotide of claim 41, wherein the coding region of SEQ ID NO:1 comprises nucleotides 298 through 1,653.

31. An RNA molecule which is antisense to a polynucleotide comprising the nucleic acid sequence of SEQ ID NO:1 or a degenerate variant thereof.

32. The RNA of claim 31, wherein the RNA is antisense to the polynucleotide of SEQ ID NO:1 from about nucleotide 1 to about nucleotide 297 or from about nucleotide 1,654 to about nucleotide 3,824.

33. An isolated polynucleotide comprising the nucleic acid sequence of SEQ ID NO:2 or a degenerate variant thereof.

34. The polynucleotide of claim 33, wherein the coding region of SEQ ID NO:2 comprises nucleotides 1 through 1,313.

35. An RNA molecule which is antisense to a polynucleotide comprising the nucleic acid sequence of SEQ ID NO:2 or a degenerate variant thereof.

36. The RNA of claim 35, wherein the RNA is antisense to the polynucleotide of SEQ ID NO:2 from about nucleotide 1,314 to about nucleotide 3,405.

37. An isolated polynucleotide comprising the nucleic acid sequence of SEQ ID NO:3 or a degenerate variant thereof.

38. The polynucleotide of claim 37, wherein the coding region of SEQ ID NO:3 comprises nucleotides 671 through 2,026.

39. An RNA molecule which is antisense to a polynucleotide comprising the nucleic acid sequence of SEQ ID NO:3 or a degenerate variant thereof.

40. The RNA of claim 39, wherein the RNA is antisense to the polynucleotide of SEQ ID NO:3 from about nucleotide 1 to about nucleotide 670 or from about nucleotide 2,027 to about nucleotide 3,779.

41. An isolated polynucleotide comprising the nucleic acid sequence of SEQ ID NO:5 or a degenerate variant thereof.

42. The polynucleotide of claim 41, wherein the coding region of SEQ ID NO:5 comprises nucleotides 684 through 2,033.

43. An RNA molecule which is antisense to a polynucleotide comprising the nucleic acid sequence of SEQ ID NO:5 or a degenerate variant thereof.

44. The RNA of claim 43, wherein the RNA is antisense to the polynucleotide of SEQ ID NO:5 from about nucleotide 1 to about nucleotide 683 or from about nucleotide 2,034 to about nucleotide 3,384.

45. An isolated polynucleotide comprising the nucleic acid sequence of SEQ ID NO:6 or a degenerate variant thereof.

46. The polynucleotide of claim 45, wherein the coding region of SEQ ID NO:6 comprises nucleotides 685 through 2,034.

47. An RNA molecule which is antisense to a polynucleotide comprising the nucleic acid sequence of SEQ ID NO:6 or a degenerate variant thereof.

48. The RNA of claim 47, wherein the RNA is antisense to the polynucleotide of SEQ ID NO:6 from about nucleotide 1 to about nucleotide 684 or from about nucleotide 2,034 to about nucleotide 3,384.

49. An isolated polynucleotide comprising the nucleic acid sequence of SEQ ID NO:8 or a degenerate variant thereof.

50. The polynucleotide of claim 49, wherein the coding region of SEQ ID NO:8 comprises nucleotides 332 through 1,858.

51. An RNA molecule which is antisense to a polynucleotide comprising the nucleic acid sequence of SEQ ID NO:8 or a degenerate variant thereof.

52. The RNA of claim 51, wherein the RNA is antisense to the polynucleotide of SEQ ID NO:8 from about nucleotide 1 to about nucleotide 331 or from about nucleotide 1,859 to about nucleotide 4,718.

53. An isolated polynucleotide comprising the nucleic acid sequence of SEQ ID NO:10 or a degenerate variant thereof.

54. The polynucleotide of claim 53, wherein the coding region of SEQ ID NO:10 comprises nucleotides 250 through 1,785.

55. An RNA molecule which is antisense to a polynucleotide comprising the nucleic acid sequence of SEQ ID NO:10 or a degenerate variant thereof.

56. The RNA of claim 55, wherein the RNA is antisense to the polynucleotide of SEQ ID NO:10 from about nucleotide 1 to about nucleotide 249 or from about nucleotide 1,786 to about nucleotide 5,386.

57. A polynucleotide comprising a nucleic acid sequence which would hybridize to SEQ ID NO:1, or the complement of SEQ ID NO:1, under stringent conditions.

58. A polynucleotide comprising a nucleic acid sequence which would hybridize to SEQ ID NO:2, or the complement of SEQ ID NO:2, under stringent conditions.

59. A polynucleotide comprising a nucleic acid sequence which would hybridize to SEQ ID NO:3, or the complement of SEQ ID NO:3, under stringent conditions.

60. A polynucleotide comprising a nucleic acid sequence which would hybridize to SEQ ID NO:5, or the complement of SEQ ID NO:5, under stringent conditions.

61. A polynucleotide comprising a nucleic acid sequence which would hybridize to SEQ ID NO:6, or the complement of SEQ ID NO:6, under stringent conditions.

62. A polynucleotide comprising a nucleic acid sequence which would hybridize to SEQ ID NO:8, or the complement of SEQ ID NO:8, under stringent conditions.

63. A polynucleotide comprising a nucleic acid sequence which would hybridize to SEQ ID NO:10, or the complement of SEQ ID NO:10, under stringent conditions.

64. An antibody which selectively binds to a protein according to claims 25, 26, 27 or 28.

65. An antibody which selectively binds to an OM_{—}10 polypeptide, wherein the antibody binds an amino acid sequence comprising SEQ ID NO:12, SEQ ID NO:13, SEQ ID NO:14, SEQ ID NO:15 or SEQ ID NO:16.

66. An antibody which selectively binds an OM_{—}10 polypeptide fragment selected from the group consisting of SEQ ID NO:12, SEQ ID NO:13, SEQ ID NO:14, SEQ ID NO:15 and SEQ ID NO:16.

67. A human OM_{—}10 polypeptide comprising one or more epitopes selected from the group consisting of SEQ ID NO:12, SEQ ID NO:13, SEQ ID NO:14, SEQ ID NO:15 and SEQ ID NO:16.

68. An antibody which selectively binds to an UP_{—}11 polypeptide, wherein the antibody binds an amino acid sequence comprising SEQ ID NO:17, SEQ ID NO:18, SEQ ID NO:19, SEQ ID NO:20 or SEQ ID NO:21.

69. An antibody which selectively binds to an UP_{—}11 polypeptide fragment selected from the group consisting of SEQ ID NO:17, SEQ ID NO:18, SEQ ID NO:19, SEQ ID NO:20 and SEQ ID NO:21.

70. A human UP_{—}11 polypeptide comprising one or more epitopes selected from the group consisting of SEQ ID NO:17, SEQ ID NO:18, SEQ ID NO:19, SEQ ID NO:20 and SEQ ID NO:21.

71. A transgenic animal comprising a polynucleotide encoding a GPCR polypeptide comprising the amino acid sequence selected from the group consisting of SEQ ID NO:4, SEQ ID NO:7, SEQ ID NO:9 and SEQ ID NO:11.

72. A method for inhibiting the expression of a GPCR polynucleotide in a cell, the polynucleotide selected from the group consisting of SEQ ID NO:1, SEQ ID NO:2, SEQ ID NO:3, SEQ ID NO:5, SEQ ID NO:6, SEQ ID NO:8 and SEQ ID NO:10, the method comprising provided the cell with a nucleic acid molecule antisense to the polynucleotide.

73. A method for assaying the effects of test compounds on the activity of a GPCR polypeptide comprising the steps of:
(a) providing a transgenic animal comprising a polynucleotide encoding a GPCR polypeptide having an amino acid sequence selected from the group consisting of SEQ ID NO:4, SEQ ID NO:7, SEQ ID NO:9 and SEQ ID NO:11;
(b) administering a test compound to the animal; and
(c) determining the effects of the test compound on the activity of the GPCR in the presence and absence of the test compound.

74. A method for assaying the effects of test compounds on the activity of a GPCR polypeptide comprising the steps of:
(a) providing recombinant cells comprising a GPCR polypeptide having an amino acid sequence selected from the group consisting of SEQ ID NO:4, SEQ ID NO:7, SEQ ID NO:9 and SEQ ID NO11;
(b) contacting the cells with a test compound; and
(c) determining the effects of the test compound on the activity of the GPCR in the presence and absence of the test compound.

75. A method for the treatment of a subject in need of enhanced GPCR activity comprising:
(a) administering to the subject a therapeutically effective amount of an agonist to the GPCR receptor; and/or
(b) administering to the subject a polynucleotide encoding a GPCR polypeptide comprising an amino acid sequence selected from the group consisting of SEQ ID NO:4, SEQ ID NO:7, SEQ ID NO:9 and SEQ ID NO:11, in a form so as to effect the production of the GPCR activity in vivo.

76. A method for the treatment of a subject in need of inhibiting GPCR activity comprising:
(a) administering to the subject a therapeutically effective amount of an antagonist to the GPCR receptor; and/or
(b) administering to the subject a polynucleotide that inhibits the expression of a polynucleotide encoding a GPCR polypeptide comprising an amino acid sequence selected from the group consisting of SEQ ID NO:4, SEQ ID NO:7, SEQ ID NO:9 and SEQ ID NO:11; and/or
(c) administering to the subject a therapeutically effective amount of a polypeptide that competes with a GPCR for its ligand.

77. A method for the diagnosis of a disease or the susceptibility to a disease in a subject related to the expression or activity of a GPCR in the subject comprising:
(a) determining the presence or absence of a mutation in a polynucleotide encoding a GPCR polypeptide comprising an amino acid sequence selected from the group consisting of SEQ ID NO:4, SEQ ID NO:7, SEQ ID NO:9, and SEQ ID NO:11; and/or
(b) assaying for the presence of GPCR expression in a sampled derived from the subject, wherein the GPCR expressed is a polynucleotide encoding a GPCR polypeptide comprising an amino acid sequence selected from the group consisting of SEQ ID NO:4, SEQ ID NO:7, SEQ ID NO:9, and SEQ ID NO:11.

78. A method for the treatment of a subject having in need of the inhibition of GPCR activity, such treatment comprising administering to the patient a therapeutically effective amount of an antibody which binds to an extracellular portion of a GPCR polypeptide comprising an amino acid sequence selected from the group consisting of SEQ ID NO:4, SEQ ID NO:7, SEQ ID NO:9, and SEQ ID NO:11.

79. An isolated mammalian gene comprising a nucleic acid sequence of SEQ ID NO:1.

80. The gene of claim 79, wherein the gene encodes an UP_{—}11 protein comprising an amino acid of SEQ ID NO:4.

81. An isolated mammalian gene comprising a nucleic acid sequence of SEQ ID NO:2.

82. The gene of claim 81, wherein the gene encodes an UP_{—}11 protein comprising an amino acid of SEQ ID NO:4.

83. An isolated mammalian gene comprising a nucleic acid sequence of SEQ ID NO:3.

84. The gene of claim 83, wherein the gene encodes an UP_{—}11 protein comprising an amino acid of SEQ ID NO:4.

85. An isolated mammalian gene comprising a nucleic acid sequence of SEQ ID NO:5.

86. The gene of claim 85, wherein the gene encodes an UP_{—}11 protein comprising an amino acid of SEQ ID NO:7.

87. An isolated mammalian gene comprising a nucleic acid sequence of SEQ ID NO:6.

88. The gene of claim 87, wherein the gene encodes an UP_{—}11 protein comprising an amino acid of SEQ ID NO:7.

89. An isolated mammalian gene comprising a nucleic acid sequence of SEQ ID NO:8.

90. The gene of claim 89, wherein the gene encodes an OM_{—}10 protein comprising an amino acid of SEQ ID NO:9.

91. An isolated mammalian gene comprising a nucleic acid sequence of SEQ ID NO:10.

92. The gene of claim 91, wherein the gene encodes an OM_{—}10 protein comprising an amino acid of SEQ ID NO:11.

93. A method of activating expression and amplifying an endogenous OM_{—}10 gene in genomic DNA of a mammalian cell, wherein the OM_{—}10 gene is not expressed at significant levels in the cell as obtained, comprising the steps of:
(a) transfecting cells with polynucleotide sequences comprising:
(1) exogenous polynucleotide regulatory sequences not normally functionally linked to the endogenous OM_{—}10 gene in the cell as obtained;
(2) polynucleotide sequences homologous with OM_{—}10 gene sequences at a preselected site in the cells; and
(3) amplifiable polynucleotide sequences encoding a selectable marker,
thereby producing cells comprising the polynucleotide sequences;
(b) maintaining the cells produced in step (a) under conditions appropriate for homologous recombination to occur between polynucleotide sequences of step (a)(2) and OM_{—}10 gene sequences, thereby producing homologously recombinant mammalian cells having the polynucleotide sequences of steps (a)(1), (a)(2) and (a)(3) integrated into the OM_{—}10 gene and exogenous polynucleotide sequences of step (a)(1) functionally linked to the endogenous gene; and
(c) culturing the cells of step (b) under conditions which select for amplification of the amplifiable polynucleotide sequence encoding a selectable marker, whereby the amplifiable polynucleotide sequence and the endogenous OM_{—}10 gene functionally linked polynucleotide sequences of step (a)(1) are coamplified,
thereby producing homologously recombinant cells containing amplified polynucleotide sequences encoding a selectable marker and coamplified endogenous OM_{—}10 gene functionally linked to the polynucleotide sequence of step (a)(1), in which the coamplified OM_{—}10 gene is expressed.

94. A homologously recombinant cell produced by the method of claim 83.

95. A method of activating expression and amplifying an endogenous UP_{—}11 gene in genomic DNA of a mammalian cell, wherein the UP11 gene is not expressed at significant levels in the cell as obtained, comprising the steps of:
(a) transfecting cells with polynucleotide sequences comprising:
(1) exogenous polynucleotide regulatory sequences not normally functionally linked to the endogenous UP_{—}11 gene in the cell as obtained;
(2) polynucleotide sequences homologous with UP_{—}11 gene sequences at a preselected site in the cells; and
(3) amplifiable polynucleotide sequences encoding a selectable marker,
thereby producing cells comprising the polynucleotide sequences;
(b) maintaining the cells produced in step (a) under conditions appropriate for homologous recombination to occur between polynucleotide sequences of step (a)(2) and UP_{—}11 gene sequences, thereby producing homologously recombinant mammalian cells having the polynucleotide sequences of steps (a)(1), (a)(2) and (a)(3) integrated into the UP_{—}11 gene and exogenous polynucleotide sequences of step (a)(1) functionally linked to the endogenous gene; and
(c) culturing the cells of step (b) under conditions which select for amplification of the amplifiable polynucleotide sequence encoding a selectable marker, whereby the amplifiable polynucleotide sequence and the endogenous UP_{—}11 gene functionally linked polynucleotide sequences of step (a)(1) are coamplified,
thereby producing homologously recombinant cells containing amplified polynucleotide sequences encoding a selectable marker and coamplified endogenous UP_{—}11 gene functionally linked to the polynucleotide sequence of step (a)(1), in which the coamplified UP_{—}11 gene is expressed.

96. A homologously recombinant cell produced by the method of claim 95.

97. A method for providing an OM_{—}10 protein to a mammal comprising introducing into the mammal a homologously recombinant cell which produces the OM_{—}10 protein, the homologously recombinant cell being generated by the method comprising:
(a) providing a mammalian cell, the genomic DNA of which comprises an endogenous OM_{—}10 gene;
(b) providing a DNA construct comprising a targeting sequence of the OM_{—}10 gene, which is homologous to a target site upstream of the endogenous OM_{—}10 gene, an exogenous regulatory sequence, an exon and an unpaired splice-donor site at the 3′ end of the exon, wherein the exogenous regulatory sequence is operatively linked to the exon and;
(c) transfecting the cell of step (a) with the DNA construct of step (b),
thereby generating a homologously recombinant cell in which the splice-donor site is operatively linked to the second exon of the endogenous gene and the exogenous regulatory sequence controls transcription of the construct-derived exon, the endogenous OM_{—}10 gene and any sequence between the construct-derived exon and the endogenous OM_{—}10 gene, to produce an RNA transcript that encodes an OM_{—}10 protein.

98. A method for providing an UP_{—}11 protein to a mammal comprising introducing into the mammal a homologously recombinant cell which produces the UP_{—}11 protein, the homologously recombinant cell being generated by the method comprising:
(a) providing a mammalian cell, the genomic DNA of which comprises an endogenous UP_{—}11 gene;
(b) providing a DNA construct comprising a targeting sequence of the UP_{—}11 gene, which is homologous to a target site upstream of the endogenous UP_{—}11 gene, an exogenous regulatory sequence, an exon and an unpaired splice-donor site at the 3′ end of the exon, wherein the exogenous regulatory sequence is operatively linked to the exon and;
(c) transfecting the cell of step (a) with the DNA construct of step (b),
thereby generating a homologously recombinant cell in which the splice-donor site is operatively linked to the second exon of the endogenous gene and the exogenous regulatory sequence controls transcription of the construct-derived exon, the endogenous UP_{—}11 gene and any sequence between the construct-derived exon and the endogenous UP_{—}11 gene, to produce an RNA transcript that encodes an UP 11 protein.


SEQUENCE LISTING (25 sequences)

SEQ ID NO: 1 - DNA, 3824 bp, Homo sapiens
  exon (298)..(1653)
gtcgacccacgcgtccgagtgggtcaggctcctgcacctctcacgtctcctgcttcttagcagtcaccaaggcagaccctgcagctacctccggccagaaaggggatgagcttctgatccttcagctgcctggcctggcgctctgtacgcagacaaacctgcccaagaggctccagtgggaggtgccccctacgaaaccaggaagcctgggcctgggctcgccatcccagggtcgctggactaggatgggggatgggcctgtgacaggaggtaccctgggtgccctctttcggccccatggagtcctcacccatcccccagtcatcagggaactcttccactttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcgggggccccccagaccaccccacaccggacgtttgggggagggaaagcagcagtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcatgatgggccgctggacactcggagggatatggggctggggccagttatgattgcaaggaccaccttgtgggatcaccttttcccagctggctagggctgaggctggggtctctgcacacagcttttgcttagtgtttcctgggtcaggaacagagccaacaggatgaacgtgtgcaaaagccttggacttggctgtgatctttgactgctaggggagggaacctgggtatggtgagacggtgacgagagaaaagggtcacaaaggactggcctccctgatctctctcctcatggcagcgacccacctccagtcccctggacaatcgggtacaagagacttaaggttgggcatgggaagggtggggtttccatgatccattaaatgccttcctactcccattcatcgctctcaaaattagcttcagtgacaaagacttaaatctctctcctatctgcagcactgggttggagagagggcacgggagttggtcttggctgttcattgattgagactgtaggaactgtgttggttggtattggtggtggtattttcaacaaacagggaataactgcaaactggacaggacacccatctgggaccacctgtccatcctacttccctcaattgaatcaggtaacactaacggatcaaggcagggccagagggtggtgtggtctctatttgaacaaattcctggctcactgagcatcaaaaggggaaatgggctggtgggagtgggatagtctcccatttaagcagctaataaataatttttatgataaaaggttatactgataacaacattgactcctttagttcaattcagtgcataatagttgaacacccactagtccctgggacccacacagggcgtgtggtcattgcttttaaggagttcatagtctaagttgatgagataccttatattttcacaaagcactttgatttgataaagcactacagaatgtgcttgagaaatatattggagaatatgtccatggctctaacttctgagagttcagcccgtggcagcaagatgcataccttgaagcttcctgcagattgtggaaagcataggggttgtaaatgaaactctctaatgaagaaaaaaaattaaatgaaactgggcaaacagctttccccctttgttctaggaaaatttctaggttgtcttcctaccactagattattataccagtctagtgcctattacattgtggaagttccctattaaaataaatgcatacagaggaatcaatcattcctagacagggaaaaaactcttctttcaaacaccactgatcagctattagatccaaggaattgccagcaggtggcagtgtgagcccaatggaaggaggaaaggcgagtgtacgtggtgggaggaggaaggggagggcattaaacattgcctggcagccattttgttaatttattttgccttttcctttgactttgccctccagcccttccttcacatacatcaaagaagaaagttttaagagcaagggtatctttaattcaggctgaaatttcctgacactgtgatctcactggtgtttattacagagtttgacatacatgggttcatttgccatttatttttccctgtaggagtggatcatgaaggaaataaaaatttctcttttattatgctgagaactttcccaacaatttctgctatgaccaccttccaggagttttctagtcaccagatgccttggtaaagttcaatacgtaatctttggctctgaaagctgttcctggacaaaatctgagctaactcactgaagaatcaacagattgaggcaaccatccggtcagttactttttcctgcatcctgctggtgttggggtaactcccaatcctagatgaaaaccttagactttctgttgtcaggtgtccccaggcaatatcctacgggggcatgatagaaaagggtaactctggggtcagatagatgtacttactcactgtgtgaagttgggaaagctgcttaatttctctgagcctacttcctcacctgtaaaaatggggatcattattacctacctcacagggttgttgtgaggattaagagatgggatgtgggagcacctagccgtatctggcaaataggtactcaataaatactggttttacttcccaaaaaaaaaaaaaaagggcggccgc

SEQ ID NO: 2 - DNA, 3554 bp, Homo sapiens
  exon (1)..(1317)
ctttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcgggggccccccagaccaccccacaccggacgtttgggggagggaaagcagcagtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcatgatgggccgctggacactcggagggatatggggctggggccagttatgattgcaaggaccaccttgtgggatcaccttttcccagctggctagggctgaggctggggtctctgcacacagcttttgcttagtgtttcctgggtcaggaacagagccaacaggatgaacgtgtgcaaaagccttggacttggctgtgatctttgactgctaggggagggaacctgggtatggtgagacggtgacgagagaaaagggtcacaaaggactggcctccctgatctctctcctcatggcagcgacccacctccagtcccctggacaatcgggtacaagagacttaaggttgggcatgggaagggtggggtttccatgatccattaaatgccttcctactcccattcatcgctctcaaaattagcttcagtgacaaagacttaaatctctctcctatctgcagcactgggttggagagagggcacgggagttggtcttggctgttcattgattgagactgtaggaactgtgttggttggtattggtggtggtattttcaacaaacagggaataactgcaaactggacaggacacccatctgggaccacctgtccatcctacttccctcaattgaatcaggtaacactaacggatcaaggcagggccagagggtggtgtggtctctatttgaacaaattcctggctcactgagcatcaaaaggggaaatgggctggtgggagtgggatagtctcccatttaagcagctaataaataatttttatgataaaaggttatactgataacaacattgactcctttagttcaattcagtgcataatagttgaacacccactagtccctgggacccacacagggcgtgtggtcattgcttttaaggagttcatagtctaagttgatgagataccttatattttcacaaagcactttgatttgataaagcactacagaatgtgcttgagaaatatattggagaatatgtccatggctctaacttctgagagttcagcccgtggcagcaagatgcataccttgaagcttcctgcagattgtggaaagcataggggttgtaaatgaaactctctaatgaagaaaaaaaattaaatgaaactgggcaaacagctttccccctttgttctaggaaaatttctaggttgtcttcctaccactagattattataccagtctagtgcctattacattgtggaagttccctaaaaacatagtatatatagggaggagagtcctttgtgattgaaaaacatgttcacctctcctccctattaaaataaatgcatacagaggaatcaatcattcctagacagggaaaaaactcttctttcaaacaccactgatcagctattagatccaaggaattgccagcaggtggcagtgtgagcccaatggaaggaggaaaggcgagtgtacgtggtgggaggaggaaggggagggcattaaacattgcctggcagccattttgttaatttattttgccttttcctttgactttgccctccagcccttccttcacatacatcaaagaagaaagttttaagagcaagggtatctttaattcaggctgaaatttcctgacactgtgatctcactggtgtttattacagagtttgacatacatgggttcatttgccatttatttttccctgtaggagtggatcatgaaggaaataaaaatttctcctttattatgctgagaactttcccaacaatttctgctatgaccaccttccaggagttttctagtcaccagatgccttggtaaagttcaatacgtaatctttggctctgaaagctgttcctggacaaaatctgagctaactcactgaagaatcaacagattgaggcaaccatccggtcagttactttttcctgcatcctgctggtgttggggtaactcccaatcctagatgaaaaccttagactttctgttgtcaggtgtccccaggcaatatcctacgggggcatgatagaaaagggtaactctggggtcagatagatgtacttactcactgtgtgaagttgggaaagctgcttaatttctctgagcctacttcctcacctgtaaaaatggggatcattattacctacctcacagggttgttgtgaggattaagagatgggatgtgggagcacctagccgtatctggcaaataggtactcaataaatactggttttacttccaaaaaaaaaaaaaaaaaaaaaagcggccgc

SEQ ID NO: 3 - DNA, 3779 bp, Homo sapiens
  exon (671)..(2026)
gtcgacccacgcgtccgaagcatcagctgagaggagctatcacatgggagccgggactgctcagcaaagatggatttatgaggaaactgaaattcagaagattcacagagttagtaatgcccagaactgggactagaaactaaattttgtgctccttctactccccagcagctcttgccattctgaggagacaagaaatcaggaaatttacataaggaaccctaaaactgaggcactatcccagagatcagcaggaccctgggaaggagaaacaggatttagaatccccggctaacagttctggaaagggtagaagggtatggagaacaagaatggcagaaaggagatggaaaaggaagaggtgaaggccattccgaaagcggagtgttgagtgggtcaggctcctgcacctctcacgtctcctgcttcttagcagtcaccaaggcagaccctgcagctacctccggccagaaaggggatgagcttctgatccttcagctgcctggcctggcgctctgtacgcagacaaacctgcccaagaggctccagtgggaggtgccccctacgaaaccaggaagcctgggcctgggctcgccatcccagggtcgctggactaggatgggggatgggcctgtgacaggaggtaccctgggtgccctctttcggccccatggagtcctcacccatcccccagtcatcagggaactcttccactttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcaggggccccccagaccaccccacaccggacgtttgggggagggaaagcagcagtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcatgatgggccgctggacactcggagggatatggggctggggccagttatgattgcaaggaccaccttgtgggatcaccttttcccagctggctagggctgaggctggggtctctgcacacagcttttgcttagtgtttcctgggtcaggaacagagccaacaggatgaacgtgtgcaaaagccttggacttggctgtgatctttgactgctaggggagggaacctgggtatggtgagacggtgacgagagaaaagggtcacaaaggactggcctccctgatctctctcctcatggcagcgacccacctccagtcccctggacaatcgggtacaagagacttaaggttgggcatgggaagggtggggtttccatgatccattaaatgccttcctactcccattcatcgctctcaaaattagcttcagtgacaaagacttaaatctctctcctatctgcagcactgggttggagagagggcacgggagttggtcttggctgttcattgattgagactgtaggaactgtgttggttggtattggtggtggtattttcaacaaacagggaataactgcaaactggacaggacacccatctgggaccacctgtccatcctacttccctcaattgaatcaggtaacactaacggatcaaggcagggccagagggtggtgtggtctctatttgaacaaattcctggctcactgagcatcaaaaggggaaatgggctggtgggagtgggatagtctcccatttaagcagctaataaataatttttatgataaaaggttatactgataacaacattgactcctttagttcaattcagtgcataatagttgaacacccactagtccctgggacccacacagggcgtgtggtcattgcttttaaggagttcatagtctaagttgatgagataccttatattttcacaaagcactttgatttgataaagcactacagaatgtgcttgagaaatatattggagaatatgtccatggctctaacttctgagagttcagcccgtggcagcaagatgcataccttgaagcttcctgcagattgtggaaagcataggggttgtaaatgaaactctctaatgaagaaaaaaaaaattaaatgaaactgggcaaacagctttccccctttgttctaggaaaatttctaggttgtcttcctaccactagattattataccagtctagtgcctattacattgtggaagttccctaaaaacatagtatatatagggaggagagtcctttgtgattgaaaaacatgttcacctctcctccctattaaaataaatgcatacagaggaatcaatcattcctagacagggaaaaaactcttctttcaaacaccactgatcagctattagatccaaggaattgccagcaggtggcagtgtgagcccaatggaaggaggaaaggcgagtgtacgtggtgggaggaggaaggggagggcattaaacattgcctggcagccattttgttaatttattttgccttttcctttgactttgccctccagcccttccttcacatacatcaaagaagaaagttttaagagcaagggtatctttaattcaggctgaaatttcctgacactgtgatctcactggtgtttattacagagtttgacatacatgggttcatttgccatttatttttccctgtaggagtggatcatgaaggaaataaaaatttctcttttattaaaaaaaaaaaaaaaaaaaaaaaaagggcggccgc

SEQ ID NO: 4 - protein, 451 aa, Homo sapiens
MESSPIPQSSGNSSTLGRVPQTPGPSTASGVPEVGLRDVASESVALFFMLLLDLTAVAGNAAVMAVIAKTPALRKFVFVFHLCLVDLLAALTLMPLAMLSSSALFDHALFGEVACRLYLFLSVCFVSLAILSVSAINVERYYYVVHPMRYEVRMTLGLVASVLVGVWVKALAMASVPVLGRVSWEEGAPSVPPGCSLQWSHSAYCQLFVVVFAVLYFLLPLLLILVVYCSMFRVARVAAMQHGPLPTWMETPRQRSESLSSRSTMVTSSGAPQTTPHRTFGGGKAAVVLLAVGGQFLLCWLPYFSFHLYVALSAQPISTGQVESVVTWIGYFCFTSNPFFYGCLNRQIRGELSKQFVCFFKPAPEEELRLPSREGSIEENFLQFLQGTGCPSESWVSRPLPSPKQEPPAVDFRIPGQIAEETSEFLEQQLTSDIIMSDSYLRPAASPRLES

SEQ ID NO: 5 - DNA, 3384 bp, Mus musculus
  exon (684)..(2033)
gtcgacccacgcgtccgcccacgcgtccggaggcatcagctgagaagagctatcacataggcgctgggagctgctcagcaaagatgccttcatgaggaaactggagtccggaagagttgcagagtgagtaatacccagacctggaactagaagctgaatctcatgctccttctacttcccattctgatgagaaaatcagaaatttcacaaaatcaaccctaaagccagagcactgtcctagagcaaagcaggaccctggagaggggagacaggaggatttagaattgccctcagaagggaagaagaacaaggagaactaggaaagaacgaacatggagaactaaaaaagaaagtgagaaaagaggtgccagaggtcactcggaaggccactgcagagtatgtgaggatcctacacagtgcttcccatcaccgggactgaccccggggctaccttctgacagaaactggacatgacctactgagtttggagcagcctggcctggcactctgtctacataggaacccagcttggaaggctagtgattagagcctgccttacaggctccagaaggccccccaacaaaattgggaagcctggacctgggcttacatcccagggttgtggagtaggatgggggatgggcctgtaacaggaagtgccctgggtgtcctctttcggccccatggagtcctcacccatcccccagtcatcaggaaactcgtccactttgggaagggcccttcaaaccccaggtccctctactgccagcggggtcccagagttgggattacgggacgtggcttcagaatctgtggccctcttcttcatgctcctgttggatctcactgctgtggctggcaatgctgctgtgatggctgttattgccaagacacccgccctccgaaaatttgtttttgtcttccatctttgtctggtggacctgctggctgccctgaccctcatgccgcttgccatgctctccagctctgccctctttgaccacgccctctttggggaggtggcctgccgcctctacctgttcctgagcgtttgctttgtcagcctggccatcctttcggtgtctgccattaatgtggagcgctactattatgtggtccacccaatgcgctacgaggtgcgcatgacactagggctggtggcctccgtgctggtgggcgtgtgggtaaaggccctagccatggcttctgtgccagtgttgggaagggtctactgggaggaaggagctcccagtgttaaccccggctgttctctccaatggagccatagtgcctactgccagctttttgtggtggtctttgctgttctgtacttcttgctgcccttgatcctgatctttgtggtctactgcagcatgtttcgagtggctcgcgtggctgccatgcaacacgggccgctgcccacgtggatggagacgccccggcaacgctctgagtctctcagtagccgctctactatggttaccagctccggggctcaccagaccaccccacaccggacgtttgggggtgggaaggcagcagtggtcctcctggctgtagggggacagttcttgctttgttggttgccctacttctctttccatctctatgttgccctgagcgcacagcccatttcagcaggacaggtggagaacgtggtaacctggattggctacttttgcttcacttccaacccctttttctacggatgtctcaaccgtcagatccggggcgagcttagcaaacagtttgtctgcttcttcaaggcagctccagaggaggagctgaggctgcctagtcgtgagggctccattgaggagaatttcctgcagttcctccaggggacctctgagaactgggtttctcggcccctacccagtcctaagcgggagccaccccctgttgttgactttcgaatcccaggccagattgctgaggagacctcagagttcctggagcagcaactcaccagcgacatcatcatgtccgacagctacctccgtcccgccccctcaccaaggctggagtcatgacggacaccaggagggaaataaagcttgggactggtttatgatttcaaggactgcttttgcggctggctggggtctgggctagggtctctggacttagcttttgcttggtgtttcctgggtcaggcccagaatcgacaggatggacatgtggcaaaaagccttggacttggctgggatctttgactattgggggagggaacctgggtatggtgagacgttgatgagagaaaagggtgacaaaggtgagggaaagcctttcttccagtgtactcttcaggcctcgggagacagggaaacttcctaagggtaggcggtggagcagcaggctaggaacagttaatctggggactgttgaggttgacctctttccagagtagtagtccagactaatgcttactctgagacaaggtaagaaagcggcccacatcttctcatttgccatctcggcaagtgtttcatgagttaacagatcccttcctaaagttaatgtctagagtgagaagacctgtaggggtgaattggatttggccagcagcaaggaaaaattgcaatcagggtagtggaggagaagacagaacaactctgcaactctctcctattctctttcctgcacatatgaaatcaagtgtgggtcctgacctcagcagagatgagcaggaggcaggatgccctttccctccttgtcttttgagtaactaggaatgactcgggggtcagagagctgagggtgggtgttagcctttgaattggtaacgtggctggatacagaaaggccaggtaaattactctgatcaataatactgccaatcttttctttccaggactggcctccccgatctatctcctcatggcagtgacccacttccagccccctggacattcgggtacaagagactcaggtcgggcaagggaagggtggggtttccattgtccatgaaatgtctccctgttccccatcattgctctcaaaattagcttctgtgacaaagacttaaatctgtctcctacctgcagccctaggttggagagagagcagagaattgggccttgctgcttattgattaagactataggggctgtgttggttagtatcagcaatggtattttcagcggaaagggattaattgcaagctggacaggtttcccctctgggtcctgttcattccatttccctcaactgaatactaaagggtcaaggtagaaccagagggggatgtgggctatagctgaacaaatttctggctcactcaacatgaaagggggaaagtgggttggggtggagtagtttcccctttgaacaaccaataaattttatgataaaaagttaaaaaaaaaaaaaaagggcggccgc

SEQ ID NO: 6 - DNA, 3397 bp, Mus musculus
  exon (685)..(2034)
gtcgacccacgcgtccgcagcagccttggagaggcatcagctgagaagagctatcacataggcgctgggagctgctcagcaaagatgccttcatgaggaaactggagtccggaagagttgcagagtgagtaatacccagacctggaactagaagctgaatctcatgctccttctacttcccattctgatgagaaaatcagaaatttcacaaaatcaaccctaaagccagagcactgtcctagagcaaagcaggaccctggagaggggagacaggaggatttagaattgccctcagaagggaagaagaacaaggagaactaggaaagaacgaacatggagaactaaaaaagaaagtgagaaaagaggtgccagaggtcactcggaaggccactgcagagtatgtgaggatcctacacagtgcttcccatcaccgggactgaccccggggctaccttctgacagaaactggacatgacctactgagtttggagcagcctggcctggcactctgtctacataggaacccagcttggaaggctagtgattagagcctgccttacaggctccagaaggccccccaacaaaattgggaagcctggacctgggcttacatcccagggttgtggagtaggatgggggatgggcctgtaacaggaagtgccctgggtgtcctctttcggccccatggagtcctcacccatcccccagtcatcaggaaactcgtccactttgggaagggcccttcaaaccccaggtccctctactgccagcggggtcccagagttgggattacgggacgtggcttcagaatctgtggccctcttcttcatgctcctgttggatctcactgctgtggctggcaatgctgctgtgatggctgttattgccaagacacccgccctccgaaaatttgtttttgtcttccatctttgtctggtggacctgctggctgccctgaccctcatgccgcttgccatgctctccagctctgccctctttgaccacgccctctttggggaggtggcctgccgcctctacctgttcctgagcgtttgctttgtcagcctggccatcctttcggtgtctgccattaatgtggagcgctactattatgtggtccacccaatgcgctacgaggtgcgcatgacactagggctggtggcctccgtgctggtgggcgtgtgggtaaaggccctagccatggcttctgtgccagtgttgggaagggtctactgggaggaaggagctcccagtgttaaccccggctgttctctccaatggagccatagtgcctactgccagctttttgtggtggtctttgctgttctgtacttcttgctgcccttgatcctgatctttgtggtctactgcagcatgtttcgagtggctcgcgtggctgccatgcaacacgggccgctgcccacgtggatggagacgccccggcaacgctctgagtctctcagtagccgctctactatggttaccagctccggggctcaccagaccaccccacaccggacgtttgggggtgggaaggcagcagtggtcctcctggctgtagggggacagttcttgctttgttggttgccctacttctctttccatctctatgttgccctgagcgcacagcccatttcagcaggacaggtggagaacgtggtaacctggattggctacttttgcttcacttccaacccctttttctacggatgtctcaaccgtcagatccggggcgagcttagcaaacagtttgtctgcttcttcaaggcagctccagaggaggagctgaggctgcctagtcgtgagggctccattgaggagaatttcctgcagttcctccaggggacctctgagaactgggtttctcggcccctacccagtcctaagcgggagccaccccctgttgttgactttcgaatcccaggccagattgctgaggagacctcagagttcctggagcagcaactcaccagcgacatcatcatgtccgacagctacctccgtcccgccccctcaccaaggctggagtcatgacggacaccaggagggaaataaagcttgggactggtttatgatttcaaggactgcttttgcggctggctggggtctgggctagggtctctggacttagcttttgcttggtgtttcctgggtcaggcccagaatcgacaggatggacatgtggcaaaaagccttggacttggctgggatctttgactattgggggagggaacctgggtatggtgagacgttgatgagagaaaagggtgacaaaggtgagggaaagcctttcttccagtgtactcttcaggcctcgggagacagggaaacttcctaagggtaggcggtggagcagcaggctaggaacagttaatctggggactgttgaggttgacctctttccagagtagtagtccagactaatgcttactctgagacaaggtaagaaagcggcccacatcttctcatttgccatctcggcaagtgtttcatgagttaacagatcccttcctaaagttaatgtctagagtgagaagacctgtaggggtgaattggatttggccagcagcaaggaaaaattgcaatcagggtagtggaggagaagacagaacaactctgcaactctctcctattctctttcctgcacatatgaaatcaagtgtgggtcctgacctcagcagagatgagcaggaggcaggatgccctttccctccttgtcttttgagtaactaggaatgactcgggggtcagagagctgagggtgggtgttagcctttgaattggtaacgtggctggatacagaaaggccaggtaaattactctgatcaataatactgccaatcttttctttccaggactggcctccccgatctatctcctcatggcagtgacccacttccagccccctggacattcgggtacaagagactcaggtcgggcaagggaagggtggggtttccattgtccatgaaatgtctccctgttccccatcattgctctcaaaattagcttctgtgacaaagacttaaatctgtctcctacctgcagccctaggttggagagagagcagagaattgggccttgctgcttattgattaagactataggggctgtgttggttagtatcagcaatggtattttcagcggaaagggattaattgcaagctggacaggtttcccctctgggtcctgttcattccatttccctcaactgaatactaaagggtcaaggtagaaccagagggggatgtgggctatagctgaacaaatttctggctcactcaacatgaaagggggaaagtgggttggggtggagtagtttcccctttgaacaaccaataaattttatgataaaaagttatattaatatcaaaaaaaaaaaaaaaagggcggccgc

SEQ ID NO: 7 - protein, 449 aa, Mus musculus
MESSPIPQSSGNSSTLGRALQTPGPSTASGVPELGLRDVASESVALFFMLLLDLTAVAGNAAVMAVIAKTPALRKFVFVFHLCLVDLLAALTLMPLAMLSSSALFDHALFGEVACRLYLFLSVCFVSLAILSVSAINVERYYYVVHPMRYEVRMTLGLVASVLVGVWVKALAMASVPVLGRVYWEEGAPSVNPGCSLQWSHSAYCQLFVVVFAVLYFLLPLILIFVVYCSMFRVARVAAMQHGPLPTWMETPRQRSESLSSRSTMVTSSGAHQTTPHRTFGGGKAAVVLLAVGGQFLLCWLPYFSFHLYVALSAQPISAGQVENVVTWIGYFCFTSNPFFYGCLNRQIRGELSKQFVCFFKAAPEEELRLPSREGSIEENFLQFLQGTSENWVSRPLPSPKREPPPVVDFRIPGQIAEETSEFLEQQLTSDIIMSDSYLRPAPSPRLES

SEQ ID NO: 8 - DNA, 4718 bp, Homo sapiens
  exon (332)..(1858)
gagaaagcgcacgcacgcacgcgccagcaggagaaacagatgagaggaaatcagagccctggagagagacaggcagacagatctggagagtccggaaaggagccatagaagctgcccgcactggggatggagccgtgcggaaacccggggtagggggtcctgcagcgtccttgctgggcgcggaggcttctccccttgacgggtgactaactctgcctgcgtgtttcttttgtcaccagcataggcactgagtgcggtctgtgcacccctttgccacccaccggtgccggcactgagcctgcaacctgtctcacgccctctggctgttgccatgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcttggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttgaagttagttctaaggcaaaccttgaactgtccataacacgagaaacaagaggagatttcttttcaatggacccacaattcattaatgccaaaccataccatttcaggcaaaggtgttgcacacacatgctcttcaccacaaggtagataaatatatagaagaggcaggaactggggtctttccgtaaaagcatggacttgaggattctgactgaaattttccccccaaagattattaggctctacatttcttaaagcaacaagggctatccattttggacttgtagttggtattctatcttttccagagctacaacatgccaactttagctctgaaggaaagggaagatgatgcttgtgaacttaaggacttttcggccctcgggtcgggagctcatgggccagagctacagcttgtgttcaactgaaagaaaggcaatggaccaaatcattcatggagcccaagaaacagaacctaatggactgatcaacatatgagccaaattctgaactgaacagccccacagtcgggtgcaaagactgttacacaaactaaaacaaagggcctcctacagttagaatctcaagaaggtttctagatcccctaaagggatccagaaaagtagaaggacatgtatgaaatgggaagctagtccaagggaaaagattgagaaataacacacatctggagagctaaacagttgactttttttcctataaaatcttgggtttatgcatgggctggaactgaggtcattaagtgtaaattgtcaattgacacaaatattttctgtctcctgtttgaataatagtggggcagaaatcatgccactattttacaacttcccttatgtgactgaattgagatgctggtgggaattcttcagatctctgccaacacttctgttttcttttggtttgtttttgtcaaataagcctttttttagtcaaacagtatttacagaaaaaaagaaattcaactagaagtggcctaagtcctacaaaattcatgatgtcactgaggaataatttgttcatcagaaatatattttgtgtccatgagatcatagacaataaatgtgatctccacatggggagcaaggaaggcagaatgaacatttttcttcctccaggcacacccatgtgtcttttccacctgtggctctctttaaagcttttaagctctctgcagatgtgaaagagaaatatcagagagtcagaaatgacaaagaggatgatttcacaatacctagaaaacatgtaacctattccaaacagtcctaaaatcagagcattcagatcagacatatcctaattaatgctgttgaaataaatcacgttgggaaaactttaacaatatctaaattatccctagggtcaattcacaggaacattcctcaaatcccaaaccgcaaaataactttgggcagggatatacatatacatttctgagggcatggaccgtatgtatgtgaccaagtaacatggaaccaaaaaaacagtcaagccagtgtttttgatcctcctacagaacaagttaaagcaactccagagtcaaccaactgttcatgcagaaatccactgtcaatatgggtggagggagtgtttggttgaaaatggttaatcaggtagttgtatgatgtaagatgaccatcttcagagtttagcctcattcttgtgtgattgtcatgcctttattagaactcaagtttcatttaaataaatgcccagctcatttattttttttatctcttcctcctcacagatttcaacatgaacttctcaaggggtaaacagcaatgtatttggactgtgaataactctgcatgggaatttgggattgccatgttcagaatttaggaaagtagagggaatagaaccaaataatatagagctgacccatccaataaaaataccatgataaccttattaattccaactccattaatttggaacttgtagttattcagacaaggcatgggggctaaagtttacccttacactatcatttatttttctaccaaaatgcataaagtgaattaacagtcataaatttgttctaccaatttattgccaaactcttgaactgacctttccttaaaggatatctgggtgaagaaatggcctatgtgatcaatcctcctacaggggaggggcagtgccttcaggtctgttcaaattgtcaaaggagttcaaggtagctatagcctatcctttgagtagaaatgcttacttgggtaggaaacaggatttcaaacataaatgtctccaaacattgtgttaaaactgaacttcttgttttatttttaaagctcacccccttcaaagtgtatcagagaaaattgtttgccaaaatctcaaatcaaaatggaaccagaacctgtacaagtatggtaagtccatttaacagcacacacaacattctcaagggcactgagattccctttcctttttgaagttcctcttttccctattttagtcattgtccattattttggtaaattggattcctcaaaagtgaagaccttttgaaactatgagcctggaaaagaggaccttttaattaagagcattctgctttgatgacattttcttctaaatgaacaataaggcctatggttagcttggagatagcaagtactcgaaattttttgctattttgaataaagcactatcaacttaatgaggttttactgcacatactgttttgtcatttgaaaatctgaaagcacacaaaaaaagtcatcattagcctcacagatcctcatgtgcaatagctttccctgaatgtttttaaaggatgtattcttttgccaaggccacttcatatgtgcagtaaaaaaaaaaaaaaaaaaaaaaaaa

SEQ ID NO: 9 - protein, 508 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVLVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAAKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 10 - DNA, 5386 bp, Mus musculus
  exon (250)..(1785)
gctggctggacgtacgggcatatactcggtgtcccgctcccgctgagcaccgctgctcctaccactcggtgcgagctctcagccgcctgtgccccgaaaggtggtcagaggaaacgcggcgagccccgagggatcggtctccggttcctgtggcgcgaagccttcagcagcaacagatcgtgtgcggtatcattgcccaccactccaaacacaaagctggaccttctgtcgtgcctgtctgacttcaccatgccacccagctgcactaacagtactcaagagaacaatggcagtcgagtgtgcctccccctctccaagatgcctattagtgtagctcacggcatcatccgctcagttgtgctgctcgtcatccttggtgtagcctttctgggtaacgtagtgctgggttatgtattgcaccgtaagccaaacttgctgcaggtgaccaaccggttcatatttaacctgcttgtcactgacctgctgcaggttgctctcgtggccccctgggtggtgtccactgccattcctttcttctggcctctcaacatccacttctgcactgccctggttagcctcacccacttatttgcctttgctagtgtcaataccattgtggtggtgtcagttgatcgttacctgaccatcatccaccctctttcctacccatccaagatgaccaaccgacgtagttatattctcctctatggcacctggattgcagccttcctgcagagcacacctccactctatggctggggccacgctacttttgatgaccgtaatgccttctgttccatgatctggggagccagccctgcctatacggttgtcagtgtggtatccttcctcgttattccactgggtgttatgattgcctgctattctgtggtgttcggtgcagcccggaggcagcaagctctcctgtataaggccaagagccaccgcttggaggtgagagtcgaggactctgtggtgcatgagaatgaagagggagcaaagaagagggatgagttccaggacaagaatgagttccagggccaagatggaggtggtcaggccgaggctaagggaagcagctccatggaagagagtcccatggtagccgagggcagcagccagaagaccggaaaaggaagcctggatttcagtgcaggtatcatggagggcaaggacagtgacgaggtcagtaatggcagcatggaggggctggaagtcatcactgaatttcaggctagcagcgcaaaggcagacaccggccgcatagatgccaatcagtgcaacattgacgtgggcgaagatgatgtagagtttggcatggatgaaattcatttcaacgacgatgttgaggcgatgcgcattccagagagcagtccacccagtcgtcgaaacagcaccagcgacccacctttgcctccatgctatgagtgcaaagctgctagagtgatcttcgtcatcatttccacttatgtgctatctctggggccctactgctttctagcagtgctggctgtgtgggtggatatcgataccagggtaccccagtgggtgatcaccataataatctggctttttttcctgcagtgttgcatccacccatatgtctatggctatatgcacaagagcatcaagaaggaaatccaggaggtactgaagaagttaatctgtaagaaaagcccccctgtagaagatagccaccctgaccttcatgaaacggaagctggtacagagggaggtattgaaggcaaggctgtcccctcccatgattcagctacttcaccttaaagttaacagtaaggcaaactttaattgtacacaaaaacagaacacaagagcagctttcttttcagcgctccgctcacaatctcattagtgccagtgcttaccatttcaggcaaaggggttgcgcgcacatcctttcccaccacacggcagataaataaaaggaagaagtagggacttggatctttcctgaaaagtataagcctgtcaaagcacggactttaggatccccaccaaatatatatacagatgtacacatattaggctctaaatttcccaaagcaaggactatctggtttggagctgttcttggtattctgcctgtctccccagaactatgacatgtcagctttagctctgaataaaaggaaaagcaatgcctacggacataaggactatttggccttcaggtcaggaactcatgggctagggctacatattgtgtgcagctgaaagaaaagaaattgaccaaatcaagcaaagctaggtggatggatcaccaaatgagcagatttctgtaccagagagtaccacagtatggtgcaaagactgtactgcaagctgcaacaaaggtggattacgcagatagactgtaaagaaagcttctaggtcttcaaaagggatccagaaaagggcaagcctgaaatgaggagctagggcaaaagaagaaatggagaaataaggcatatccacggagctaaacaactgtatgtttctttctctttctctctcttctctccttctccaccttcccctaccctgctacatgggcagggactgaccactgtgcaaatggaaaaaaggacaattgacacaaatgttttctgtctcctatttgaataacagcaggaaagaaatcaggccactattttactatttcccttctgcctctagacctctgaaagccactattttattttcttttatttctaactgattttttttattaaaaagtatttccaggtttcaagaagaaaggaagaaagaagaaagacagagagatagaaggaaaaaaaatcaagtatgaatggccaaagttctagaaaactcacactgccaataaattttatatcaagaaaatatcttttatctctgtaccgtaataaacatgaagtaggttttccatatgaggatagatatgtacatgcatagatatcttttcaacctgtggctctctctaaaacttgtaaattctctgtacaaatgcaggggagaatataaatgtcaggaatgatgttttattttttccttctcacagattccagcttgagcttctcaagggaggagaggtaaacggttgtgaattgtgagggtgtgggattaccatattcagaatgcaggagagttgatacaaccaaatagtattgaaatggcctgtcagattagtatattcacgagaagtttatcgacttccacttcactaatttaggacttgtgataacctcagacaaggcactgtatctaaagttaactctcacacttcccttcattttaacactctcactacttaactgtgtttctattttttttcttattactctaggcctctaaaggccccatgttactcccaaggttaggcatctgaggaggcagaaacgtgctggaaacaacaaactgtagcctaagacccatgaagaacaggcaccgtgccaattatatatggcttcttcctccatggcaaatgtttaatgtgcacactagcacagttagtctataagccacaaaacaggttagagaaagatacaggaaaagtaaatatactgaacttggctactgccaatcactggcaagttcattgctttttggtaaatagggtaagaatcctcaaagaacatgaaggctgccaatagaagttaggtttccatcattgcctccctaagcctccatatcttagcagtatacacactaaggggaaaccacagcaatgtgtacacttaagaaggtctgctgtgtgaagattaatatctgtctttctttgactctaaaagagacaaaaacaagattggttttagtttgctgtttcagacatgagtggatttcccccttttcattagttataactttattgaaaattgagtacttttgtcttgtgtcagtgatgtgttctcttgtggtatttcttctactgtaagttctaactgtatataaaattcgttcttggagataaggtgctagagattagattgtgtgtgtttgtggctagtgtcatcagtaaaatgagtgatgtgtgtgtgtacatgtaaagttagttaacaaaatgcatgcagtatcctatatgtgacccacaatttggtcactttttgaagtagaacatagtacattaatttacctttaaaagtgtatactacaggatatgtaacatggctccacagtggtattcgggaagaggtgcctttcattccttacatccctggtacgtgacaagcaagaacttattctggtacagctgggaatagatgtgaactaaattatcatcttggctgaagtcctcacctgcagttctccaccccactggcactggtatgcctgttttcttcaatacatagatagatctcaaaatcaaagaagacaagtcctttccccataaaagggtaagaaccccagggcaggctattggagtcctgatagcaggaggattttaaaagagtactgcagtttcaagacctaaacaggaaccagtacctgactggaaagttaatcagacttacatttagctgatccatagttggtgatccctgccctcctagagaagtgccaagacccagaagaaggctgctctgttttgtttttgttgttttcctctttggctgtctcagatgagatgcagcattaataaagaaacagtgagaattggggggtgggtggggggagggcagggattgaagctcaggtgtttgaagagttacagttgtagattaaatatatttgcagaagaactcagattattttattatattttgaaaacaaaagtattacaagaggatatatatttatatatattctcattaaatcatttctaaaactgcctttaataccaaatttcacgtgctattttgagactgagaacaataggacgagtgcactcagtcaataagacagttctctttaatactttccattttaaatctaaaactttccttttaaaacaaaagatttgtaatttaaaagtgccttttcaaaggattttcaaaactatagtcctggggccatgttcccattggcacaacttaccttcaggtgcacaaatgggtcggaatttattgtccacctgtcagcgagaagagaaatccctcacactcaaatcaaatttatgaattgatactgttacatgggcaggtcgtcccagagactctgcccaaagtcacggtttcatatattggttgattttaagtgaatgcattctaaactggttgtgatacctttagtgccagacaggaacaacagactcctgcttggggaatgaagagagattaacatttgtggtttaagtattattaatatttttcgtgtttcttaaataggtgctgtaaatctgttcttggtacattcttctgaaatatgctaaataaagtctcattttatgtgtgaaaaaaaaaaaaaaaaa

SEQ ID NO: 11 - protein, 511 aa, Mus musculus
MPPSCTNSTQENNGSRVCLPLSKMPISVAHGIIRSVVLLVILGVAFLGNVVLGYVLHRKPNLLQVTNRFIFNLLVTDLLQVALVAPWVVSTAIPFFWPLNIHFCTALVSLTHLFAFASVNTIVVVSVDRYLTIIHPLSYPSKMTNRRSYILLYGTWIAAFLQSTPPLYGWGHATFDDRNAFCSMIWGASPAYTVVSVVSFLVIPLGVMIACYSVVFGAARRQQALLYKAKSHRLEVRVEDSVVHENEEGAKKRDEFQDKNEFQGQDGGGQAEAKGSSSMEESPMVAEGSSQKTGKGSLDFSAGIMEGKDSDEVSNGSMEGLEVITEFQASSAKADTGRIDANQCNIDVGEDDVEFGMDEIHFNDDVEAMRIPESSPPSRRNSTSDPPLPPCYECKAARVIFVIISTYVLSLGPYCFLAVLAVWVDIDTRVPQWVITIIIWLFFLQCCIHPYVYGYMHKSIKKEIQEVLKKLICKKSPPVEDSHPDLHETEAGTEGGIEGKAVPSHDSATSP

SEQ ID NO: 12 - protein, 15 aa, Homo sapiens
CPLYGWGQAAFDERN

SEQ ID NO: 13 - protein, 15 aa, Homo sapiens
CVENEDEEGAEKKEE

SEQ ID NO: 14 - protein, 16 aa, Homo sapiens
CQHEGEVKAKEGRMEA

SEQ ID NO: 15 - protein, 15 aa, Homo sapiens
CSIDLGEDDMEFGED

SEQ ID NO: 16 - protein, 15 aa, Homo sapiens
CMLKKFFCKEKPPKE

SEQ ID NO: 17 - protein, 16 aa, Homo sapiens
CSSSALFDHALFGEVA

SEQ ID NO: 18 - protein, 15 aa, Homo sapiens
CGAPQTTPHRTFGGG

SEQ ID NO: 19 - protein, 15 aa, Homo sapiens
CFFKPAPEEELRLPS

SEQ ID NO: 20 - protein, 18 aa, Homo sapiens
CKQEPPAVDFRIPGQIAE

SEQ ID NO: 21 - protein, 15 aa, Homo sapiens
CLNRQIRGELSKQFV

SEQ ID NO: 22 - DNA, 42 bp, Homo sapiens
atgcatgcaagcttgcaccatgctcctgctggacttgactgc

SEQ ID NO: 23 - DNA, 32 bp, Homo sapiens
atgcatgcctcgagtgactccagccggggtga

SEQ ID NO: 24 - DNA, 42 bp, Homo sapiens
atgcatgcaagcttgcaccatgacgtccacctgcaccaacag

SEQ ID NO: 25 - DNA, 33 bp, Homo sapiens
atgcatgcctcgagaggaaaagtagcagaatcg